SEQUENCE LISTING (117 sequences)

SEQ ID NO: 1 - DNA, 49 bp, synthetic construct
  source 1..49: mol_type = other DNA
ggatgaattgtacaaaagatcttaaattgaattgaattgaaatcgatag

SEQ ID NO: 2 - DNA, 46 bp, synthetic construct
  source 1..46: mol_type = other DNA
ccctttgcaaatagtcctactagtaaataatatccttctcgaaagc

SEQ ID NO: 3 - DNA, 51 bp, synthetic construct
  source 1..51: mol_type = other DNA
aagggttgctcgagaaagagctcatacataacattttaagataagcaagtg

SEQ ID NO: 4 - DNA, 48 bp, synthetic construct
  source 1..48: mol_type = other DNA
tgaataattcttcacctttagacatcttttaatgttatcgatggattc

SEQ ID NO: 5 - DNA, 45 bp, synthetic construct
  source 1..45: mol_type = other DNA
aagggttgctcgagaaagagctctcttatcttgtatgcccgatat

SEQ ID NO: 6 - DNA, 51 bp, synthetic construct
  source 1..51: mol_type = other DNA
tgaataattcttcacctttagacattttatcttattgatcttctttgttta

SEQ ID NO: 7 - DNA, 43 bp, synthetic construct
  source 1..43: mol_type = other DNA
aagggttgctcgagaaagagctctgtcttgttgtgttgtgacg

SEQ ID NO: 8 - DNA, 44 bp, synthetic construct
  source 1..44: mol_type = other DNA
tgaataattcttcacctttagacatggctagaaaagaggaaggg

SEQ ID NO: 9 - DNA, 44 bp, synthetic construct
  source 1..44: mol_type = other DNA
aagggttgctcgagaaagagctcgaaattcaaaactctccagac

SEQ ID NO: 10 - DNA, 44 bp, synthetic construct
  source 1..44: mol_type = other DNA
tgaataattcttcacctttagacattggcacaaatgtggtttcc

SEQ ID NO: 11 - DNA, 41 bp, synthetic construct
  source 1..41: mol_type = other DNA
aagggttgctcgagaaagagctctgcgatactgccgtagcg

SEQ ID NO: 12 - DNA, 45 bp, synthetic construct
  source 1..45: mol_type = other DNA
tgaataattcttcacctttagacatgacccttttctcgatatgtt

SEQ ID NO: 13 - DNA, 42 bp, synthetic construct
  source 1..42: mol_type = other DNA
aagggttgctcgagaaagagctcccgccatctctactcactc

SEQ ID NO: 14 - DNA, 46 bp, synthetic construct
  source 1..46: mol_type = other DNA
tgaataattcttcacctttagacattgattttccagactcgtaaac

SEQ ID NO: 15 - DNA, 44 bp, synthetic construct
  source 1..44: mol_type = other DNA
aagggttgctcgagaaagagctcccggatatgaaaatggaatgc

SEQ ID NO: 16 - DNA, 46 bp, synthetic construct
  source 1..46: mol_type = other DNA
tgaataattcttcacctttagacatattctgcttagtttggccttc

SEQ ID NO: 17 - DNA, 46 bp, synthetic construct
  source 1..46: mol_type = other DNA
aagggttgctcgagaaagagctcagttcattgatgttgaagaagtg

SEQ ID NO: 18 - DNA, 47 bp, synthetic construct
  source 1..47: mol_type = other DNA
tgaataattcttcacctttagacattttttgtcctccttttcttgtg

SEQ ID NO: 19 - DNA, 47 bp, synthetic construct
  source 1..47: mol_type = other DNA
aacgacggccagtgaattcagtttaaacatgtactaatcagtctaac

SEQ ID NO: 20 - DNA, 60 bp, synthetic construct
  source 1..60: mol_type = other DNA
tggtatatgattttgtggacattttgcggccgctttatcttattgatcttctttgtttag

SEQ ID NO: 21 - DNA, 34 bp, synthetic construct
  source 1..34: mol_type = other DNA
gcggccgcaaaatgtccacaaaatcatataccag

SEQ ID NO: 22 - DNA, 35 bp, synthetic construct
  source 1..35: mol_type = other DNA
tctagatttgggcccgatcccaatacaacagatca

SEQ ID NO: 23 - DNA, 60 bp, synthetic construct
  source 1..60: mol_type = other DNA
ctgttgtattgggatcgggcccaaatctagatctaattggtttaattaataaatttaata

SEQ ID NO: 24 - DNA, 40 bp, synthetic construct
  source 1..40: mol_type = other DNA
cctcacgaagaagttaagcttgagcatcggaccgaagcat

SEQ ID NO: 25 - DNA, 40 bp, synthetic construct
  source 1..40: mol_type = other DNA
atgcttcggtccgatgctcaagcttaacttcttcgtgagg

SEQ ID NO: 26 - DNA, 50 bp, synthetic construct
  source 1..50: mol_type = other DNA
gtatgctatacgaagttattaggctcgagctcgagttaatttatctcatg

SEQ ID NO: 27 - DNA, 56 bp, synthetic construct
  source 1..56: mol_type = other DNA
ggaatctcggtcgtaatgatttgcatgcatacataacattttaagataagcaagtg

SEQ ID NO: 28 - DNA, 49 bp, synthetic construct
  source 1..49: mol_type = other DNA
gcagttcacataccagatggagccatcttttaatgttatcgatggattc

SEQ ID NO: 29 - DNA, 26 bp, synthetic construct
  source 1..26: mol_type = other DNA
atggctccatctggtatgtgaactgc

SEQ ID NO: 30 - DNA, 47 bp, synthetic construct
  source 1..47: mol_type = other DNA
gaccatgattacgccaagcttgtttaaactatgttccttgatacctc

SEQ ID NO: 31 - DNA, 46 bp, synthetic construct
  source 1..46: mol_type = other DNA
ggaatctcggtcgtaatgatttgcatgctgcgatactgccgtagcg

SEQ ID NO: 32 - DNA, 46 bp, synthetic construct
  source 1..46: mol_type = other DNA
gcagttcacataccagatggagccatgacccttttctcgatatgtt

SEQ ID NO: 33 - DNA, 49 bp, synthetic construct
  source 1..49: mol_type = other DNA
ggaatctcggtcgtaatgatttgcatgcgaaattcaaaactctccagac

SEQ ID NO: 34 - DNA, 45 bp, synthetic construct
  source 1..45: mol_type = other DNA
gcagttcacataccagatggagccattggcacaaatgtggtttcc

SEQ ID NO: 35 - DNA, 47 bp, synthetic construct
  source 1..47: mol_type = other DNA
ggaatctcggtcgtaatgatttgcatgcccgccatctctactcactc

SEQ ID NO: 36 - DNA, 47 bp, synthetic construct
  source 1..47: mol_type = other DNA
gcagttcacataccagatggagccattgattttccagactcgtaaac

SEQ ID NO: 37 - DNA, 48 bp, synthetic construct
  source 1..48: mol_type = other DNA
aacgacggccagtgaattcagtttaaactcttctgcttcgttcagctg

SEQ ID NO: 38 - DNA, 60 bp, synthetic construct
  source 1..60: mol_type = other DNA
gcacgtcaagactgtcaaggagggtattcgggcccgtatctttttttcttttttcaaacg

SEQ ID NO: 39 - DNA, 48 bp, synthetic construct
  source 1..48: mol_type = other DNA
gacttagattggtatatatacgcatatggaataccctccttgacagtc

SEQ ID NO: 40 - DNA, 50 bp, synthetic construct
  source 1..50: mol_type = other DNA
attgataatgataaactcgaactgactagtcgttagtatcgaatcgacag

SEQ ID NO: 41 - DNA, 60 bp, synthetic construct
  source 1..60: mol_type = other DNA
gtcgctatactgctgtcgattcgatactaacggcggccgcgagcaacggctttcttttgt

SEQ ID NO: 42 - DNA, 39 bp, synthetic construct
  source 1..39: mol_type = other DNA
acaaatgaaaagagatgcggccgtatggtgtgaaaatct

SEQ ID NO: 43 - DNA, 185 bp, synthetic construct
  source 1..185: mol_type = other DNA
agattttcacaccatacggccgcatctcttttcatttgtatttaaatccatttcaaattttatgtttagttcgagatcctcagttttcggcgcataggaaccacgtacataataactaaacataaatctataataaataaaaaacaacgatgggagctcgagcctaataacttcgtatagcatac

SEQ ID NO: 44 - DNA, 59 bp, synthetic construct
  source 1..59: mol_type = other DNA
gtatgctatacgaagttattaggctcgagctcccatcgttgttttttatttattataga

SEQ ID NO: 45 - DNA, 55 bp, synthetic construct
  source 1..55: mol_type = other DNA
ggaatctcggtcgtaatgatttgatatgaaggcatgctgcgatactgccgtagcg

SEQ ID NO: 46 - DNA, 51 bp, synthetic construct
  source 1..51: mol_type = other DNA
cgttgatgtcttcattagtctcgaagtccatgacccttttctcgatatgtt

SEQ ID NO: 47 - DNA, 31 bp, synthetic construct
  source 1..31: mol_type = other DNA
atggacttcgagactaatgaagacatcaacg

SEQ ID NO: 48 - DNA, 48 bp, synthetic construct
  source 1..48: mol_type = other DNA
gaccatgattacgccaagcttgtttaaacgtttccgtaagtgatcaac

SEQ ID NO: 49 - DNA, 60 bp, synthetic construct
  source 1..60: mol_type = other DNA
ggaatctcggtcgtaatgatttgatatgaaggcatgcagttcattgatgttgaagaagtg

SEQ ID NO: 50 - DNA, 53 bp, synthetic construct
  source 1..53: mol_type = other DNA
cgttgatgtcttcattagtctcgaagtccattttttgtcctccttttcttgtg

SEQ ID NO: 51 - DNA, 58 bp, synthetic construct
  source 1..58: mol_type = other DNA
ggaatctcggtcgtaatgatttgatatgaaggcatgcccggatatgaaaatggaatgc

SEQ ID NO: 52 - DNA, 52 bp, synthetic construct
  source 1..52: mol_type = other DNA
cgttgatgtcttcattagtctcgaagtccatattctgcttagtttggccttc

SEQ ID NO: 53 - DNA, 60 bp, synthetic construct
  source 1..60: mol_type = other DNA
gttgatgtcttcattagtctcgaagtctcctcctcccatattctgcttagtttggccttc

SEQ ID NO: 54 - DNA, 50 bp, synthetic construct
  source 1..50: mol_type = other DNA
aagggttgctcgagaaagagctcgtaaaaagaacaagaagagaataaaac

SEQ ID NO: 55 - DNA, 52 bp, synthetic construct
  source 1..52: mol_type = other DNA
tgaataattcttcacctttagacatttttcaatttatttgattgttggtttc

SEQ ID NO: 56 - DNA, 43 bp, synthetic construct
  source 1..43: mol_type = other DNA
aagggttgctcgagaaagagctcctcgaataataacggctctc

SEQ ID NO: 57 - DNA, 45 bp, synthetic construct
  source 1..45: mol_type = other DNA
tgaataattcttcacctttagacatgatcggtcgtgattatcttg

SEQ ID NO: 58 - DNA, 43 bp, synthetic construct
  source 1..43: mol_type = other DNA
aagggttgctcgagaaagagctccctcgtgttgttataacgac

SEQ ID NO: 59 - DNA, 49 bp, synthetic construct
  source 1..49: mol_type = other DNA
tgaataattcttcacctttagacattgttatacttgtggacttttattc

SEQ ID NO: 60 - DNA, 46 bp, synthetic construct
  source 1..46: mol_type = other DNA
aagggttgctcgagaaagagctcaacctgaagggacagagtttaac

SEQ ID NO: 61 - DNA, 55 bp, synthetic construct
  source 1..55: mol_type = other DNA
gtgaataattcttcacctttagacattgttaattcagtaaattttcgatcttggg

SEQ ID NO: 62 - DNA, 43 bp, synthetic construct
  source 1..43: mol_type = other DNA
aagggttgctcgagaaagagctccgtatccaattcggacgttg

SEQ ID NO: 63 - DNA, 45 bp, synthetic construct
  source 1..45: mol_type = other DNA
tgaataattcttcacctttagacattttcgtagatctcgggcttg

SEQ ID NO: 64 - DNA, 43 bp, synthetic construct
  source 1..43: mol_type = other DNA
aagggttgctcgagaaagagctcacgttgaagaattgagggag

SEQ ID NO: 65 - DNA, 47 bp, synthetic construct
  source 1..47: mol_type = other DNA
tgaataattcttcacctttagacattttgactgattgttgttgattg

SEQ ID NO: 66 - DNA, 43 bp, synthetic construct
  source 1..43: mol_type = other DNA
aagggttgctcgagaaagagctcaaacgccactaatcaacctg

SEQ ID NO: 67 - DNA, 50 bp, synthetic construct
  source 1..50: mol_type = other DNA
tgaataattcttcacctttagacatctaagaaaaacaatggggaaaatat

SEQ ID NO: 68 - DNA, 45 bp, synthetic construct
  source 1..45: mol_type = other DNA
aagggttgctcgagaaagagctcagctggaaaaatgcgtaataac

SEQ ID NO: 69 - DNA, 45 bp, synthetic construct
  source 1..45: mol_type = other DNA
tgaataattcttcacctttagacattgcgtctccttgtgcctgct

SEQ ID NO: 70 - DNA, 45 bp, synthetic construct
  source 1..45: mol_type = other DNA
aagggttgctcgagaaagagctccaacgtcaacataattcaatag

SEQ ID NO: 71 - DNA, 46 bp, synthetic construct
  source 1..46: mol_type = other DNA
tgaataattcttcacctttagacatatctcttgtttgactctccag

SEQ ID NO: 72 - DNA, 43 bp, synthetic construct
  source 1..43: mol_type = other DNA
aagggttgctcgagaaagagctcaccactgaggctagtgatct

SEQ ID NO: 73 - DNA, 49 bp, synthetic construct
  source 1..49: mol_type = other DNA
tgaataattcttcacctttagacattctcagaaattgagttatttatac

SEQ ID NO: 74 - DNA, 43 bp, synthetic construct
  source 1..43: mol_type = other DNA
aagggttgctcgagaaagagctcttgtggtgccactttccgta

SEQ ID NO: 75 - DNA, 46 bp, synthetic construct
  source 1..46: mol_type = other DNA
tgaataattcttcacctttagacattttttcacgcaagatgtgtac

SEQ ID NO: 76 - DNA, 44 bp, synthetic construct
  source 1..44: mol_type = other DNA
aagggttgctcgagaaagagctcgtttgatagcagtccattaac

SEQ ID NO: 77 - DNA, 46 bp, synthetic construct
  source 1..46: mol_type = other DNA
tgaataattcttcacctttagacattcgcgcttgctctaaactgtg

SEQ ID NO: 78 - DNA, 43 bp, synthetic construct
  source 1..43: mol_type = other DNA
aagggttgctcgagaaagagctcgtagcggatgacatcctgat

SEQ ID NO: 79 - DNA, 47 bp, synthetic construct
  source 1..47: mol_type = other DNA
tgaataattcttcacctttagacattcttgacagatcctttataatg

SEQ ID NO: 80 - DNA, 43 bp, synthetic construct
  source 1..43: mol_type = other DNA
aagggttgctcgagaaagagctcgcttgttctctcgttgatac

SEQ ID NO: 81 - DNA, 48 bp, synthetic construct
  source 1..48: mol_type = other DNA
tgaataattcttcacctttagacattgtcgtattttatacacacactg

SEQ ID NO: 82 - DNA, 43 bp, synthetic construct
  source 1..43: mol_type = other DNA
aagggttgctcgagaaagagctcctgcgggaacctcttccaca

SEQ ID NO: 83 - skipped

SEQ ID NO: 84 - DNA, 53 bp, synthetic construct
  source 1..53: mol_type = other DNA
acaggttccggttagcctgcgctagcttatattgaattttcaaaaattcttac

SEQ ID NO: 85 - DNA, 60 bp, synthetic construct
  source 1..60: mol_type = other DNA
tttattaattaaaccaattagatctaggggcccattgtagcaaagattgtaaggaaatag

SEQ ID NO: 86 - DNA, 49 bp, synthetic construct
  source 1..49: mol_type = other DNA
cattacttcatgagataaattaactcgagtgtactaatccaaggaggtt

SEQ ID NO: 87 - DNA, 50 bp, synthetic construct
  source 1..50: mol_type = other DNA
ctttgtctggagagttttgaatttcgagctcacgccacagaaacctcaga

SEQ ID NO: 88 - DNA, 57 bp, synthetic construct
  source 1..57: mol_type = other DNA
gtatcattacttcatgagataaattaactcgagtaaaccaattttatttgaacttgc

SEQ ID NO: 89 - DNA, 56 bp, synthetic construct
  source 1..56: mol_type = other DNA
cttaccttcttcaattttcattttggatccactgtaaaaaactttttttattatac

SEQ ID NO: 90 - DNA, 54 bp, synthetic construct
  source 1..54: mol_type = other DNA
gtataataaaaaaagttttttacagtggatccaaaatggaacacgttgctttcg

SEQ ID NO: 91 - DNA, 43 bp, synthetic construct
  source 1..43: mol_type = other DNA
ccaacttaccttcttcaatttttggacctgggttaagttcaac

SEQ ID NO: 92 - DNA, 52 bp, synthetic construct
  source 1..52: mol_type = other DNA
gctggtgacgttgaacttaacccaggtccaaaaattgaagaaggtaagttgg

SEQ ID NO: 93 - DNA, 60 bp, synthetic construct
  source 1..60: mol_type = other DNA
accaccaccaccaccaccgagctcaccagaacctggcttagtgattctagtttgggcatc

SEQ ID NO: 94 - DNA, 59 bp, synthetic construct
  source 1..59: mol_type = other DNA
ccaggttctggtgagctcggtggtggtggtggtggtgcttcagaaaaagaaattaggag

SEQ ID NO: 95 - DNA, 37 bp, synthetic construct
  source 1..37: mol_type = other DNA
catatcatcggcgaccaaccagtaagcctgcaacaac

SEQ ID NO: 96 - DNA, 37 bp, synthetic construct
  source 1..37: mol_type = other DNA
gttgttgcaggcttactggttggtcgccgatgatatg

SEQ ID NO: 97 - DNA, 47 bp, synthetic construct
  source 1..47: mol_type = other DNA
aaatcattacgaccgagattcccgggattgtagcaaagattgtaagg

SEQ ID NO: 98 - DNA, 54 bp, synthetic construct
  source 1..54: mol_type = other DNA
atcactaagccaggttctggttctggtagaagatcagctaactatcaaccatcc

SEQ ID NO: 99 - DNA, 45 bp, synthetic construct
  source 1..45: mol_type = other DNA
gaagcaccaccaccaccaccaccaccctttgtacctggtgatgcg

SEQ ID NO: 100 - DNA, 29 bp, synthetic construct
  source 1..29: mol_type = other DNA
ttagcttattctgaggtttctgtggcgtg

SEQ ID NO: 101 - DNA, 31 bp, synthetic construct
  source 1..31: mol_type = other DNA
tccggggtgttagactgattagtacatgttt

SEQ ID NO: 102 - DNA, 42 bp, synthetic construct
  source 1..42: mol_type = other DNA
aagggttgctcgagaaagagctccatatggcgtatccaagcc

SEQ ID NO: 103 - DNA, 59 bp, synthetic construct
  source 1..59: mol_type = other DNA
cacaaacacatactatcagaatacaggatccaaaatgtctaaaggtgaagaattattca

SEQ ID NO: 104 - DNA, 48 bp, synthetic construct
  source 1..48: mol_type = other DNA
cattacttcatgagataaattaactcgagcatatggcgtatccaagcc

SEQ ID NO: 105 - DNA, 48 bp, synthetic construct
  source 1..48: mol_type = other DNA
aaatcattacgaccgagattcccgggaaataatatccttctcgaaagc

SEQ ID NO: 106 - DNA, 49 bp, synthetic construct
  source 1..49: mol_type = other DNA
gctttcgagaaggatattatttcccgggccacagagaacaggagattac

SEQ ID NO: 107 - DNA, 59 bp, synthetic construct
  source 1..59: mol_type = other DNA
agatggcaaccacaaagacattttgtcgactgtaaatgtgtgtatatattatattatag

SEQ ID NO: 108 - DNA, 59 bp, synthetic construct
  source 1..59: mol_type = other DNA
ctataatataatatatacacacatttacagtcgacaaaatgtctttgtggttgccatct

SEQ ID NO: 109 - DNA, 60 bp, synthetic construct
  source 1..60: mol_type = other DNA
tccgccctgcaggtcactattaatgatggtgatggtggtgagcagttgtagaggtagaag

SEQ ID NO: 110 - DNA, 60 bp, synthetic construct
  source 1..60: mol_type = other DNA
actgctcaccaccatcaccatcattaatagtgacctgcagggcggattgagagcaaatcg

SEQ ID NO: 111 - DNA, 53 bp, synthetic construct
  source 1..53: mol_type = other DNA
gcatgcaaatcattacgaccgagattgccggcacgccacagaaacctcagaat

SEQ ID NO: 112 - DNA, 42 bp, synthetic construct
  source 1..42: mol_type = other DNA
gggcgaattgggtaccgggccccatatggcgtatccaagccg

SEQ ID NO: 113 - DNA, 47 bp, synthetic construct
  source 1..47: mol_type = other DNA
cactaaagggaacaaaagctggagctccgccacagaaacctcagaat

SEQ ID NO: 114 - DNA, 60 bp, synthetic construct
  source 1..60: mol_type = other DNA
gccctgcaggtcactattaatgatggtgatggtggtgacccaaagtgaactttggcttag

SEQ ID NO: 115 - DNA, 35 bp, synthetic construct
  source 1..35: mol_type = other DNA
gatttgctctcaatccgccctgcaggtcactatta

SEQ ID NO: 116 - DNA, 43 bp, synthetic construct
  source 1..43: mol_type = other DNA
cctcacgaagaagttaagcttgagcatcggaccgaagcataag

SEQ ID NO: 117 - DNA, 55 bp, synthetic construct
  source 1..55: mol_type = other DNA
attacttcatgagataaattaacctgcaggcgtataaacaatgcatactttgtac

(19) United States
(12) Patent Application Publication
(10) Pub. No.: US 2025/0297266 A1
(43) Pub. Date: Sep. 25, 2025

(54) METHODS FOR GENE AMPLIFICATION

(71) Applicant: The University of Queensland, St. Lucia, Queensland (AU)

(72) Inventors: Bingyin Peng, St. Lucia, Queensland (AU); Claudia Vickers, St. Lucia, Queensland (AU)

(21) Appl. No.: 18/849,098

(22) PCT Filed: Mar. 21, 2023
(86) PCT No.: PCT/AU23/50204

§ 371 Date: Sep. 20, 2024

(30) Foreign Application Priority Data
Mar. 21, 2022 (AU) 2022900699
Apr. 26, 2022 (AU) 2022901094

(51) Int. Cl. C12N 15/67; C12N 1/16; C12N 15/81; C12N 15/90; C12R 1/645

(52) U.S. Cl. CPC C12N 15/67; C12N 1/165; C12N 15/81; C12N 15/905; C12N 2820/704; C12R 2001/645

(57) ABSTRACT

Disclosed are methods of genetic engineering to manipulate gene copy number in vivo, as well genetic constructs for amplifying gene copy number in vivo, and recombinant cells that comprise amplified genes. The methods of increasing gene copy number involve reducing expression levels of a haploinsufficient gene in the genome of recombinant cells, such as through replacing the endogenous promoter with a weaker promoter.

### RELATED APPLICATIONS

This application claims priority to Australian Provisional Application No. 2022900699 entitled “Methods for gene amplification” filed 21 Mar. 2022 and Australian provisional patent application no. 2022901094 filed 26 Apr. 2022, the contents of which are incorporated herein by reference in their entirety.

### FIELD

This disclosure relates generally to methods of genetic engineering to manipulate gene copy number in vivo. The present disclosure also relates to genetic constructs for amplifying gene copy number in vivo, and recombinant cells that comprise amplified genes.

### BACKGROUND

All references, including any patent or patent application cited in this specification are hereby incorporated by reference to enable full understanding of the present disclosure. Nevertheless, such references are not to be read as constituting an admission that any of these documents forms part of the common general knowledge in the art, in Australia or in any other country.

To achieve economically viable yields and titers for any given gene or expression product in cell factories (bio-engineered cells for the biosynthesis of products of industrial interest), it is commonly necessary to increase or maximize expression of introduced genetic constructs. This is typically achieved by manipulating transcription levels of the polynucleotide encoding the desired product, via transcriptional control elements (promoters and other genetic sequences). However, this approach is often still insufficient or inefficient for a desired application (e.g. a strong promoter may still be incapable of the level of activity required for economically viable yields). Where particularly large amounts of product is required (e.g., in protein production systems), higher expression levels per cell can deliver a direct economic advantage to the bioprocess.

Increasing gene dosage/gene copy number can be used to improve expression levels; however, previously available methods for introducing multiple gene copies or amplifying gene number suffer from various drawbacks, such as genetic instability of amplified genetic material, or the requirement for exogenous selection systems, which can impact host cell fitness and/or impose further economic costs. Further, in the case where multiple gene copies are integrated at multiple random loci in the host genome, it renders downstream genetic manipulation of the cell (e.g., removal of the integrated copies or further addition of other genetic elements) more challenging and unpredictable.

Yeast, bacterial, archaean, fungal, algal, microalgae, cyanobacterial, insect and mammalian cells are currently being used as cell factories for the industrial production of biofuels, proteins, chemicals, and biopharmaceuticals. Bacterial, archaean, insect and mammalian cells have been used to produce biopharmaceuticals such as antibiotics, antibodies, enzymes, amino acids and peptides and other chemicals. Algae and microalgae are cultivated for biomass production, wastewater treatment, carbon dioxide fixation, synthesis of chemicals, fertilizers, bioplastics, and for the production of biopharmaceuticals, biofuels, and food ingredients such as fatty acids, amino acids, food flavoring or coloring. Industrial applications for cyanobacteria include biofuel production, nitrogen and carbon fixation, as well as synthesis of biopharmaceuticals and nutritional products. Brewer's yeast, Saccharomyces cerevisiae, is an important model organism for studying genome architecture, evolution and genetic engineering. It is also a valuable industrial microorganism. In yeast, yeast episomal plasmids (YEps) with auxotrophic/antibiotic markers or intended for genome integration into rDNA sites are typically used to increase gene dosage of a desired exogenous gene, but this approach is not stable in the absence of selection pressure. The requirement for such selection systems in industrial processes adds additional costs and often is not scalable. To stabilize strains without the need for antibiotic or auxotrophy systems, auto-selection markers such as glycolytic genes (FBA1, fructose-bisphosphate aldolase; POT1/TPI1, triosephosphate isomerase) can be used. However, this can add further complexity to the engineering of these strains.

Therefore, there is a need for alternative methods for producing high product yields in cell factory systems.

### SUMMARY

The present disclosure is predicated, at least in part, on the surprising finding that the evolutionary force and selection pressure exerted by a haploinsufficient gene can be exploited to drive gene amplification and maintenance. The Inventors have developed an in vivo gene amplification system to introduce multiple gene copies into a cell with mitotic stability. This can be achieved in a number of ways, as described herein.

Haploinsufficiency describes a state whereby one allele at a heterozygous locus provides little or no product, and the combined product from both alleles is insufficient to deliver the wild type phenotype. The expression of haploinsufficient genes is linked tightly to the growth fitness in many organisms, including yeast. In yeast, tandem amplification of fitness-associated genes permits improved fitness: e.g., amplification of xylose isomerase gene over the prolonged adaptive cultivation on xylose, amplification of cellubiose-utilizing genes over the prolonged adaptive cultivation on cellubiose, CUP1 amplification for enhanced resistance to copper ions, and the amplification of tandem repeated ribosomal DNA under some conditions. That is, when the expression level of a gene product is tightly linked to growth fitness, gene amplification evolves to meet the need for maximum growth.

Methods are disclosed herein that exploit the evolutionary force and selection pressure of a haploinsufficient gene, by reducing expression of the haploinsufficient gene to drive an increase in the copy number of the haploinsufficient gene (i.e., gene amplification). Also disclosed herein are methods that exploit the evolutionary force and selection pressure of a haploinsufficient gene, by reducing expression of the haploinsufficient gene to drive an increase in its copy number and ‘bystander’ amplification and maintenance of an operably connected heterologous nucleic acid. Methods of genetically modifying yeast are also disclosed herein for improving production of terpenes and proteins of interest. In illustrative examples disclosed herein, three products: sesquiterpene nerolidol, monoterpene limonene, and tetraterpene lycopene; limonene titer reached to ˜ 1 g L⁻¹ in the flask cultivation on 20 g L⁻¹ glucose, the highest reported titer in microbes under similar conditions. Additionally, yeast cells modified according to the present disclosure were found to express heterologous proteins to a level often observed in Escherichia coli systems.

Accordingly, in one aspect, a method is disclosed herein for increasing copy number of a haploinsufficient gene in the genome of a cell, the method comprising, consisting or consisting essentially of reducing expression of the haploinsufficient gene to thereby increase the copy number of the haploinsufficient gene in the genome of the cell.

In some embodiments, the haploinsufficient gene is operably connected to an origin of replication.

In another aspect disclosed herein, there is provided a method for increasing copy number of a heterologous nucleic acid sequence in the genome of a cell, the method comprising, consisting or consisting essentially of: introducing the heterologous nucleic acid sequence into the genome, wherein the heterologous nucleic acid sequence is introduced in operable connection with a haploinsufficient gene of the genome; and reducing expression of the haploinsufficient gene, wherein the reduced expression of the haploinsufficient gene increases copy number in the genome of a nucleic acid construct comprising the heterologous nucleic acid sequence and the haploinsufficient gene, thereby increasing the copy number of the heterologous nucleic acid sequence in the genome of the cell.

In some embodiments, the heterologous nucleic sequence comprises at least one coding sequence in operable connection with a promoter that is operable in the cell. In representative examples of this type, the heterologous nucleic sequence may be located upstream or downstream of the haploinsufficient gene.

In certain embodiments, the nucleic acid construct comprises an origin of replication.

The method may exclude rescuing expression of the haploinsufficient gene through use of a separate rescuing agent.

In specific embodiments, expression of the haploinsufficient gene is reduced by any one or more of the following: replacing the endogenous promoter of the haploinsufficient gene with a weaker promoter; replacing at least one codon of the haploinsufficient gene with a codon that has a lower translational efficiency in the cell than the codon it replaces and/or; adding at least one codon into the coding sequence of the haploinsufficient gene wherein the codon has a lower translational efficiency than other codons of the coding sequence; disrupting the haploinsufficient gene; modifying the haploinsufficient gene to include a nucleotide sequence encoding an RNA destabilizing element; and expressing a nucleic acid molecule in the cell, which reduces the level of an expression product of the haploinsufficient gene. A codon that replaces a codon of the haploinsufficient gene and a codon that is added to the coding sequence of the haploinsufficient gene are collectively referred to herein as a “codon that has a lower translational efficiency”.

In some embodiments, the resulting copy number of the nucleic acid construct is 2 to 200 copies, suitably 3 to 100 copies, suitably 3 to 70 copies, suitably 3 to 60 copies.

The cell may be a yeast, fungal, algal, microalgae, cyanobacterial, bacterial, insect or mammalian cell. In a preferred embodiment, the cell is a yeast cell.

In some embodiments, the haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11.

In some embodiments, the expression of the haploinsufficient gene is reduced by replacing the endogenous promoter of the haploinsufficient gene with a weaker promoter (i.e., a promoter that is weaker than the endogenous promoter of the haploinsufficient gene). In representative examples, the weaker promoter is selected from the group consisting of ERG1 promoter, PDA1 promoter, BTS1 promoter, GLO2 promoter and COG7 promoter.

In some embodiments, the haploinsufficient gene is operably connected to an origin of replication, wherein the origin of replication is ARS306 or ARS1max.

Disclosed herein in yet another aspect is a nucleic acid construct comprising a recombinant polynucleotide that reduces expression of a haploinsufficient gene in a cell of interest, wherein the haploinsufficient gene is endogenous to the cell.

In certain embodiments, the nucleic acid construct further comprises a heterologous nucleic acid sequence in operable connection with the haploinsufficient gene. The heterologous nucleic sequence may comprise at least one coding sequence in operable connection with a promoter that is operable in the cell. The heterologous nucleic sequence may be located upstream or downstream of the recombinant polynucleotide.

In some embodiments, the nucleic acid construct further comprises an origin of replication.

In an embodiment, the recombinant polynucleotide of the nucleic acid construct is selected from:
a. a polynucleotide that comprises a promoter that is weaker than the endogenous promoter of the endogenous haploinsufficient gene, which when introduced into the genome of the cell, is operably connected to the haploinsufficient gene;
b. a modified haploinsufficient gene that is distinguished from the endogenous haploinsufficient gene by replacement of the endogenous promoter of the endogenous haploinsufficient gene with a weaker promoter;
c. a modified haploinsufficient gene that is distinguished from the endogenous haploinsufficient gene by replacement of at least one codon of the haploinsufficient gene with a codon that has a lower translational efficiency in the cell than the codon it replaces:
d. a modified haploinsufficient gene that is distinguished from the endogenous haploinsufficient gene by disruption of endogenous haploinsufficient gene;
e. a modified haploinsufficient gene that is distinguished from the endogenous haploinsufficient gene by operably connecting a nucleotide sequence encoding an RNA destabilizing element to the endogenous haploinsufficient gene; and
f. a polynucleotide that reduces the level of an expression product of the haploinsufficient gene.

In embodiments in which the recombinant polynucleotide comprises a modified haploinsufficient gene that is distinguished from the endogenous haploinsufficient gene by replacement of the endogenous promoter of the endogenous haploinsufficient gene with a weaker promoter, the weaker promoter is suitably selected from the group consisting of ERG1 promoter, PDA1 promoter, BTS1 promoter, GLO2 promoter and COG7 promoter.

In some embodiments, the haploinsufficient gene is a gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11.

In certain embodiments, the origin of replication of the nucleic acid construct is an autonomous replicating sequence, wherein the autonomous replicating sequence is ARS306 or ARS1max.

In some embodiments, the nucleic acid construct comprises a coding sequence that encodes an expression product selected from a polypeptide (e.g. a polypeptide for producing a terpenoid, flavonoid or fatty acid, an antibody, a nanobody, etc.) or a functional RNA molecule (e.g., RNAi that inhibits expression of a target gene).

In still another aspect, a cell is disclosed that comprises a nucleic acid construct as broadly described above and elsewhere herein. The cell may be a yeast, bacterial, fungal, algal, microalgae, cyanobacterial, insect or mammalian cell. In a preferred embodiment, the cell is a yeast cell. In representative examples, the cell may comprise 2 to 200 copies, suitably 3 to 100 copies, suitably 3 to 70 copies, suitably 3 to 60 copies of the nucleic acid construct.

Disclosed herein in a further aspect is a method for expressing nucleic acid, the method comprising culturing a cell as broadly described above and elsewhere herein to express a nucleic acid construct as broadly described above and elsewhere herein.

In one aspect, the present disclosure provides a genetically modified yeast cell, comprising a nucleic acid construct in its genome, wherein the nucleic acid construct comprises: (1) a recombinant polynucleotide that reduces expression of a haploinsufficient gene that is endogenous to the cell of interest; (2) a heterologous nucleic acid sequence in operable connection with the haploinsufficient gene, wherein the heterologous nucleic sequence comprises at least one coding sequence in operable connection with a promoter that is operable in the cell; and (3) optionally an origin of replication. In certain embodiments: the recombinant polynucleotide is selected from (a) to (f) above, wherein the haploinsufficient gene is ribosomal 60S subunit protein L25 or GTPase-activating protein SEC23; the weaker promoter is selected from the group consisting of ERG1 promoter, PDA1 promoter, BTS1 promoter, GLO2 promoter and COG7 promoter; and the origin of replication is the autonomous replicating sequence ARS306 or ARS1max.

### BRIEF DESCRIPTION OF THE DRAWINGS

Embodiments of the disclosure are described herein, by way of non-limiting example only, with reference to the following drawings.

FIG. 1 shows the natural genome structures at the rDNA locus on chromosome XII and the CUP1 locus on chromosome VII (a) and design of the genetic construct design for in vivo gene amplification (HapAmp) (b). Autonomous replicating sequence (ARS). Arm 1 and Arm 2 are recombination arms/homologous arms for the integration of the construct into genome. Arm 3 are recombination arms/homologous arms functioning for in vivo gene amplification. The tandem amplified region (TAR) will comprise 1 or more copies of the gene of interest linked with the attenuated haploinsufficient (HIS) gene.

FIG. 2 shows changes in level of expression product when a selection of different promoters are used. Yeast enhanced green fluorescent protein (yEGFP) is used as the reporter in the cells at the exponential growth phase (EXP) and the post-diauxiediauxic shift growth phase (ETH) when ethanol is used as the carbon source. Yeast cells were grown in microplates and yEGFP fluorescence is expressed as percentage of exponential-phase auto-fluorescence of the reference strain. Mean values±standard deviations are shown (N≥2).

FIG. 3 shows design and characterization of gene amplification constructs for haploinsufficient target genes RPL25 or SEC23. A schematic of gene amplification constructs is shown in (a); maximum growth rate, yEGFP copy number, and yEGFP fluorescence in strains transformed with the constructs in (a) is shown in (b), (c), (e) respectively. Promoter characterization using yEGF) as the reporter in the cells at the exponential growth phase (EXP) and the post-diauxic-shift growth phase (ETH) when ethanol was used as the carbon source (d). yEGFP fluorescence is expressed as percentage of exponential-phase auto-fluorescence of the reference strain. Transformation plates of the yeast transformed with the constructs are shown in (f). Stability of the strain expressing EGFP via P_{BTS1}-RPL25 HapAmp construct is shown in (g). GFP fluorescence levels and population homogeneity did not change, for at least 48 generations, indicating genetic stability. Mean values±standard deviations are shown (N≥3 independent biological replicates).

FIG. 4 shows the genome structure at YOL127W (RPL25) locus in strain G3AG5 (Construct 3, FIG. 2); alignment with trimmed minION reads outputted by Canu assembler. Strain G3AG5 is deposited with Bioproject: PRJNA688119, under accession number SRR13774413.

FIG. 5 shows the genome structure at YOL127W (RPL25) locus in strain G3AA5 (Construct 4, FIG. 2) (b); alignment with trimmed minION reads outputted by Canu assembler, confirming that the constructs were integrated into the RPL25 (YOL127W) locus and that yEGFP-RPL25 sequences were amplified in tandem repeat structures. Strain G3AA5 is deposited with Bioproject: PRJNA688119, under accession number SRR13774412.

FIG. 6 shows characterization of nerolidol-producing strains, harboring nerolidol synthetic genes on a 2μ plasmid (N401-1) or integrated at amplified RPL25 locus (N401-2, N401-3, and N401-4). A schematic map of genetic vectors used to introduce nerolidol synthetic genes into yeast (a) & (b). In (c)-(h), strain characterization in two-phase flask cultivation with 20 g L⁻¹ glucose and dodecane overlay is shown. Y-FAST fluorescence was measured after 4-hydroxy-3-methylbenzylidene rhodanine (HMBR; final concentration 20 μM) was added to the yeast samples before flow cytometry assay, and is expressed as fold-change of exponential-phase auto-fluorescence of the reference strain GH4. Mean values±standard deviations are shown (c-f, h; N=4 independent biological replicates). Two-tailed Welch's t-test was used for comparing two groups, and p values were shown in (d) & (h).

FIG. 7 shows characterization of limonene-producing strains with limonene synthetic genes in a 2μ plasmid (LIM141R and LIM141R2) integrated at amplified RPL25 locus. A schematic map of genetic vectors used to introduce limonene synthetic genes into yeast is shown in (a). Strain characterization in two-phase flask cultivation with 20 g L⁻¹ glucose and dodecane overlay is shown in (b-f). Synthetic auxin 1-Naphthaleneacetic acid (NAA) was added to 1 mM at the late exponential growth phase (OD>4). Y-FAST fluorescence was measured after 4-hydroxy-3-methylbenzylidene rhodanine (HMBR) with final concentration 20 μM was added to the yeast samples before flow cytometry assay and is expressed as fold-change of exponential-phase auto-fluorescence of the reference strain GH4³⁰. Limonene and geraniol production at 96 hour was shown. Mean values±standard deviations are shown (b-f: N=3 or 4 independent biological replicates for LIM141R, LIM141M and LIM141MH; 3 independent cultures for LIM141R2).

FIG. 8 shows characterization of lycopene-producing strains with lycopene synthetic genes integrated at amplified RPL25 locus. Schematic maps of genetic vectors used to introduce lycopene synthetic genes into yeast (a). Lycopene production in flask cultivation is shown in (b). Yeast cells in exponential growth was inoculated into 20 mL MES-buffered YNB medium with 20 g L⁻¹ glucose in 125 mL Erlenmeyer flask to start a culture at OD600=0.2. Mean values±standard deviations are shown (N=4 independent biological replicates).

FIG. 9 shows characterization of the expression of heterologous proteins (AeBlue and HPV16 capsid L1) via multi-copy genome integration (MI) using PBTS1-RPL25-driven in vivo gene amplification. Schematic maps of genetic vectors used to express AeBlue and HPV16 L1 (a). Cells harboring an empty 2μ, the amplifiable AeBlue construct (MI), AeBlue-and-HPV16-L1 2μ plasmid, and amplifiable AeBlue-and-HPV16-L1 construct (MI) (b). Ultracentrifugation of the supernatant on an iodixanol gradient used to separate a band containing HPV16-L1 virus-like particles (shown by orange arrow), TEM confirming the presence of HPV16-L1 virus-like particles (VLPs) (sample labelled 4′ is a biological replicate of sample 4) (c). SDS-PAGE (sodium dodecyl sulphate-polyacrylamide gel electrophoresis) for whole cell lysates (d).

### DETAILED DESCRIPTION

### 1. Definitions

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described. For the purposes of the present disclosure, the following terms are defined below.

The present description uses numerical ranges to quantify certain parameters relating to this disclosure. It should be understood that when numerical ranges are provided, such ranges are to be construed as providing support for claim limitations that recite the lower value of the range as well as claim limitations that recite the upper value of the range. For example, a disclosed numerical range of 10 to 100 provides support for a claim reciting “greater than 10” (with no upper bounds) and a claim reciting “less than 100” (with no lower bounds) and provided support for and includes the end points of 10 and 100.

The articles “a” and “an” are used herein to refer to one or to more than one (i.e., to at least one) of the grammatical object of the article. By way of example, “an element” means one element or more than one element.

As used herein, the term “about” refers to a quantity, level, value, number, dimension, size, percentage or amount that varies by as much as 10% (e.g., by 10%, 9%, 8%, 7%, 6%, 5%, 4%, 3%, 2% or 1%) to a reference quantity, level, value, number, dimension, size, percentage or amount.

As used herein, the term “amplicon” refers to a piece of DNA or RNA that is the source and/or product of amplification or replication events.

The term “amplification” as used herein, for example in relation to gene amplification or transgene amplification, refers to an increase in copy number of a single copy gene or transgene to at least 2 copies. The increase in copy number is preferably 2 to 100 copies, preferably 2 to 90 copies, preferably 2 to 80 copies, preferably 2 to 70 copies, more preferably 2 to 60 copies, more preferably 4 to 60 copies, more preferably 4 to 50 copies, or any integer copy number between these ranges.

As used herein, “and/or” refers to and encompasses any and all possible combinations of one or more of the associated listed items, as well as the lack of combinations when interpreted in the alternative (or).

By “coding sequence” it is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “construct”, “nucleic acid construct” and the like refer to a recombinant genetic molecule including one or more nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present disclosure will generally include the necessary elements to direct expression of a nucleic acid sequence of interest that is also contained in the construct. Such elements may include control elements such as a promoter that is operably linked to (so as to direct transcription of) the nucleic acid sequence of interest, and often includes a polyadenylation sequence as well. In certain embodiments of the disclosure, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” (also referred to herein as an “expression cassette”) generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present disclosure, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

The term “corresponding” as used herein in reference to a particular gene is intended to mean an analogous or equivalent or comparable gene. For example, where reference is made to a corresponding endogenous gene, it is intended to mean the analogous, equivalent or comparable naturally-occurring gene. Where reference is made to a corresponding exogenous gene, it is intended to mean an analogous, equivalent or comparable exogenous gene. In some embodiments, the corresponding gene has analogous or equivalent function or having sequence similarity. In one embodiment, the corresponding gene may be identical in function and/or sequence. In another embodiment, the corresponding gene may have about the same function or activity. In another embodiment, the corresponding gene may have reduced function or activity. In some embodiments, the phrase “corresponds to” or “corresponding to” is meant a nucleic acid sequence that displays substantial sequence identity to a reference nucleic acid sequence. In general the nucleic acid sequence will display at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even up to 100% sequence identity to the reference nucleic acid sequence.

The terms “disruption” and “disrupted”, as applied to a nucleic acid, are used interchangeably herein to refer to any genetic modification that decreases or eliminates expression and/or the functional activity of the nucleic acid or an expression product thereof. For example, disruption of a gene includes within its scope any genetic modification that decreases or eliminates expression of the gene and/or the functional activity of a corresponding gene product (e.g., mRNA and/or protein). Genetic modifications include complete or partial inactivation, suppression, deletion, interruption, blockage, or down-regulation of a nucleic acid (e.g., a gene). Illustrative genetic modifications include, but are not limited to, gene knock-out, inactivation, mutation (e.g., insertion, deletion, point, or frameshift mutations that disrupt the expression or activity of the gene product), or use of inhibitory nucleic acids (e.g., inhibitory RNAs such as sense or antisense RNAs, molecules that mediate RNA interference such as siRNA, shRNA, miRNA; etc.), inhibitory polypeptides (e.g., antibodies, polypeptide-binding partners, dominant negative polypeptides, enzymes etc.) or any other molecule that inhibits the activity of a haploinsufficient gene or level or functional activity of an expression product of a haploinsufficient gene.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “endogenous” and “native” are used interchangeably herein to refer to a nucleic acid or protein, or part thereof, that is naturally present and/or expressed in an organism or cell thereof. For example, an “endogenous” haploinsufficient gene refers to a haploinsufficient gene that is naturally expressed in an organism or cell thereof. The term may also be used to refer to the naturally occurring genomic location of a given gene or genetic element of a particular organism. In contrast, the term “exogenous” refers to material or things such as polynucleotide or polypeptide sequences having an external origin, or is outside of an organism. A vector, plasmid, or other artificial construct that includes an endogenous polynucleotide sequence combined with polynucleotide sequences of the unmodified vector etc. is, as a whole, an exogenous polynucleotide and may also be referred to as an exogenous polynucleotide including an endogenous polynucleotide sequence. Also, a particular polynucleotide sequence that is isolated from a first organism and transferred to second organism by molecular biological techniques is typically considered an “exogenous” polynucleotide with respect to the second organism.

The term “expression”, as used herein, typically refers to any step involved in the production of an RNA molecule or a polypeptide, such as by transcription, post-transcriptional modification, translation, post-translational modification, and secretion.

The term “gene” is used herein to refer to a unit of inheritance that comprises a coding sequence and optionally transcriptional and/or translational regulatory sequences and/or non-translated sequences (i.e., introns, 5′ and 3′ untranslated sequences) whether or not such regulatory sequences are adjacent to coding and/or transcribed sequences. Accordingly, a gene may include or encode promoter sequences, signal peptides, terminators, translational regulatory sequences such as ribosome binding sites and internal ribosome entry sites, enhancers, silencers, insulators, boundary elements, replication origins, matrix attachment sites, and locus control regions. In some embodiments the gene may comprise only coding sequence. In other embodiments, the gene may comprise coding sequences and non-coding sequences.

The term “gene product” or “expression product” as used herein refers to an RNA or protein that results from expression of a gene. For example, the gene product may be an RNA, such as mRNA, rRNA, tRNA, miRNA or siRNA, or may be a polypeptide product.

As used herein, the term “haploinsufficiency” refers to a state in which the total level and/or activity of a gene product (e.g., a particular protein) is insufficient for normal cellular function. For example, haploinsufficiency arises where one allele at a heterozygous locus provides little or no gene product, and a single copy of the wild-type allele at a locus in heterozygous combination with a variant allele is insufficient for normal cellular function. In haploids, haploinsufficiency arises when a single copy of a gene is insufficient to maintain normal cellular function. A haploinsufficient gene is therefore a gene that needs more than one allele to be functional in order to maintain normal cell function or express the wild type phenotype, or when a single functional copy of a gene is insufficient to maintain normal cellular function. Consequently, haploinsufficient genes exhibit extreme sensitivity to decreased gene expression.

The term “homologous” is used herein in a comparative sense to indicate that a nucleotide or polypeptide sequence being referred to as having the same origin or structure.

The term “heterologous” is used herein in a comparative sense to indicate that a nucleotide or polypeptide sequence being referred to is from a different source, position or structure from the source or the origin, or is linked to a second nucleotide sequence (or polypeptide) with which it is not normally associated, or is modified such that it is in a form that is not normally associated with the original material. Therefore the term “heterologous nucleic acid sequence” is used herein to indicate a nucleic acid is from a different source, position or structure from the source or the origin, or is linked to a second nucleotide sequence (or polypeptide) with which it is not normally associated, or is modified such that it is in a form that is not normally associated with the original material. The term “heterologous nucleic acid sequence” is used interchangeably herein with the term “transgene”.

The term “homologous recombination” as used herein in relation to genetic manipulation and genetic engineering techniques, has the same meaning as would be understood by the person skilled in the art; that is, a method of introducing exogenous DNA sequences in a targeted controlled fashion, at a specific, pre-determined genomic region or loci. The pre-determined genomic loci will largely depend on the genomic region that is being targeted for integration of the polynucleotide construct.

The terms “mutant” and “variant” and “modified” may be used interchangeably herein, to refer to a non-wild-type organism, strain, expression pattern or expression level, gene/polynucleotide sequence or amino acid sequence. The terms “modification”, “alteration”, “substitution” and the like, as used herein in relation to an amino acid residue/position or a nucleotide, typically mean that the amino acid or nucleotide in the particular position has been modified compared to the amino acid of the wild-type or parent polypeptide.

As used herein, the term “nucleic acid”, “nucleic sequence”, “polynucleotide”, “oligonucleotide” and “nucleotide sequence” as used herein refers to mRNA, RNA, CRNA, rRNA, cDNA, or DNA, or a combination thereof. The term typically refers to polymeric form of nucleotides, either ribonucleotides or deoxynucleotides or a modified form of either type of nucleotide. The term includes single-, double- or triple-stranded forms of DNA and RNA. It can be of recombinant, artificial and/or synthetic origin and it can comprise modified nucleotides, comprising for example a modified bond, a modified purine or pyrimidine base, or a modified sugar. The nucleic acids of the present disclosure can be in isolated or purified form, and made, isolated and/or manipulated by techniques known per se in the art, e.g., cloning and expression of cDNA libraries, amplification, enzymatic synthesis or recombinant technology. The nucleic acids can also be synthesized in vitro by well-known chemical synthesis techniques, as described in, e.g., Belousov (1997) Nucleic Acids Res. 25:3440-3444.

As used herein, the term “operably connected” or “operably linked” refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence. Likewise, in the present disclosure, “operable connection” in a nucleic acid construct of a heterologous nucleic acid sequence with a recombinant polynucleotide that reduces expression of a haploinsufficient gene that is endogenous to a cell of interest, encompasses positioning and/or orientation of the heterologous nucleic acid sequence and haploinsufficient gene in such a way so that reduced expression of the haploinsufficient gene increases copy number in the genome of the nucleic acid construct.

The terms “origin of replication” and “replication origin” are used interchangeably to refer to a particular sequence or genomic location at which replication is initiated on a chromosome, genome, plasmid or virus.

The terms “peptide”, “polypeptide” and “protein” are to be understood as referring to a chain of amino acids linked by peptide bonds, irrespective of the number of amino acids forming said chain. Amino acids are typically represented by their one-letter or three-letters code, according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

A “promoter” refers to one or more a nucleic acid control sequences that direct transcription of a nucleic acid. As used herein, a promoter may include necessary nucleic acid sequences near the start site of transcription, such as, in the case of a polymerase II type promoter, a TATA element. A promoter may optionally include distal enhancer or repressor elements, which can be located as much as several thousand base pairs from the start site of transcription. “Promoter” includes a minimal promoter that is a short nucleic acid sequence comprised of a TATA-box and other sequences that serve to specify the site of transcription initiation, to which control elements (e.g., cis-acting elements) are added for control of expression. “Promoter” also refers to a nucleotide sequence that includes a minimal promoter plus control elements (e.g., cis-acting elements) that are capable of controlling the expression of a coding sequence or functional RNA. This type of promoter sequence consists of proximal and more distal upstream elements, the latter elements often referred to as enhancers. Accordingly, an “enhancer” is a nucleic acid sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific nucleic acid-binding proteins that mediate their effects. Promoters may be derived in their entirety from a native gene, or be composed of different elements derived from different promoters found in nature, or even be comprised of synthetic nucleic acid segments. A promoter may also contain nucleic acid sequences that are involved in the binding of protein factors which control the effectiveness of transcription initiation in response to physiological or developmental conditions. Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

The term “tandemly repeated amplicon” as used herein, refers to a stretch of nucleic acids that comprises two or more DNA amplicons that are repeated in such a way that the repeats lie adjacent or neighboring to each other.

The term “transgene” as used herein refers to any nucleotide sequence used in the transformation of an organism. Thus, a transgene can be a coding sequence, a non-coding sequence, a cDNA, a gene or fragment or portion thereof, a genomic sequence, a regulatory element and the like. A “transgenic” organism, such as a transgenic animal, transgenic plant, transgenic yeast, or transgenic bacterium, is an organism into which a transgene has been delivered or introduced and the transgene can be expressed in the transgenic organism to produce a product, the presence of which can impart an effect and/or a phenotype in the organism.

The term “vector” typically refers to a DNA or RNA molecule used as a vehicle to transfer recombinant genetic material, such as a heterologous nucleic acid construct of the present disclosure, into a host cell. The vector may be a linear or circular double stranded nucleic acid molecule. Suitable vectors include plasmids, bacteriophages, viruses, fosmids, cosmids, and artificial chromosomes. A vector typically comprises an insert (a heterologous nucleic acid sequence or transgene) and a larger sequence that serves as the “backbone” of the vector. The purpose of a vector which transfers genetic information to the host is typically to isolate, multiply, or express the insert in the target cell. Vectors can be episomal, i.e., do not integrate into the genome of a host cell, or can integrate into the host cell genome. The vectors may also be replication competent or replication-deficient. Exemplary polynucleotide vectors include, but are not limited to, plasmids, yeast artificial chromosomes (YACs), cosmids, transposons, synthetic DNA fragments. Exemplary viral vectors include, for example, AAV, lentiviral, retroviral, adenoviral, herpes viral and hepatitis viral vectors. Selection of the vectors to be used will take into consideration the size of the insert, the host cell to be transfected and the desired transformation efficiency or outcome, and would be readily known to the persons skilled in the art.

The term “recombinant”, as used herein, refer to a biomolecule, e.g., a gene or protein, or to a cell or microorganism. The term “recombinant” may be used in reference to cloned DNA isolates, chemically synthesized polynucleotides, or polynucleotides that are biologically synthesized by heterologous systems, as well as proteins or polypeptides encoded by such nucleic acids, e.g. enzymes. A “recombinant” nucleic acid is a nucleic acid linked to a nucleotide or polynucleotide to which it is not linked in nature. For example, the recombinant polynucleotide may be in the form of an expression vector. As use herein, a “recombinant cell” refers to a cell that has introduced into it exogenous nucleic acid, typically exogenous DNA, such as a vector or other polynucleotides. The term includes the progeny of the original cell into which the exogenous DNA has been introduced. Thus, a “recombinant cell” as used herein generally refers to a cell that has been transformed, transfected or transduced with exogenous DNA. The host cell may be transformed, transfected or transduced in a transient or stable manner. The exogenous nucleic acid is typically introduced into a host cell so that it is maintained as a chromosomal integrant or as a self-replicating extra-chromosomal vector. The term “recombinant cell” encompasses any progeny of a parent host cell that is not identical to the parent host cell due to the alterations introduced.

As used herein, “RNA destabilizing element” refers to a nucleic acid sequence in an RNA that is bound by proteins and which protein binding changes the stability and/or translation of the RNA. Examples of RNA destabilizing elements include Class I AU rich elements (ARE), Class II ARE, Class III ARE, U rich elements, GU rich elements, and stem-loop destabilizing elements (SLDE).

The term “sequence identity” as used herein refers to the extent that sequences are identical on a nucleotide-by-nucleotide basis or an amino acid-by-amino acid basis over a window of comparison (e.g. over 10, 15, 20, 30, 40, 50, 60, 70, 80, 90, 100, 120, 140, 160, 180, 200 or more nucleotides or amino acids residues). Thus, a “percentage of sequence identity” is calculated by comparing two optimally aligned sequences over the window of comparison, determining the number of positions at which the identical nucleic acid base (e.g., A, T, C, G) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present disclosure, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software. Sequences may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbi.nim.nih.gov/ or http://www.ebi.ac.uk/Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pair wise sequence alignment that creates an optimal global alignment of two sequences (e.g., using the Needleman-Wunsch algorithm).

In regard to the term “variants” and “derivatives”, these terms are taken to refer to a biological equivalent of the sequence from which it was derived.

The term “wild-type” is used herein to denote an organism, gene, or gene product, or the expression pattern or expression level of the gene or gene product in a non-modified organism; that is, as it appears in nature, or that which is most frequently observed in a population and is thus arbitrarily designed the “normal” or “wild-type” form.

Each embodiment described herein is to be applied mutatis mutandis to each and every embodiment unless specifically stated otherwise.

It is to be understood that this disclosure is not limited to the particular methodology, protocols, proteins, organisms, vectors, reagents etc. described herein as these may vary. It is also to be understood that the terminology used herein is for the purpose of describing particular embodiments only, and is not intended to limit the scope of the present disclosure that will be limited only by the appended claims. Unless defined otherwise, all technical and scientific terms used herein have the same meanings as commonly understood by one of ordinary skill in the art.

### 2. Methods for Increasing Copy Number of a Gene

The present disclosure provides a method for increasing copy number of a haploinsufficient gene in the genome of a cell. This method generally comprises, consists or consists essentially of reducing expression of the haploinsufficient gene to thereby increase the copy number of the haploinsufficient gene in the genome of the cell. Also provided is a method for increasing copy number of a heterologous nucleic acid sequence in the genome of a cell, driven by amplification (increasing the copy number) of an operably connected haploinsufficient gene.

Reducing the expression of the haploinsufficient gene product can be achieved in many ways. For example, the expression level of the of haploinsufficient gene product can be reduced by reducing the level of transcription and/or translation of the haploinsufficient gene. This may include means to reduce the rate of transcription or translation, or by reducing the number of transcripts or protein products produced from the haploinsufficient gene. This may include means that degrades, inactivates or destabilizes the haploinsufficient gene transcript or expression product as defined herein. For example, this may include the provision of siRNA, miRNA, an antisense DNA or antisense RNA molecules that ultimately results in a reduction in the level of the haploinsufficient gene product.

Reduced expression level provides an evolutionary and selection force that drives an increase in the copy number of the haploinsufficient gene, so that cells are viable, or maintain growth fitness. This selective pressure driving the increase in copy number of the haploinsufficient gene can be advantageously exploited to effect bystander amplification of an operably connected heterologous nucleic acid sequence. In other words, the evolutionary and selection force exerted by the haploinsufficient gene typically encompasses additional ‘bystander’ regions situated around or neighboring the haploinsufficient gene, resulting in concomitant increase in the copy number of neighboring sequences.

### 2.1 Haploinsufficient Genes

In mammals, about 300 genes are known to be haploinsufficient (Dang et al. Eur J Human Genet. 16(11): 1350-7), including IFNGR2 (Interferon gamma receptor 2), PTEN, BRCA1 and 2, and p53, TERC, and RUNX genes. In the yeast Saccharomyces cerevisiae, more than 180 haploinsufficient genes have been identified by fitness profiling of heterozygous deletion strains. Examples of haploinsufficient genes in yeast include: RPL25 (ribosomal 60S subunit protein L25), SEC23 (component of the Sec23p-Sec24p heterodimer of the COPII vesicle coat), RPL33A, RPS15, RPC10, RPS5, ACT1, NIP1, RPS13, NUS1, SMC1, RNA14, RPB7, SPC97, STH1, ARP7, TAF61, RPN11, YPL142C, SEC23, RPL18A, act1, RPL17A, nip1, rpb8, CCT7, CCT2, RPL5, RPS13, RPO26, YDL193W, YLR076C, RRP4, RPL30, RPS20, YBR190W, sui2, YNL313C, rpb5, smc1, RPB3, TUB1, RVB2, SEC34, CCT3, RNA14, YHR083W, NMD3, YPR136C, RRP45, rpb7, YHR196W, DYS1, SPC97, CCT4, RPS2, SUI3, TAF145, RRP9, TIF35, YDR449C, YNL110C, TIF6, TSC10, ndc1, RPS3, DIS3, esp1, prp11, YNL114C, NOG1, SMD2, CDC47, MEX67, YJL009W, RRP43, PAN1, CCT5, YHR085W, MTR3, IMP3, SIK1, YMR093W, SPC98, CFT2, YDR367W, TAF90, PAB1, MOB1, ENP1, SPT6, RPP0, RIM2, YDL221W, IMP4, YJL069C, YLR339C, ARP9, RPC53, YDR355C, YGL047W, YML093W, YCL053C, NOP1, UTR5, YGR115C, TID3, NSP1, YDL152W, RPT3, GCD10, SPB1, YDR365C, GNA1, SEC53, YIR010W, YML127W, DCP2, HXT12, ORC4, mcm2, RSC6, RPC11, TFB1, HYP2, YGR277C, GP18, TLG1, NUP145, YLR033W, RLP7, pol1, RPB10, RRP42, RPN5, YDR060W, YDR396W, GLC7, RPP1, SEC24, yef3, rpc19, rap1, RPN2, DNA43, DIP2, cdc25, CSL4, ACC1, NOP58, BFR2, YDR339C, spp41, ECO1, YIL083C, RHO3, SFH1, YNR046W, YOL022C, YOL134C, ipl1, ATP16, SEC31, YDR013W, FAL1, YRA1, YFR003C, SLN1, YKR071C, SEC14, SEC21, cdc13, BCP1, TRS120, YDR412W, YDR437W, PUP3, EPL1, TAF67, NHP2, YDL209C, STS1, SQT1, sec11, YKR081C, RFC4, YPL251W, MED8, tub2, PRE5, BRX1, YPL233W, MRS5, POP4, ses1, YFL035C, YGR128C, PUP2, PRI1, EXO70, YNL132W, rpc34, MAS6, ARC40, NUP192, SEC65, YNL038W, top2, alg1, RPN6, TIM22, TFC6, prp3, SKI6, YHR188C, ERG9, GCD14, kre9, NOP4, YBR070C, pgi1, YIL003W, NUP159, RPL15A, prp4, alg7, YDL015C, COP1, DAD1, SSS1, PCF11, YFL018W-A, ERG1, MET30, YJL011C, MTR4, NUP82, SMC4, HRT1, NAN1, SHR3, PDS1, YDR434W, PRE4, CRM1, DNA2, YLR243W, ROT1, POP3, SRB6, TRS20, rib5, rpo21, HEM3, DBF4, RSC8, ERG7, YHR186C, cdc6, RAM2, STU2, TUB4, YCS4, DBP9, TAF65, YNL026W, YNL260C, RPB11, pet9, YDL148C, YDR053W, SLU7, SRP101, FRQ1, YDR413C, cdc4, YPT1, YGR280C, ARP4, ARP3, YKL195W, GCD7, FOL3, Rsa2, fol1, MED7, NIP29, REB1, cdc53, YDL196W, GLE1, TRR1, NCB2, YDR527W, RRN7, YJL072C, NET1, PRP19, CDC46, sis1, SEC12, RPA43, rpa190, SRP68, PRE2, mak5, cdc2, SAS10, YPD1, HEM13, RRP1, YDR489W, pre1, FRS2, hip1, SEC6, YJL097W, YLR002C, PIK1, CDC33, ORC2, EXO84, YFH1, ARH1, TFB3, SPC105, TOM20, YIL104C, TAO3, TRL1, MPP10, GRC3, YLR022C, STT4, RPM2, LST8, sec2, PRE6, RER2, PDI1, cdc7, KRS1, DOP1, TRS31, rib3, YGR265W, YHR070W, YRB2, PRE3, SMC3, YJL195C, YLR101C, YLR323C, AFG2, MPT1, YNL247W, RFC3, cdc31, idi1, spt14, SEC8, rib7, cdc28, RPT2, kin28, LCB2, pdc2, SMT3, YDR531W, CBF2, fol2, cdc12, PRP21, DRS1, BOS1, TAF19, NUF2, YOL146W, pup1, YTM1, PRE7, AME1, YDL016C, YRB1, RVB1, RPN9, SNM1, PMI40, RPT6, UFD1, ZPR1, cdc8, ACP1, YKR038C, YKR079C, YLR007W, TOM22, YNL306W, YOL078W, RIO1, prt1, NUD1, rad53, RPL32, ira1, sup45, NFS1, PGK1, SRP14, SNU23, GUK1, YGR190C, RRP3, QNS1, BIG1, YJL091C, HYS2, YLL034C, YSH1, YML125C, YNL245C, TBF1, STN1, WBP1, YGR156W, TYS1, gpi1, YJL010C, YJL086C, YKL059C, ECM9, RRN5, ADE13, SEC61, YML023C, ERG13, YNL124W, sui1, DBP6, RPO31, RPT5, MYO2, ALA1, SEC62, SRP72, MYO1, MLC1, and MYO2. Further examples of haploinsufficiency genes have been described elsewhere (see for example, Deutschbauer et al. (2005) Genetics 169:1915-1925). In some embodiments of the disclosure, the haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11. In one embodiment of the disclosure, the haploinsufficient gene is RPL25. In another embodiment of the disclosure, the haploinsufficient gene is SEC23.

Haploinsufficient genes can also be identified by comparative genomics and their suitability confirmed by testing growth fitness in association with expression dosage of a gene. Means and method for identifying haploinsufficient genes would be known to the persons skilled in the art. For diploid organisms, haploinsufficiency can also be achieved by disrupting one allele and integrating the amplifiable nucleic acid construct at the other allele locus, or by simultaneously integrating the amplifiable constructs at both alleles, to give rise to reduced gene dosage of the haploinsufficient gene. Established genetic recombination or genetic engineering techniques can be used for targeted allele disruption and integration of genetic construct. For example, site directed mutagenesis for targeted allele disruption, and nuclease-mediated DNA double-chain break like CRISPR systems for the integration of the amplifiable construct.

### 2.2 Reducing the Level of the Haploinsufficient Gene Product

Reducing the expression of the haploinsufficient gene can be achieved in many ways. For example, expression of the haploinsufficient gene can be reduced by reducing the transcription and/or translational efficiency of the haploinsufficient gene.

Alternatively, or in addition, the expression of the haploinsufficient gene product may be reduced by replacing the endogenous promoter of an endogenous haploinsufficient gene with a weaker promoter. The weaker promoter as described herein is to be understood in a comparative sense; that is the, the weaker promoter controlling the expression of the haploinsufficient gene is weaker relative to the native or endogenous promoter of the haploinsufficient gene. Driving expression through a weaker promoter attenuates the transcription level of the haploinsufficient gene.

Alternatively, or in addition, the level of the haploinsufficient gene product is reduced by modulating transcriptional and/or translational activity (i.e. rate of transcription, or production of mRNA) through the use of non-preferred codons (i.e., codons that have a lower transcriptional and/or translation efficiency than the codons they replace), whereby for example, replacement or addition of one or more codons in the haploinsufficient gene coding sequence with alternative codons that have a lower transcriptional and/or transcriptional efficiency functions to reduce the expression of the haploinsufficient gene.

In some embodiments, the level of the haploinsufficient gene product is reduced by driving expression of the haploinsufficient gene through a weaker promoter and the use of a variant haploinsufficient gene comprising non-preferred codons.

Expression of the haploinsufficient gene may also be reduced through disruption of the haploinsufficient gene. For example, the haploinsufficient gene may be disrupted by means that degrades, inactivates or destabilizes the haploinsufficient gene transcript or expression product as defined herein. For example, this may include the provision or expression of siRNA, miRNA, an antisense DNA or antisense RNA molecules that results in reduced expression of the haploinsufficient gene. Reducing expression of the haploinsufficient gene product can comprise modifying the haploinsufficient gene to include a nucleotide sequence encoding an RNA destabilizing element.

Disrupting the haploinsufficient gene may include replacing the endogenous gene with a variant haploinsufficient gene that has reduced expression and/or function. This variant haploinsufficient gene may comprise mutations that affect gene function, or comprise protein degradation motifs. This may include the modification of the haploinsufficient gene to include ubiquitin molecules that targets the expression product for degradation. For example, the haploinsufficient gene may be modified to include synthetic protease sites that results in targeted protein degradation, which ultimately results in a reduction in the level of the haploinsufficient gene product.

### 2.3 Weaker Promoter

In some embodiments, the expression of the haploinsufficient gene product is reduced by modulating transcriptional activity (i.e. rate of transcription, or production of mRNA) by replacing the endogenous promoter of the haploinsufficient gene with a weaker promoter.

The identification of suitable weaker promoters must be determined relative to the endogenous promoter of the native haploinsufficient gene. Standard methods of testing and assays for comparing promoter strength using reporter gene assays, including those disclosed herein, will be known to persons skilled in the art. By the way of an example, promoters that have been shown to drive a range of expression levels include promoters of RPL33A, RPS15, RPC10, ACT1, NIP1, RPS13, NUS1, SMC1, RNA14, RPB7, SPC97, STH1, ARP7 and TAF61 genes. The weak promoters can be from the promoters controlling the expression of a transcriptional factor, including GLN3, TOR1, DAL80, GCR1, GCR2, YNF1, YPK2, ADR1, NRG1, MIG1, ROX1, HAP4, HAC1, and UPC2 (Peng et al. Communication Biology). In one embodiment of the disclosure, the weaker promoter is selected from the ERG1 promoter, the PDA1 promoter, the BTS1 promoter, the GLO2 promoter, or the COG7 promoter as means of controlling expression of the haploinsufficient gene. Examples of promoter strength characterization will be known to be persons skilled in art, and have been previously disclosed, including in Peng et al. Microbial cell factories 14, 91 (2015).

The weak or weaker promoter can drive expression of the haploinsufficient gene at a level that is no more than 99% to 1% (and all integer percentages in between, including 95%, 90%, 80%, 70%, 60%, 50%, 40%, 30%, 20%, 10%, 5% 1%) or even less, of the level of the haploinsufficient gene driven by the native promoter.

The weaker promoter controlling the expression of the haploinsufficient gene may be 1-20 times weaker than the native or endogenous promoter. In other embodiments, the weaker promoter controlling the expression of the haploinsufficient gene is 1-10 times weaker than the native promoter. In other embodiments, the weaker promoter controlling the expression of the haploinsufficient gene is 2-8 times weaker than the native promoter. In other embodiments, the weaker promoter controlling the expression of the haploinsufficient gene is 2-5 times weaker than the native promoter. In other embodiments, the weak promoter controlling the expression of the haploinsufficient gene that is 2-4 times weaker than the native promoter. Standard methods for comparing and testing promoter strength using reporter gene assays in the host cell of interest can be easily performed by the skilled person. For example, the strength of the native promoter of the haploinsufficient gene in driving reporter gene expression can be compared to a range of known promoters to identify a promoter that is suitably weaker (i.e. comparing transcriptional efficiency/amount of transcript or polypeptide gene product produced). Non-preferred codons have lower translational efficiency.

Although exploitation of codon usage bias has been previously used to optimize translation, inclusion of non-optimal, less preferred or rare codons (collectively referred to herein as “non-preferred” codons) that have lower transcriptional and/or translational efficiency can also attenuate transcription and translation. Examples of non-preferred codons would be known to the person skilled in the art (e.g. Sharp et al. (1988) Nucleic Acids Research 16(17):8207; Athey et al. (2017) BMC Informatics 18:391). For example, in yeast, the non-preferred glycine codon GGA has lower translational efficiency. Codons with lower translational efficiency and codon usage bias for different organisms will be known to the person skilled in the art.

Thus, in some embodiments, the expression of the haploinsufficient gene product is reduced by replacing at least one codon of the haploinsufficient gene with a codon that has a lower transcriptional or translational efficiency in the cell, and/or by adding to the haploinsufficient gene at least one codon that has a lower transcriptional or translational efficiency in the cell. Non-preferred codon with lower transcriptional or translational efficiency can be added upstream or downstream of the gene (e.g., in an untranslated region of the gene), or within the coding sequence of the gene.

In some embodiments, 1, 2, 3, 4, 5 or more non-preferred codon(s) is (are) introduced into the haploinsufficient gene. In embodiments in which codons of the haploinsufficient gene are replaced with non-preferred codons, at least about 5%, 10%, 15%, 20%, 25%, 30%, 35%, 40%, 50%, 60%, 70%, 80%, 90%, 95%, 96%, 97%, 98%, 99% of the codons of the of the haploinsufficient gene may be replaced with non-preferred codons.

In some embodiments, introduction of the non-preferred codon does not result in a modification in the amino acid sequence of the haploinsufficient gene product. In other embodiments, the non-preferred codon that is introduced results in a modification in the amino acid sequence of the haploinsufficient gene product, to give rise to a variant polypeptide of the haploinsufficient gene product. The modification in the amino acid sequence of the haploinsufficient gene product maybe an amino acid insertion. The modification in the amino acid sequence of the haploinsufficient gene product may be an amino acid substitution. The modification in the amino acid sequence of the haploinsufficient gene product may be an amino acid deletion. It will be appreciated, that the modification in the amino acid sequence by incorporation of a non-preferred codon should not result in a non-functional haploinsufficient gene product. In some embodiments, the modification results in reduced expression of the haploinsufficient gene.

### 2.4 Bystander Amplification

Without wishing to be bound by any one theory or mode of operation, it is proposed that genetic manipulations that lead to reduced expression of a haploinsufficient gene result in selective pressure that drives an increase in the copy number of the haploinsufficient gene to maintain growth fitness of the cell. In accordance with the present disclosure, this increase in copy number not only amplifies the haploinsufficient gene but extends to neighboring genomic regions upstream or downstream of the haploinsufficient gene, which are referred to herein as ‘bystander’ regions. This phenomenon can be exploited advantageously to effect bystander amplification of any heterologous nucleic acid sequences or transgenes that are situated adjacent and operably connected to the haploinsufficient gene.

The heterologous nucleic acid sequence can be positioned at any suitable position relative to the haploinsufficiency gene, which permits bystander amplification of the heterologous nucleic acid sequence when the genetically manipulated haploinsufficient gene is amplified. Such positioning can be determined through routine procedures known in the art. In representative examples, the heterologous nucleic acid sequence may be separated from the haploinsufficient gene by about 1 to about 4000 bp (and all integer base pairs in between), by about 1 to about 2000 bp (and all integer base pairs in between), by about 1 to about 1000 bp (and all integer base pairs in between), by about 1 to about 500 bp (and all integer base pairs in between), by about 1 to about 300 bp (and all integer base pairs in between), by about 1 to about 200 bp (and all integer base pairs in between), or by about 1 to about 100 bp (and all integer base pairs in between). In some embodiments, the heterologous nucleic acid sequence may be separated from the haploinsufficient gene by no more than 10 bp, 20 bp, 30 bp, 40 bp, 50 bp, 60 bp, 70 bp, 80 bp, 90 bp, 100 bp, 150 bp, 200 bp, 250 bp or 300 bp. The skilled person would also understand that the distance the heterologous nucleic acid sequence is separated from the haploinsufficient gene may be influenced by the size of the heterologous nucleic acid sequence that flanks the haploinsufficient gene, but this is well within the ordinary skill in the art.

Expression of the haploinsufficient gene may also be reduced by targeted modification. For example, the haploinsufficient gene may be modified by disrupting the endogenous haploinsufficient gene (e.g., by knock-out) and integrating an exogenous haploinsufficient gene into the genome, wherein the exogenous haploinsufficient gene is expressed at a lower level than the endogenous haploinsufficient gene before disruption.

Disruption of the haploinsufficient gene can be achieved by deleting the endogenous haploinsufficient gene. The entire haploinsufficient gene, or only part of the gene can be deleted, so that the haploinsufficient gene is no longer functional; and an exogenous haploinsufficient gene can be integrated into the genome, wherein the exogenous haploinsufficient gene is expressed at a lower level than the endogenous haploinsufficient gene before disruption. Alternatively, the haploinsufficient gene can be disrupted by insertion of an exogenous sequence into the haploinsufficient gene, resulting in gene inactivation, either by producing a non-functional gene product, or by targeting the gene product for destruction or silencing; for example, the introduction of a stop codon, retrotransposons, anti-sense sequences, or siRNA sequences.

The haploinsufficient gene knock out strategies can be achieved using gene targeting strategies such as homologous recombination. The knock-out strategies may also be targeted at pre-determined, or a specified genome location using other targeted, site-specific genome integration strategies such as CRISPR-Cas9, Zinc Finger nucleases and TALEN genome editing techniques, application of which would be known to the person skilled in the art.

Insertion of the nucleic acid construct can be targeted to a pre-determined, or a specified genome locus. Methods of targeted, site-specific genome integration include using homologous recombination and CRISPR-Cas9, Zinc Finger nucleases and TALEN genome editing techniques, application of which would be known to the person skilled in the art. The nucleic acid construct can be targeted to the endogenous genomic location of the haploinsufficient gene, such that integration of the nucleic acid construct results in substitution of the native promoter of the haploinsufficient gene with the weaker promoter. Alternatively, the nucleic acid construct is targeted to the endogenous genomic location of the haploinsufficient gene, such that integration results in substitution of the entire endogenous haploinsufficient gene.

In another scenario, the endogenous haploinsufficient gene is disrupted and the nucleic acid construct comprising an exogenous haploinsufficient gene that is expressed at a lower level than the endogenous haploinsufficient gene before disruption, can be targeted for integration at a genomic location away from the endogenous haploinsufficient gene, or can be randomly integrated (i.e. not targeted to a specific genomic location).

In methods where the reducing the expression of the haploinsufficient gene comprises replacing the endogenous promoter of the haploinsufficient gene with a weaker promoter, or replacing or adding at least one codon of the haploinsufficient gene with a codon that has a lower translational efficiency in the cell, the integration of the polynucleotide construct is targeted. That is, the integration of the nucleic construct is targeted to the genomic loci comprising the endogenous promoter of the endogenous haploinsufficient gene or the endogenous haploinsufficient gene. The nucleic acid construct can be targeted for integration in the genome of the cell through homologous recombination, methods of which would be known to persons skilled in the art.

Targeting the genetic modifications, such as incorporation of non-preferred codons at a pre-determined, or a specified genome location can be performed using other targeted, site-specific genome integration strategies such as CRISPR-Cas9, Zinc Finger nucleases and TALEN genome editing techniques, application of which would be known to the person skilled in the art.

### 3. Nucleic Acid Constructs

Provided herein is a nucleic acid construct comprising a recombinant polynucleotide that reduces expression of a haploinsufficient gene that is endogenous to a cell of interest.

The nucleic acid construct, when introduced into the cell may be amplified in the cell to form a tandemly repeated amplicon in the genome of the cell. This tandemly amplified region comprises multiple copies of the nucleic acid construct.

The tandem repeated amplicon may contain 2-200 copies or repeats of the DNA segments or nucleic acid constructs. The tandem amplified region may contain 2 to 100 copies or repeats of the DNA segments or nucleic acid constructs. The tandem amplified region may contain 2 to 80 copies or repeats of the DNA segments or nucleic acid constructs. The tandem amplified region may contain 2 to 70 copies or repeats of the DNA segments or nucleic acid constructs. The tandem amplified region may contain 2 to 60 copies or repeats of the DNA segments of nucleic acid constructs, more preferably 4 to 60 copies or repeats of the DNA segments nucleic or acid constructs, more preferably 4 to 50 copies or repeats of the DNA segments nucleic or acid constructs, or any integer copies or repeats between these ranges.

In some embodiments, the nucleic acid construct further comprises a heterologous nucleic acid sequence in operable connection with the haploinsufficient gene.

The recombinant polynucleotides described herein may comprise a native sequence (e.g., an wild-type or native sequence that encodes a wild-type protein) of the haploinsufficient gene, or a variant, a derivative of the haploinsufficient gene, or a part or a fragment thereof of the haploinsufficient gene. Recombinant polynucleotide variants or derivatives may contain one or more substitutions, additions, deletions and/or insertions, as further described herein.

The polynucleotide variant may result in altered efficiency in transcriptional and translational regulation of the polynucleotide, such that the polynucleotide is capable of elevated or reduced expression. The polynucleotide variant may encode a polypeptide that has the amino acid sequence of the native or wild type polypeptide of the haploinsufficient gene. The polynucleotide may encode a polypeptide that has a variant polypeptide, such that the encoded polypeptide retains functional activity. The activity of the encoded polypeptide may be partially or substantially diminished relative to the unmodified or reference polypeptide. The activity of the encoded polypeptide may be partially or substantially augmented relative to the unmodified or reference polypeptide. The effect on the enzymatic activity of the encoded polypeptide may generally be assessed as described herein and known in the art.

The recombinant polynucleotide may comprise a polynucleotide that comprises a weaker promoter that has a lower transcriptional activity than the native promoter that is operably connected to the haploinsufficient gene such that when it is inserted upstream of the haploinsufficient gene, it will drive expression of the haploinsufficient gene at reduced levels when compared to the native promoter.

The nucleic acid construct of the present disclosure further comprises a heterologous nucleic acid sequence in operable connection with the haploinsufficient gene.

The heterologous nucleic acid sequence comprises at least one coding sequence in operable connection with a promoter that is operable in the cell. This allows expression of the coding sequence. The coding sequence can be a gene that encodes for a heterologous protein. The coding sequence can encode for heterologous gene products, which may be valuable in the industrial production of biofuels, proteins, biochemicals, chemicals, enzymes, pharmaceuticals and biopharmaceuticals. The coding sequence can encode for genes or polypeptides for producing products such as terpenoids, flavonoids, fatty acids, RNAi, nanobodies, phenolics, isoprenoids, alkaloids, and polyketides. Biopharmaceuticals include vaccines, insulin, antibodies, erythropoietin, hormones, blood factors, interferons, interleukins, growth factors, fusion proteins, recombinant enzymes. In some embodiments, the coding sequence encodes for sesquiterpene nerolidol, monoterpene limonene, or tetraterpene lycopene.

A nucleic acid construct as disclosed herein may comprise homologous arms for targeted homologous recombination mediated integration into the genome. Design (i.e., length, nucleotide sequence) of the homologous arms would be known to the persons skilled in the art. The homologous arms of the nucleic acid construct are situated flanking the heterologous nucleic acid sequence and the exogenous haploinsufficient gene.

The nucleic acid construct as disclosed herein may include an origin of replication that can be situated anywhere in the region between the homologous arms of the nucleic acid construct. The origin of replication may be situated adjacent to the heterologous nucleic acid sequence. The origin of replication may be situated adjacent to the haploinsufficient gene or portions thereof. The origin of replication may be situated between the heterologous nucleic acid sequence and haploinsufficient gene. The coding sequences and heterologous nucleic acid sequences described herein may be suitably deduced or derived from the amino acid sequence of the polypeptides described herein and codon usage may be adapted according to the host cell in which the nucleic acid shall be transcribed.

As will be understood by those skilled in the art, the nucleic acid constructs, the heterologous nucleic acids and coding sequences of this disclosure can include genomic sequences, extra-genomic, and plasmid-encoded sequences and smaller engineered gene segments that express, or may be adapted to express, proteins, polypeptides, peptides and the like. Such segments may be naturally isolated, or modified. Additional coding or non-coding sequences may, but need not, be present within a polynucleotide of the present disclosure, and a polynucleotide may, but need not, be linked or conjugated to other molecules and/or support materials.

The nucleic acid construct of the present disclosure can be up to about 10000 base pairs in length. The nucleic acid construct of the present disclosure can be up to about 9000 base pairs in length, up to about 8000 base pairs in length, up to about 7000 base pairs in length, up to about 6000 base pairs in length, up to about 5000 base pairs in length, up to about 4000 base pairs in length, up to about 3000 base pairs in length, up to about 2000 base pairs in length up to about 1000 base pairs in length, or from about 500 to about 10000 bases pairs in length (and all integer base pairs in between). The size of the nucleic acid construct that can be accommodated by a selected vector can be readily determined by the skilled person.

The heterologous nucleic acid sequences disclosed herein may be codon optimized to improve expression in the cell. Suitable methods for codon optimization will be familiar to persons skilled in the art, illustrative examples of which are described in the reference manual Sambrook et al. (Sambrook et al., 2001). Codon usage bias for different organisms will be known to the person skilled in the art.

### 3.1 Homologous Arms

The nucleic acid construct may further comprise homologous arms that facilitate targeted genomic integration. In some embodiments, replacement of the endogenous promoter or the endogenous haploinsufficient gene can be achieved by homologous recombination at a pre-determined genomic locus.

The homologous arms of the nucleic acid construct are homologous to DNA sequences of the host cell genome which are adjacent or flanking the targeted locus. The sequence of the homologous arms may be identical or similar (which include homologous identical sequences and homologous non-identical sequences) to the regions of the host cell genome to which the homologous arms are complementary. Homologous non-identical sequences refer to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. As used herein, the degree of homology between the two homologous, non-identical sequences is sufficient to allow homologous recombination there between, utilizing normal cellular mechanisms. Two homologous non-identical sequences can be any length and their degree of non-homology can be as small as a single nucleotide (e.g., for a genomic point mutation introduced targeted homologous recombination) or as large as 10 or more kilobases (e.g., for insertion of a gene at a predetermined locus in a chromosome). Two polynucleotides comprising homologous non-identical sequences need not be the same length. For example, an exogenous polynucleotide (i.e., vector polynucleotide) of between 20 and 4,000 nucleotides or nucleotide pairs can be used.

The characterization of two sequences as homologous, identical sequences or homologous, non-identical sequences may be determined by comparing the percent identity between the two sequences (polynucleotide or amino acid). Homologous, identical sequences have 100% sequence identity. Homologous, non-identical sequences may have sequence identity greater than 80%, greater than 85%, greater than 90%, greater than 91%, greater than 92%, greater than 93%, greater than 94%, greater than 95%, greater than 96%, greater than 97%, greater than 98%, or greater than 99%.

The homologous arms may be any length that allows for site-specific homologous recombination. A homologous arm may be any length between about 2000 bp and 500 bp including all integer values between. For example, a homologous arm may be about 2000 bp, about 1500 bp, about 1000 bp, or about 500 bp. In embodiments having two homologous arms, the homologous arms may be the same or different length. Thus, each of the two homologous arms may be any length between about 2000 bp and 500 bp including all integer values between. For example each of the two homologous arms may be about 2000 bp, about 1500 bp, about 1000 bp, or about 500 bp. A portion of the polynucleotide arm adjacent to one or both (i.e., between) homologous arms modifies the targeted locus in the host cell genome by homologous recombination. Techniques for homologous recombination in other organisms are generally known (see, e.g., Kriegler, 1990, Gene transfer and expression: a laboratory manual, Stockton Press). The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the host cell genome including coding regions, non-coding regions, and regulatory sequences. In an embodiment the modification may ablate a gene thereby creating a knock-out organism. In another embodiment, the modification may modulate the expression of the gene. In an embodiment the modification may add a gene that functions as a reporter or marker (e.g., GFP or antibiotic resistance). In an embodiment, the modification may add an exogenous gene. In an embodiment, the modification may add an endogenous gene under control of an exogenous promoter (e.g., a strong promoter, a weak promoter, an inducible promoter, etc.).

### 3.2 Origins of Replication

In some embodiments, the nucleic acid construct may include addition of exogenous protein domains including post-translational modification sites, protein-stabilizing domains, cellular localization signals, and protein-protein interaction domains. In other embodiments, the nucleic acid construct may comprise addition of nucleic acid sequences that are not translated into a protein including, but not limited to, a non-coding RNA molecule, a gene regulatory element, a promoter, a regulatory protein binding site, a RNA binding site, a ribosome binding site, a transcriptional terminator, or a RNA-stabilizing element. In an embodiment, the polynucleotide construct may include an origin of replication.

In eukaryotes, the origin of replication is where the hexameric protein complex, origin recognition complex (ORC) is recruited to initiate and control replication.

In S. cerevisiae, replication origins are defined by consensus DNA sequence elements, called autonomously replicating sequences (ARS) that support efficient DNA replication initiation of extrachromosomal DNA. ARS are about 100-200 base pairs long, and comprises a conserved ARS consensus sequence (ACS). The ARS serves as the primary binding site for the hexameric origin recognition complex (ORC).

In some embodiments, the genetic construct comprises an origin of replication. In some embodiments, the origin of replication is a strong replication origin. In some embodiments, the origin of replication is an early-firing autonomously replicating sequence. In another embodiment, the origin of replication is an ARS. There are many known ARSs, and suitable ARS would be known to the person skilled in the art (see for example, Liachko et al. (2011) BMC Genomics 12:633). In some embodiments, the ARS can be an artificial ARS. In a preferred embodiment, the origin of replication is ARS306 or ARS1max.

### 3.3 Gene Transfer/Introduction

The nucleic acid construct, expression cassette or expression vector according to the present disclosure may be transferred into a cell by any suitable method known to persons skilled in the art, illustrative examples of which include electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation and liposome-mediated transformation.

Transformation allows uptake and incorporation of the exogenous genetic material, to effect stable, heritable alteration in the cell genome. Exogenous nucleotides may include gene foreign to the target organism or addition of a nucleotide sequence present in the wild-type organism. The results of a stable genetic modification caused by transformation is maintained in at least a portion of a population of cells for ten or more generations or for a length of time equal or greater to ten times the average generation time for the modified organism.

### 3.4 Cells

Also provided herein is a cell comprising the nucleic acid construct as described herein.

The cell of the present disclosure is a cell that comprises haploinsufficient genes. The cell may be a prokaryote or a eukaryote or an archaean cell. The prokaryotic cell may be any Gram-positive or Gram-negative bacterium. In some embodiments the bacterial cell is selected from the group of Escherichia coli, Pseudomonas, Bacillus, and Streptomyces. In one embodiment, the bacteria may be Bacillus subtilis. In another embodiment, the bacteria may be Clostridium saccharoperbutylacetonicum. In one embodiment, the cell is a cyanobacteria cell. In some embodiments the cyanobacteria is a Synechocystis spp., Cyanothece spp., Nostoc spp., Scytonema spp., Arthrospira spp. such as Arthrospira platensis, Arthrospira fusiformis and Arthrospira maxima, or Microcystis aeruginosa. The cell may also be a eukaryotic cell, such as a yeast, fungal, algal, microalgal, mammalian, insect or plant cell. In some embodiments, the cell is an algae or a microalgae. In some embodiments, the algae or microalgae is a kelp or seaweed or sea lettuce (Ulva spp.), such as brown algae or Sargassum spp. including Sargassum fusiforme. In some embodiments, the algae or microalgae is Chlorella spp., Dunaliella spp., Gracilaria spp., Eucheuma spp., Saccharina japonica, Gracilaria spp., Pyropia spp., Chlamydomonas spp., Haematococcus spp., Kappaphycus alvarezii or Undaria pinnatifida. In some embodiments the algae or microalgae is Ankistrodesmus spp., Botryococcus braunii, Crypthecodinium cohnii, Cyclotella spp., Hantzschia spp., Nannochloris spp., Nannochloropsis spp., Neochloris oleoabundans, Nitzschia spp., Phaeodactylum tricornutum, Scenedesmus spp., Schizochytrium spp., Stichococcus spp., Tetraselmis suecica or Thalassiosira pseudonana. In a particular embodiment, the cell is a yeast cell. In a further particular embodiment, the yeast cell is selected from the group of Trichoderma, Aspergillus, Saccharomyces, Schizosaccharomyces, Kluyveromyces, Torulaspora, Pichia, Thermus, Hansenula, Torulopsis, Komagataella, Candida, Karwinskia or Yarrowia. In representative embodiments, the yeast is selected from Saccharomyces species (e.g., Saccharomyces cerevisiae), Kluyveromyces species (e.g., Kluyveromyces lactis), Torulaspora species, Yarrowia species (e.g., Yarrowia lipolitica), Schizosaccharomyces species (e.g., Schizosaccharomyces pombe), Pichia species (e.g., Pichia pastoris or Pichia methanolica), Hansenula species (e.g., Hansenula polymorpha), Torulopsis species, Komagataella species, Candida species (e.g., Candida boidinii), and Karwinskia species. In another embodiment, the cell is S. cerevisiae or S. pombe or a Pichia species. The cell may be any cell useful in the production heterologous gene products. The cell may be any cell that is suitable for function as cell factories, which will be known or easily recognised by the person skilled in the art.

In some embodiments, the cell of the present disclosure is a cell that is produced by any of the methods disclosed herein.

The cell may be any cell useful in the production heterologous gene products. The cell may be a prokaryote or a eukaryote. The prokaryotic cell may be any Gram-positive or Gram-negative bacterium. The cell may also be a eukaryotic cell, such as a yeast, fungal, mammalian, insect or plant cell. In particular embodiments, the cell is selected from the group of Escherichia coli, Pseudomonas, Bacillus, Streptomyces, Trichoderma, Aspergillus, Saccharomyces, Pichia, Thermus or Yarrowia. Any cell that is suitable for function as cell factories will be known or easily recognized by the person skilled in the art.

As used herein, the cell has introduced into it exogenous nucleic acids, such as a vector or other polynucleotides. The cell may be transformed, transfected or transduced in a transient or stable manner. The polynucleotide construct, expression cassette or vector is introduced into a host cell so that the polynucleotide, cassette or vector is maintained as a chromosomal integrant or as a self-replicating extra-chromosomal vector.

The cell may comprise one copy of the nucleic acid construct in its genome. The cell of the present disclosure may comprise 2 to 200 copies, suitably 3 to 100 copies, suitably 3 to 70 copies, suitably 3 to 60 copies of the nucleic acid construct. The nucleic acid construct may be amplified to form a transgenic tandem amplified region in the genome of the cell, wherein the transgenic tandem amplified region comprises multiple copies of the nucleic acid construct. In one embodiment, the recombinant cell may comprise of more than one transgenic tandem amplified region in its genome.

In some embodiments, the nucleic acid construct that is amplified in the cell comprises origin of replications, in preferred embodiments, the nucleic acid construct that is amplified in the recombinant yeast cell comprises the autonomous replicating sequences ARS306 or ARS1max.

### 4. Expression of Heterologous Nucleic Acids and/or Proteins

The methods, nucleic acid constructs and cells disclosed herein are useful for increasing expression of introduced genes, transgenes and heterologous proteins in cells, such as in the industrial production of biofuels, proteins, biochemicals, chemicals, enzymes, pharmaceuticals and biopharmaceuticals. Genes and products that can be expressed using the present disclosure can also be used in the synthesis of other products, including phenolics, isoprenoids, alkaloids, and polyketides. Biopharmaceuticals include vaccines, insulin, antibodies, erythropoietin, hormones, blood factors, interferons, interleukins, growth factors, fusion proteins, recombinant enzymes. Other useful products that can be expressed in the cell of the present invention, for example, include flavor and fragrance compositions for use in food, medicine and cosmetic preparations.

Thus provided herein is a method of expressing a nucleic acid in a cell, the method comprising culturing the cell disclosed herein or a cell produced by any one of the methods disclosed herein, to express the nucleic acid construct comprising the corresponding nucleic acid.

The cell comprising the nucleic acid construct of the present disclosure may be cultivated in a nutrient medium suitable for production of the gene product (i.e. a polypeptide or nucleic acid) encoded by the heterologous nucleic acid. The cell can be cultivated or cultured for a period of time and/or under the appropriate conditions to allow expression of the gene product or synthesis of a related product, using methods that will be known to persons skilled in the art. Suitable examples include cultivating the cell by shake flask cultivation, or small-scale or large-scale fermentation (including continuous, batch, fed-batch, or solid state fermentations) in laboratory or industrial fermenters performed in a suitable medium and under conditions allowing the gene product/product to be expressed and/or isolated. The cultivation will typically take place in a suitable nutrient medium, from commercial suppliers or prepared according to published compositions or any other culture medium suitable for cell growth.

Where the expressed gene product or related product is secreted into the nutrient medium, it can be recovered directly from the culture supernatant. Optionally, the gene product or related product can be recovered or purified from cell lysates or after permeabilization of the host cell membrane. The gene product or product may be recovered purified using any suitable method known to persons skilled in the art, illustrative examples of which include collection, centrifugation, filtration, extraction, spray-drying, evaporation, or precipitation. Optionally, the gene product or related product may be partially or totally purified by a variety of procedures known in the art including, but not limited to, thermal shock, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction to obtain substantially pure fractions of the gene product or related product.

The gene product or related product may be used, in crude or purified form, either alone or in combination with additional products. The present disclosure also extends to compositions comprising the gene product or related product, the nucleic acid construct or the cell described herein.

The composition may be liquid or dry, for instance in the form of a powder. In some embodiments, the composition is a lyophilizate. For instance, the composition may comprise the gene product, nucleic acid construct and/or cells and optionally excipients and/or reagents etc. Suitable excipients may include buffers commonly used in biochemistry, agents for adjusting pH, preservatives such as sodium benzoate, sodium sorbate or sodium ascorbate, conservatives, protective or stabilizing agents such as starch, dextrin, arabic gum, salts, sugars e.g., sorbitol, trehalose or lactose, glycerol, polyethyleneglycol, polyethene glycol, polypropylene glycol, propylene glycol, divalent ions such as calcium, sequestering agent such as EDTA, reducing agents (e.g., beta-mercaptoethanol, dithiothreitol, ascorbic acid, tris(2-carboxyethyl)phosphine), amino acids, a carrier such as a solvent or an aqueous solution, and the like. The excipient may be polyvinylalcohol (PVA) and co-polymers thereof with PVP or with other polymers, polyacrylates, urea, chitosan and chitosan glutamate, sorbitol or other polyols such as mannitol. The excipient may be PVPK30, cellulose derivatives, such as, but not limited to, polyvinylpyrrolidone, polyethylene-/polypropylene-/polyethylene-oxide block copolymers such as Pluronic F68, polymethacrylates, sodium dodecyl sulfate, polyoxyethylene sorbitan fatty acid esters such as Tween 80, bile salts such as sodium deoxycholate, polyoxyethylene mono esters of a saturated fatty acid such as Solutol HS 15, water soluble tocopheryl polyethylene glycol succinic acid esters such as Vitamin E TPGS, hydroxypropylcellulose (HPC), hydroxypropylmethylcellulose (HPMC), hydroxypropylmethylcellulose acetate succinate (HPMC-AS), hydroxypropylcellulose phthalate (HPMC-P), methylcellulose (MC), polyethyleneglycols, and earth alkali metal silicas and silicates, e.g. fumed silicas, precipitated silicas, calcium silicates, such as Zeopharm®600, or magnesium aluminometasilicates such as Neusilin US2. The gene product as described herein is solubilized together with one or more excipients, such as excipients that may suitably stabilize or protect the gene product from degradation.

The excipients may function as a carrier or a diluent to preserve or alter a particular quality of the composition such as the effectiveness, stability, dispersiveness, miscibility wettability, texture, taste or aroma. The excipient may be a bulking agent, or an anti-fouling agent, or an anti-caking agent. Examples of appropriate excipients include, but not limited to bonding agents (for example, microcrystalline cellulose, tragacanth or bright Glue), coatings, disintegrants, fillers, diluents, softening agents, sweeteners, emulsifying agents, natural flavoring, artificial flavor enhancements (e.g. NaCl, KCl, MSG, guanosine monophosphate (GMP), inosin monophospahte (IMP), ribonucleotides such as disodium inosinate, disodium guanylate, N-(2-hydroxyethyl)-lactamide, N-lactoyl-GMP, N-lactoyl tyramine, gamma amino butyric acid, allyl cysteine, 1-(2-hydroxy-4-methoxylphenyl)-3-(pyridine-2-yl) propan-1-one, arginine, potassium chloride, ammonium chloride, succinic acid, N-(2-methoxy-4-methyl benzyl)-N′-(2-(pyridin-2-yl)ethyl)oxalamide, N-(heptan-4-yl)benzo(D)(1,3)dioxole-5-carboxamide, N-(2,4-dimethoxybenzyl)-N′-(2-(pyridin-2-yl)ethyl)oxalamide, N-(2-methoxy-4-methyl benzyl)-N′-2(2-(5-methyl pyridin-2-yl)ethyl)oxalamide, cyclopropyl-E,Z-2,6-nonadienamide), colouring agents, lubricants, functional agent (for example, nutrients), viscosity modifiers, fillers, glidants (for example, cataloid), surfactants or infiltration agents. Other examples of excipients include silicon dioxide (silica, silica gel), carbohydrates and/or carbohydrate polymers (polysaccharides), cyclodextrins, starches, degraded starches (starch hydrolysates), chemically or physically modified starches, modified celluloses, pectin, inulin, maltodextrins and dextrins. The excipient may be a acetin, magnesium stearate, hydrogenated vegetable oil, essential oil, plant extracts, fruit essence, spices, extracts, oils, gelatin, alcohols, triacetine, glycerol, miglycol, acetaldehyde, dimethyl sulfide, ethyl acetate, ethyl propionate, methyl butyrate, and ethyl butyrate.

The carrier or excipient may function as a processing aid or to shield or protect the other components from the effects of moisture, light, or oxygen or any other aggressive media. The carrier material might also act as a means of controlling the release of flavor or aroma from the composition, or control the degradation or release of the active compound. Further examples of carriers and excipients include sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum acacia, tragacanth, alginates, methyl cellulose, carboxymethyl cellulose, hydroxyethyl cellulose, hydroxypropylmethyl cellulose, derivatives and mixtures thereof.

Suitable excipients would depend on the composition and its intended use, therefore selection of the appropriate excipient would be known to the skilled person. The skilled person will appreciate that the cited materials are hereby given by way of example and are not to be interpreted as limiting the invention.

It will be appreciated that the above described terms and associated definitions are used for the purpose of explanation only and are not intended to be limiting.

In order that the disclosure may be readily understood and put into practical effect, particular preferred embodiments will now be described by way of the following non-limiting example.

### Representative Embodiments of the Disclosure

1. A method for increasing copy number of a haploinsufficient gene in the genome of a cell, the method comprising, consisting or consisting essentially of reducing expression of the haploinsufficient gene to thereby increase the copy number of the haploinsufficient gene in the genome of the cell.

2. The method of embodiment 1, wherein the haploinsufficient gene is operably connected to an origin of replication.

3. A method for increasing copy number of a heterologous nucleic acid sequence in the genome of a cell, the method comprising, consisting or consisting essentially of: introducing the heterologous nucleic acid sequence into the genome, wherein the heterologous nucleic acid sequence is introduced in operable connection with a haploinsufficient gene of the genome; and reducing expression of the haploinsufficient gene, wherein the reduced expression of the haploinsufficient gene increases copy number in the genome of a nucleic acid construct comprising the heterologous nucleic acid sequence and the haploinsufficient gene, thereby increasing the copy number of the heterologous nucleic acid sequence in the genome of the cell.

4. The method of embodiment 3, wherein the heterologous nucleic sequence comprises at least one coding sequence in operable connection with a promoter that is operable in the cell.

5. The method of embodiment 3 or embodiment 4, wherein the heterologous nucleic sequence is located upstream or downstream of the haploinsufficient gene.

6. The method of any one of embodiments 1 to 5, wherein the nucleic acid construct comprises an origin of replication.

7. The method of any one of embodiments 1 to 6, wherein the method excludes rescuing expression of the haploinsufficient gene through use of a separate rescuing agent.

8. The method of any one of embodiments 1 to 7, wherein expression of the haploinsufficient gene is reduced by any one or more of the following:
a. replacing the endogenous promoter of the haploinsufficient gene with a weaker promoter;
b. replacing or adding at least one codon of the haploinsufficient gene with a codon that has a lower translational efficiency in the cell;
c. disrupting the haploinsufficient gene;
d. modifying the haploinsufficient gene to include a nucleotide sequence encoding an RNA destabilizing element; and
e. expressing a nucleic acid molecule in the cell, which reduces the level of an expression product of the haploinsufficient gene.

9. The method of any one of embodiments 1 to 8, wherein the increased copy number of the haploinsufficient gene or the heterologous nucleic acid sequence is from 2 to 200 copies, suitably 3 to 100 copies, suitably 3 to 70 copies, suitably 3 to 60 copies.

10. The method of any one of embodiments 1 to 9, wherein the cell is a yeast, fungal, bacterial, algal, microalgae, cyanobacterial, insect or mammalian cell, suitably a yeast cell.

11. The method of any one of embodiments 1 to 10, wherein the haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11.

12. The method of any one of embodiments 1 to 11, wherein expression of the haploinsufficient gene is reduced by replacing the endogenous promoter of the haploinsufficient gene with a weaker promoter, wherein the weaker promoter is selected from the group consisting of ERG1 promoter, PDA1 promoter, BTS1 promoter, GLO2 promoter and COG7 promoter.

13. The method of any one of embodiments 1 to 12, wherein the haploinsufficient gene is operably connected to an origin of replication, wherein the origin of replication is ARS306 or ARS1max.

14. A cell that is produced by any one of the methods of embodiments 1 to 13.

15. A nucleic acid construct comprising a recombinant polynucleotide that reduces expression of a haploinsufficient gene that is endogenous to a cell of interest.

16. The nucleic acid construct of embodiment 15, further comprising a heterologous nucleic acid sequence in operable connection with the haploinsufficient gene.

17. The nucleic acid construct of embodiment 16, wherein the heterologous nucleic sequence comprises at least one coding sequence in operable connection with a promoter that is operable in the cell.

18. The nucleic acid construct of embodiment 16 or embodiment 17, wherein the heterologous nucleic sequence is located upstream or downstream of the recombinant polynucleotide.

19. The nucleic acid construct of any one of embodiments 15 to 18, further comprising an origin of replication.

20. The nucleic acid construct of any one of embodiments 15 to 19, wherein the recombinant polynucleotide is selected from:
a. a polynucleotide that comprises a promoter that is weaker than the endogenous promoter of the endogenous haploinsufficient gene, which when introduced into the genome of the cell, is operably connected to the haploinsufficient gene;
b. a modified haploinsufficient gene that is distinguished from the endogenous haploinsufficient gene by replacement of the endogenous promoter of the endogenous haploinsufficient gene with a weaker promoter, and/or replacement or addition of at least one codon of the endogenous haploinsufficient gene with a codon that has a lower translational efficiency in the cell;
c. a modified haploinsufficient gene that is distinguished from the endogenous haploinsufficient gene by disruption of endogenous haploinsufficient gene;
d. a modified haploinsufficient gene that is distinguished from the endogenous haploinsufficient gene by operably connecting a nucleotide sequence encoding an RNA destabilizing element to the endogenous haploinsufficient gene; and
e. a polynucleotide that reduces the level of an expression product of the haploinsufficient gene.

21. The nucleic acid construct of any one of embodiments 15 to 20, wherein the recombinant polynucleotide is distinguished from the endogenous haploinsufficient gene by replacement of the endogenous promoter of the endogenous haploinsufficient gene with a weaker promoter, wherein the weaker promoter is selected from the group consisting of ERG1 promoter, PDA1 promoter, BTS1 promoter, GLO2 promoter and COG7 promoter.

22. The nucleic acid construct of any one of embodiments 15 to 21, wherein the haploinsufficient gene is a gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11.

23. The nucleic acid construct of any one of embodiments 19 to 22, wherein the origin of replication is an autonomous replicating sequence, where in the autonomous replicating sequence is ARS306 or ARS1max.

24. The nucleic acid construct of any one of embodiments 17 to 23, wherein the coding sequence encodes an expression product selected from a polypeptide, (e.g. a polypeptide for producing a terpenoid, a flavonoid or a fatty acid, an antibody, a nanobody) or a functional RNA molecule (e.g., RNAi that inhibits expression of a target gene).

25. A cell comprising the nucleic acid construct of any one of claims 15 to 24.

26. The cell of embodiment 25, wherein the cell comprises 2 to 200 copies, suitably 3 to 100 copies, suitably 3 to 70 copies, suitably 3 to 60 copies.

27. The cell of embodiment 25 or embodiment 26, wherein the cell is a yeast, bacterial, archaean, algal, microalgae, cyanobacterial, insect or mammalian cell, suitably a yeast cell.

28. A method for expressing nucleic acid, the method comprising:
culturing the cell of any one of embodiments 25 to 27 to express the nucleic acid construct of any one of embodiments 15 to 24.

29. The cell of any one of embodiments 25 to 27, wherein the nucleic acid construct comprises the haploinsufficient gene ribosomal 60S subunit protein L25, wherein the haploinsufficient gene ribosomal 60S subunit protein L25 is operably connected to a weaker promoter that is weaker that the native ribosomal 60S subunit protein L25, wherein the weaker promoter is selected from ERG1 promoter, PDA1 promoter, BTS1 promoter, GLO2 promoter and COG7 promoter.

30. The cell of embodiment 29, wherein the haploinsufficient gene ribosomal 60S subunit protein L25 is operably connected to the ERG1 promoter.

31. The cell of embodiment 29, wherein the haploinsufficient gene ribosomal 60S subunit protein L25 is operably connected to the PDA1 promoter.

32. The cell of embodiment 29, wherein the haploinsufficient gene ribosomal 60S subunit protein L25 is operably connected to the BTS1 promoter.

33. The cell of any one of embodiments 25 to 27, wherein the nucleic acid construct comprises the haploinsufficient gene GTPase-activating protein SEC23, wherein the haploinsufficient gene GTPase-activating protein SEC23 is operably connected to a weaker promoter that is weaker that the native GTPase-activating protein SEC23, wherein the weaker promoter is selected from ERG1 promoter, PDA1 promoter, BTS1 promoter, GLO2 promoter and COG7 promoter.

34. The cell of embodiment 33, wherein the haploinsufficient gene GTPase-activating protein SEC23 is operably connected to the ERG1 promoter.

35. The cell of embodiment 33, wherein the haploinsufficient gene ribosomal 60S subunit protein L25 is operably connected to the PDA1 promoter.

36. The cell of embodiment 33, wherein the haploinsufficient gene ribosomal 60S subunit protein L25 is operably connected to the BTS1 promoter.

37. The cell of embodiment 33, wherein the haploinsufficient gene ribosomal 60S subunit protein L25 is operably connected to the GLO2 promoter.

38. The cell of embodiment 33, wherein the haploinsufficient gene ribosomal 60S subunit protein L25 is operably connected to the COG7 promoter.

39. The cell of any one of embodiments 25 to 38, wherein the haploinsufficient gene comprises at least one codon that has a lower translational efficiency.

### EXAMPLES

### Example 1

### Materials and Methods

### Construct Design for In Vivo Gene Amplification (HapAmp)

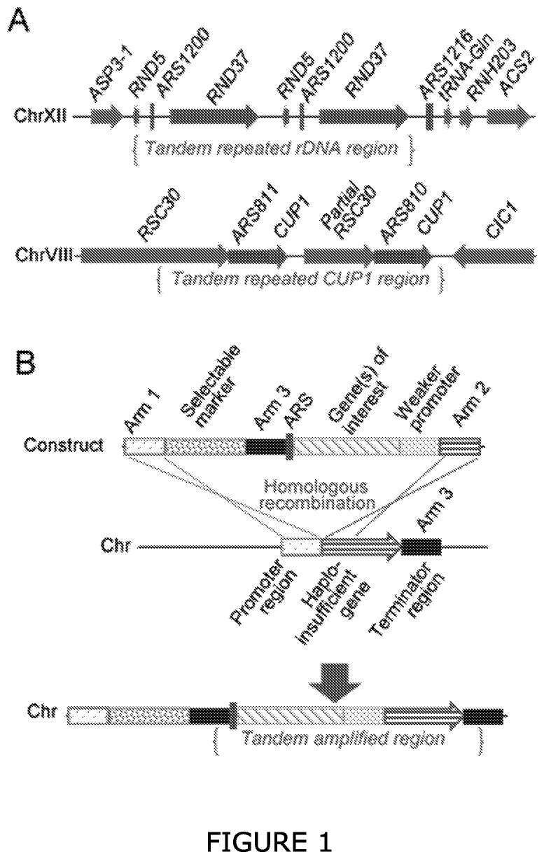

The likelihood of gene amplification is increased when there is: (1) a gene linked to cell fitness, and (2) homologous DNA sequences to support recombination. In addition, a strong replication origin can promote amplification. These three elements exist in tandem repeat in the rDNA region and the CUP1 region in the yeast genome (FIG. 1a).

A genetic construct was designed to enable gene amplification in yeast (FIG. 1b). The construct has recombination arms or homologous arms. In this example, Arm 1 is homologous to the promoter region of a haploinsufficient gene, and Arm 2 is homologous to the initial part of open reading frame of the haploinsufficient gene. This allows insertion of the construct onto the genome by homologous recombination. Downstream of Arm 1 resides a selectable marker for transformation selection and homologous Arm 3, which is homologous to the terminator region of the haploinsufficient gene. Between Arm 3 and Arm 2, there are an autonomous replicating sequence (ARS; the yeast origin of replication), and a promoter.

The promoter element of the genetic construct is weaker than the native promoter of the haploinsufficient gene and positioned such that integration results in substitution of the native promoter of the haploinsufficient gene with the weaker promoter. Genes of interest or transgenes to be amplified and/or expressed heterologously, can be inserted between Arm 3 and the weaker promoter.

Driving expression through a weaker promoter attenuates the protein yield from haploinsufficient gene immediately downstream of the promoter. This, in turn, is expected to decrease the cell fitness in yeast. Native amplification of the region between homologous Arm 3 in the construct and Arm 2 (or Arm3 naturally existing in genome) will then occur as yeast evolves to recover fitness.

### Plasmid and Strain Construction

Plasmids used in this work are listed in Table 2, and strains are listed in Table 3. Primers used in polymerase chain reaction (PCR) and PCR performed in this work are listed in Table 4. Plasmid construction processes are listed in Table 5. Yeast strain construction processes are listed in Table 6. A LiAc/SS carrier DNA/PEG method (Gietz, R. D. & Schiestl, Nature Protocols 2, 38-41 (2007)) was used for yeast transformation.

### Yeast Cultivation

For characterization of yEGFP-expressing strains, yeast cells from glycerol stocks were streaked on YNB-glucose agar, which comprised of 6.9 g L⁻¹ yeast nitrogen base without amino acids (YNB, FORMEDIUM #CYN0402) with pH adjusted to 6.0 using sodium hydroxide solution, 20 g L⁻¹ glucose, and 20 g L⁻¹ agar. MES-buffered YNB-glucose medium was used in following cultivation, which comprised of 19.5 g L⁻¹ 2-(N-morpholino) ethanesulfonic acid (MES), 6.9 g L⁻¹ YNB, 20 g L⁻¹ glucose, and its pH was adjusted to 6.0 with ammonia hydroxide solution. For the growth in flask, seed cultures grown to the exponential phase (OD600≤4) were inoculated into 20 ml MES-buffered YNB-glucose medium in 125 ml Erlenmeyer flasks to start the cultivation in a 200 rpm 30° C. incubator. For the growth in 96-well microplate, yeast cells were grown in YNB-glucose medium (6.9 g L⁻¹ YNB, 20 g L⁻¹ glucose, pH 6.0) for about 20 hour to stationary phase in a 350 rpm 30° C. incubator to prepare seed culture. Seed culture (5 μl) was inoculated into 100 μl MES-buffered YNB-glucose medium to prepare Culture 1. Culture 1 (2 μl) was inoculated into 100 μl MES-buffered YNB-glucose medium to prepare Culture 2. Culture 2 was incubated in a 350 rpm 30° C. incubator overnight for analysis of yEGFP fluorescent in the cells grown to the exponential growth phase, and Culture 1 for two nights for analysis in the cells grown to the ethanol growth phase.

For characterization of nerolidol/limonene-producing strains, dodecane-overlayed two-phase flask cultivation was used. Yeast cells from glycerol stocks were streaked on YNB-high-glucose agar, which contained 6.9 g L⁻¹ YNB (pH 6.0), 200 g L⁻¹ glucose, and 20 g L⁻¹ agar. Before initiating the two-phase flask cultivation, cells were pre-cultured in MES-buffered YNB-20 g L⁻¹ glucose to exponential phase (OD600 between 1 to 4) and collected by centrifugation. Collected cells were then resuspended in fresh fermentation medium. To initiate the cultivation, appropriate volumes of pre-cultured cells were transferred to MES-buffered YNB medium with 20 g L⁻¹ glucose to an initial OD600 of 0.2 in a total volume of 23 mL medium in a 250 ml flask, and 2 mL sterile dodecane was added after inoculation. In the first 12 hours of cultivation, 3 ml culture was sampled for growth curve measurement. Dodecane was sampled and stored at −80° C. for terpene analysis.

Flask cultivations for lycopene-producing strains were prepared as the flask cultivation used for yEGFP-expressing strains.

For chromoprotein/HPV-expressing strains, yeast cells grown overnight in 5 ml MES-buffered YNB-glucose medium were inoculated into 20 ml fresh MES-buffered YNB-glucose medium or 20 ml YP-galactose (20 g L⁻¹ peptone, 10 g L⁻¹ yeast extract, and 20 g L⁻¹ galactose) to start characterization cultures.

### Flow Cytometry

Fluorescence in single cells was analyzed using a BD Accuri™ C6 flow cytometer (BD Biosciences, USA). For analysis of yEGFP fluorescence, cells sampled from characterizations were directly used for flow cytometry analysis. For analysis of Y-FAST fluorescence, 100-time-concentrated HMBR, synthesized as reported previously and dissolved in dimethyl sulfoxide, was added to the samples to 20 μM final concentration and the sample was mixed before analysis. FSC.H threshold was set at the value of 250,000 for exclusion of debris particles. GFP and/or Y-FAST fluorescence was excited by a 488 nm laser and monitored through a 530/20 nm bandpass filter (FL1.A), with 10,000 events recorded per sample. Mean values of FSC.A, SSC.A, and FL1.A for all detected events were extracted using a BD Csampler software (BD Accuri C6 software version 1.0.264.21). GFP or Y-FAST fluorescence level was expressed as the percentage of the average background auto-fluorescence from the exponential-phase cells of GFP-negative reference strain GH4 as described previously.

### Metabolite Analysis

The Metabolomics Australia Queensland Node analyzed extracellular metabolites. Sesquiterpenes and monoterpenes in dodecane samples were analyzed as previously described (Peng, B. et al. Metabolic engineering 39, 209-219 (2017)). Dodecane samples (in some cases, diluted with dodecane) were diluted in 40-fold volume of ethanol. The ethanol-diluted samples (20 μL) were injected. A Zorbax Extend C18 column (4.6×150 mm, 3.5 μm, Agilent PN: 763953-902) equipped with a guard column (SecurityGuard Gemini C18, Phenomenex PN: AJO-7597) was used. Analytes were eluted at 35° C. at 0.9 mL/min using the mixture of solvent A (water) and solvent B (45% acetonitrile, 45% methanol, and 10% water), with a linear gradient of 5-100% solvent B from 0-24 min, then 100% from 24-30 min, and finally 5% from 30.1-35 min. Analytes of interest were monitored using a diode array detector (Agilent DAD SL, G1315C) at 202 nm wavelength. Analytical standards were used to prepare the standard curve for quantification.

For lycopene measurement, yeast cells were collected and resuspended in 200 μL 2 M L⁻¹ sodium hydroxide and vortexed with 200 mg glass bead and 1 mL hexane for at least 10 min. Lycopene concentration was calculated from the absorbance of hexane extracts at 471 nm. Dilution was performed to make absorbance reading <0.6. Lycopene molar extinction coefficient (182×10³) was used to calculate lycopene concentration (Takehara, M. et al. Journal of agricultural and food chemistry 62, 264-269 (2014)).

### Protein Purification

Yeast cells were homogenized by vortexing with glass beads for 15 min in phosphate-buffered saline (PBS) buffer plus 2 mM ethylenediaminetetraacetic acid (EDTA). Whole-cell lysates, lysate supernatants, and lysate pellets were examined by sodium dodecyl sulfate-polyacrylamide gel electrophoresis analysis on Mini-PROTEAN® Precast Gels (Bio-rad).

The lysis was followed by centrifugation at 18000×g for 30 minutes to pellet the cellular debris. The soluble fraction was then loaded on top of a gradient made of 1 mL of 20% Iodixanol/PBS buffer, 1 mL of 30% Iodixanol/PBS and 1 mL of 40% Iodixanol/PBS in a Thinwall Ultra-Clear Tube (Beckman Coulter, Indianapolis, USA) and subjected to ultracentrifugation for 2 hours 30 minutes at 150,000 g on a SW41 Ti rotor or a using a Beckman Optima L-100XP ultracentrifuge (Beckman Coulter, Indianapolis, USA). A band containing the virus-like particles encapsulating protein was extracted using a 1 ml syringe by poking a whole through the tube. Bradford was used to measure protein concentration and sample was further examined on TEM and purity confirmed on Mini-PROTEAN® Precast Gels (Bio-Rad).

### Transmission Electron Microscopy

Samples containing purified VLPs of 0.1 mg mL-1 were applied to formvar/carbon coated grids (ProSciTech Pty Ltd, Australia) and incubated for 2 minutes. Grids were then washed with 40 μL of distilled water for 30 sec twice, and then stained with 20 g L⁻¹ uranyl acetate for 1 minute, after being blotted on filter paper. Images were taken on a HITACHI HT7700 transmission electron microscope at accelerating voltage of 80 keV at the Centre for Microscopy and Microanalysis.

### Genome Sequencing

Yeast genomic DNA was extracted using MagAttract HMW DNA Kit (Qiangen) with a modified protocol. Yeast cells (20 ml, OD₆₀₀ around 10) were washed once using phosphate-buffered saline (PBS) buffer and resuspend in 2 ml 1M sorbitol solution. Yeast cell walls were digested by adding 30 U Zymolyase-20T (nacalai, Japan; 1 U per μl in 1*PBS containing 100 mM DTT and 50% v/v glycerol) at 30° C. for 30 minutes. Yeast protoplast cells were collected and resuspended in 300 μl Buffer AL (MagAttract HMW DNA Kit) by pipetting using wide bore pipette tips, and then 360 buffer ATL (MagAttract HMW DNA Kit) was added and mixed. Following this, protocol provided in MagAttract HMW DNA Kit (Qiangen) was adopted including digestion by Proteinase K and Rnase A and purification using magnetic beads. Genomic DNA was eluted using 400 μl Buffer AE (MagAttract HMW DNA Kit) and treated using 100 μl tris-saturated phenol (pH 8.0, Ameresco) by flickering and 100 μl chloroform was added and mixed. Upper-layer water phase was collected after centrifuging at 17,000 g for 5 minutes and mixed with 1 ml ethanol. Magnetic beads (MagAttract HMW DNA Kit) was used to purify genomic DNA with twice 70% ethanol wash and elution in 50 μl water. Concentration of genomic DNA was quantified using Qubit Fluorometer and Qubit dsDNA BR Assay Kit (Thermo Fisher). Genomic DNA (500 ng) was used to prepare genome sequencing library using Rapid Barcoding Kit (SQK-RBK004, Oxford Nanopore) and sequenced using R9 flowcell MIN106D and MinION Mk1C (Oxford Nanopore). High-accurate basecalling was performed using Guppy ( ) installed MinION Mk1C. Galaxy Australia online server was used for data processing. Collapse Collection (Galaxy Version 5.1.0) was used to combine fastq dataset into a single file. Nanoplot was used for statistical analysis of MinION reads. Canu assembler was used for genome sequence assembly. Maker (Galaxy Version 2.31.11) was used to collect annotation evidence with input of S. cerevisiae gene sequences and heterologous gene sequences as ESTs input file. miniMap2 was used to align trimmed reads outputted by Canu assembler against contigs outputted Canu assembler. JBrowse (version 1.16.10-desktop) and Integrative Genomics Viewer (version 2.8.13) were used to illustrate genome structure and read alignment.

### Example 2

### Using RPL25 or SEC23 Haploinsufficient Gene Loci and Promoter Substitution to Drive Gene Amplification

Ribosomal 60S subunit protein L25 (RPL25) and the SEC23-encoding component of the Sec23p-Sec24p heterodimer of the COPII vesicle coat are two haploinsufficient genes shown to have an effect on growth fitness (Deutschbauer et al. (2005) Genetics, 169, 1915-1925). These two genes have the strongest fitness effect in rich medium and in minimal mineral medium.

Four constructs were designed with RPL25 as the haploinsufficient gene that acts as the driving gene (i.e. gene that drives amplification), LEU2 as selection marker, and an early-firing autonomously replicating sequence (ARS) ARS306; and three constructs with SEC23 as the driving gene, hygromycin B resistant gene hphMX as selection marker, and the strong ARS1max ARS.

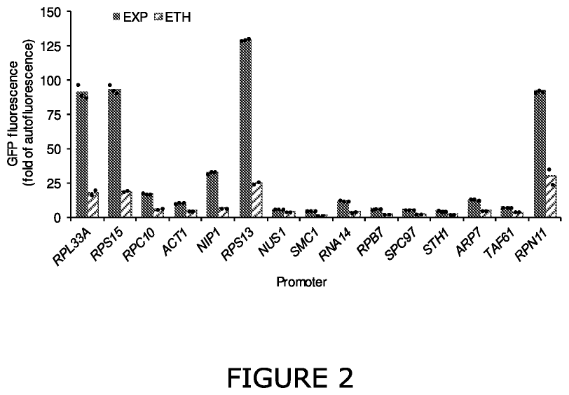

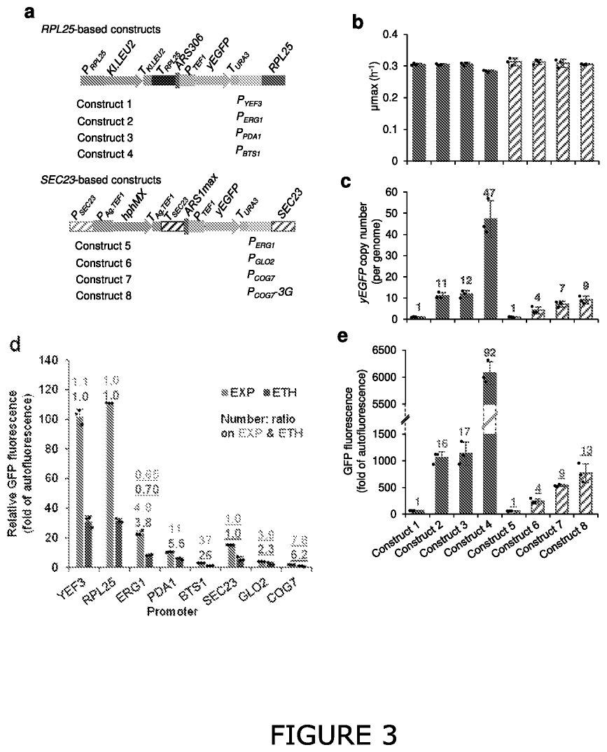

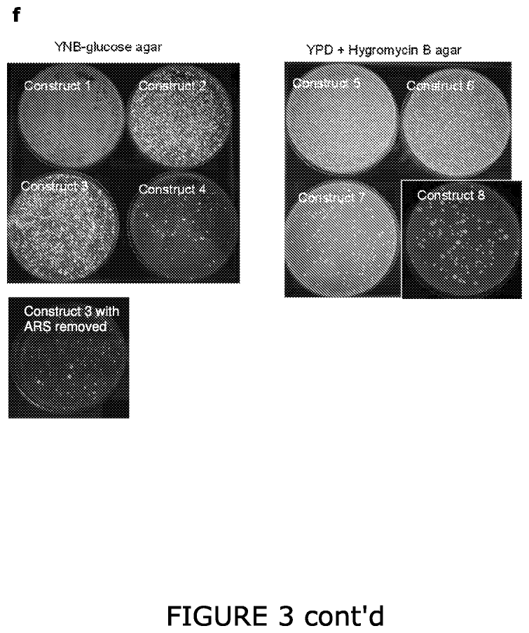

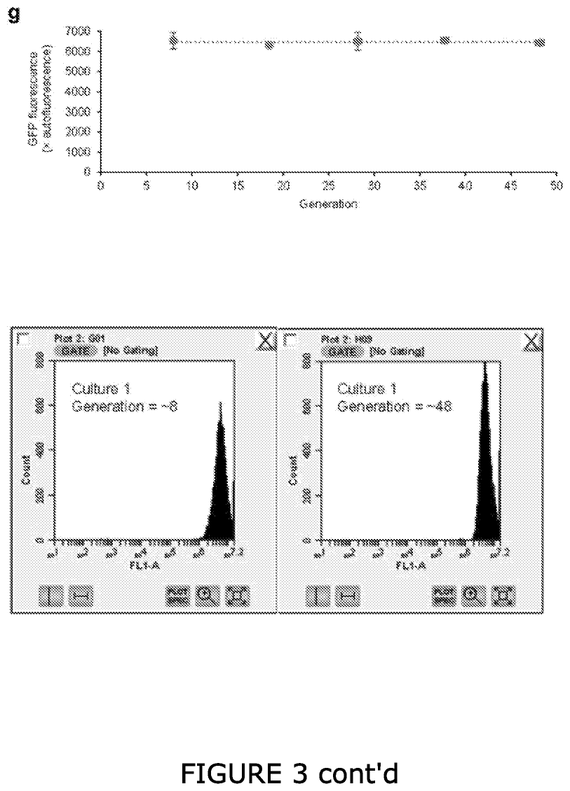

To identify promoters with suitable expression strengths, a wide variety of yeast promoters were tested (see Table 1 below, and FIG. 2) and a sub-set of promoters was selected to test with each target locus (FIGS. 3a & 3d).

TABLE 1
Yeast Promoters

| Promoter | Linked gene |
|---|---|
| RPL33A | 60S ribosomal protein L33-A |
| RPS15 | 40S ribosomal protein S15 |
| RPC10 | DNA-directed RNA polymerases I, II, and III subunit |
| | RPABC4 |
| ACT1 | Actin |
| NIP1 | Eukaryotic translation initiation factor 3 subunit C |
| RPS13 | 40S ribosomal protein S13 |
| NUS1 | Dehydrodolichyl diphosphate synthase complex subunit NUS1 |
| SMC1 | Structural maintenance of chromosomes protein 1 |
| RNA14 | mRNA 3′-end-processing protein RNA14 |
| RPB7 | DNA-directed RNA polymerase II subunit RPB7 |
| SPC97 | Spindle pole body component SPC97 |
| STH1 | Nuclear protein STH1/NPS1 |
| ARP7 | Actin-related protein 7 |
| TAF61 | Transcription initiation factor TFIID subunit 12 |
| RPN11 | Ubiquitin carboxyl-terminal hydrolase RPN11 |

For the RPL25 constructs we used the YEF3 promoter (which has similar strength to the RPL25 promoter; Construct 1 in FIG. 3a) and the ERG1, PDA1, or BTS1 promoters (all with multiple-fold weaker expression than RPL25 promoter; Constructs 2-4 in FIG. 3a). For the SEC23 constructs, we used the ERG1 promoter (stronger than the SEC23 promoter; Construct 5 in FIG. 3a), the GLO2 promoter, or the COG7 promoter (both multiple-fold weaker than the SEC23 promoter; Constructs 6 and 7 in FIG. 3a). An eighth promoter construct was designed using non-preferred codons and tested later (see below). A version of construct 3, without the ARS was also generated. Yeast-enhanced green fluorescent protein (yEGFP) under the control of the TEF1 promoter and the URA3 terminator was used as the gene of interest and as a reporter for proof of concept.

The constructs were transformed into the S. cerevisiae CEN.PK strain. Transformation plates were screened by imaging yEGFP fluorescence under blue light, with imaging of the transformation plates showed fluorescing clones for the 8 constructs tested. Construct 3 without the ARS also lead to the formation of very fluorescent colonies after transformation (FIG. 3f). For each construct 1-8, six strongly-fluorescing clones were selected. Visual observation after sub-culturing demonstrated an inverse correlation between promoter strength (FIG. 3d) and GFP fluorescence. Three clones were selected for further characterization for each construct.

Where promoter strength was similar or greater than the native promoter, yEGFP was found at a single copy on the genome (FIG. 3c: construct 1 & construct 5), and fluorescence (FIG. 3e: construct 1 & construct 5) was similar to fluorescence we observed previously in strains with a single copy of the P_{TEF1}-YEGFP-T_{URA3} construct (Peng, et al. Microbial cell factories 14, 91 (2015)).

However, where the native promoter was substituted for weaker promoters, yEGFP gene copy number and fluorescence both increased (FIGS. 3c & 3e: construct 2-4, 6, 7). Copy number increased from 4-fold to 47-fold, whereas fluorescence increase was 4-fold to 92-fold. There was a strong positive correlation between copy number and fluorescence (r²=0.985), and a weak negative correlation between fluorescence and promoter strength/copy number (r²=0.376 and 0.694 respectively).

The most remarkable result was where the RPL25 promoter was substituted for the BTS1 promoter; this resulted in ˜47 copies of yEGFP per genome and a ˜92-fold increase yEGFP fluorescence (FIGS. 3c & 3e).

The stability of the expression of the yEGFP gene can be maintained long term. The strain comprising construct 4 was cultured for at least 48 generations, to measure the GFP fluorescence levels in the cells over time. For each transferring subculture, cells was inoculated in Yeast extract-Peptone-Glucose (YPD) medium to OD600 equaling to 0.004, grown overnight to OD600˜1 for flow cytometry analysis, and further grown to 24 h to start the next subculture. GFP fluorescence analyses and population homogeneity also did not show significant changes over time (up to at least 48 generations).

### Example 3

### Translational Downregulation Using Non-Preferred Codons to Drive Gene Amplification

To further increase copy number at the SEC23 locus, we attenuated translation by making a construct with three non-preferred glycine codons (GGA) inserted following the start codon of SEC23 under the control of the COG7 promoter (FIG. 3a: Construct 8), which delivered the most gene amplification in the first round (7 copies).

A further increase in gene copy and fluorescence was obtained (FIGS. 3c & 3e). Translational downregulation by use of non-preferred codons provides a second mechanism to drive an increase in copy number for genes at haploinsufficient gene loci.

### Example 4

Growth Rates of Clones with Increased Copy Number

Increased copy number did not negatively impact the growth rate of any of the strains with the exception of clones with the PBTS1-PL25 construct (FIG. 3b), which had a much higher integration copy number than the other clones (FIG. 3c). This strain showed a ˜7% decrease in growth rate (two-tailed t-test p=0.001).

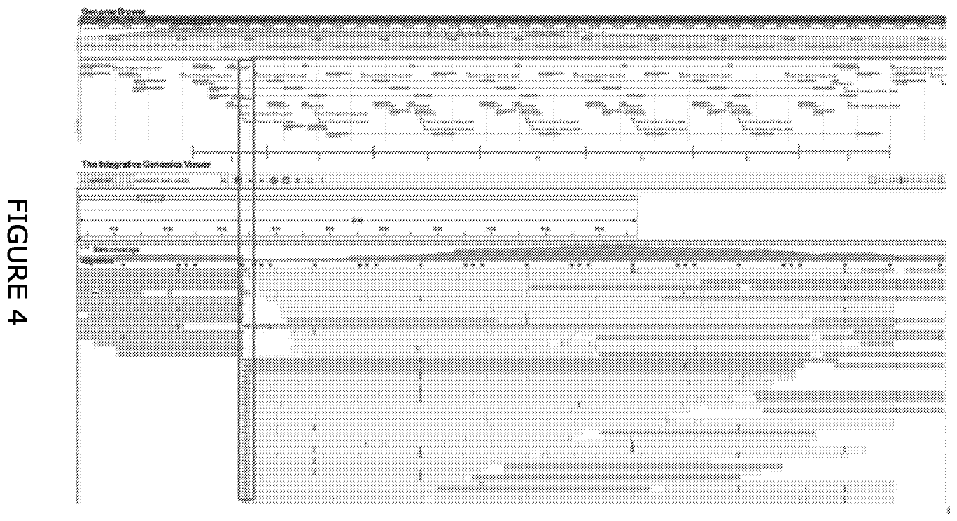

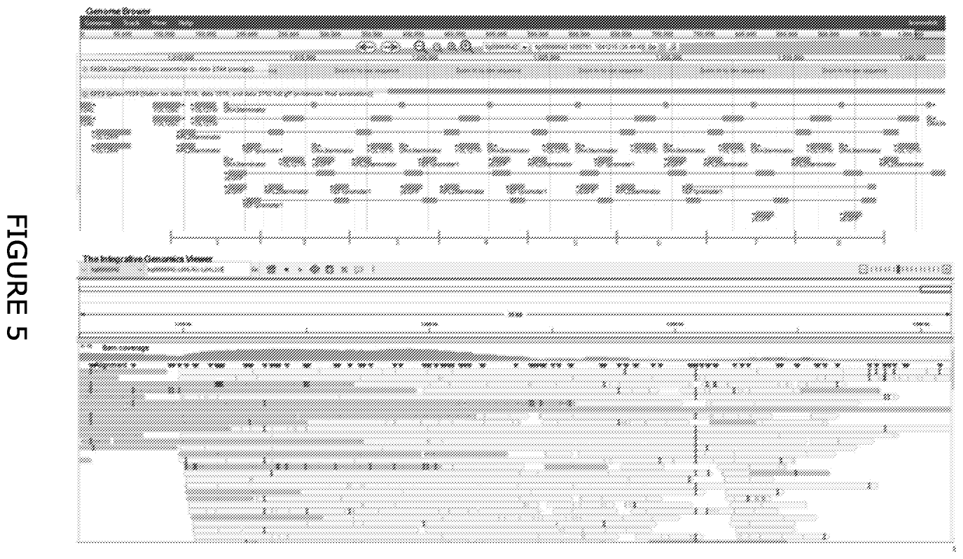

Long-read sequencing on strains containing Construct 3 and Construct 4 confirmed that the constructs were integrated into the RPL25 (YOL127W) locus and that yEGFP-RPL25 sequences were amplified in tandem repeat structures (FIGS. 4 and 5).

### Example 5

### Improving Heterologous Production of the Sesquiterpene Trans-Nerolidol

The performance of the presently described genetic amplification strategy/method for C₁₅ sesquiterpene (trans-nerolidol) production was assessed. A background strain with upregulated mevalonate pathway for production of terpene precursors was used for these experiments. In this strain, the GAL80 repressor gene is disrupted allowing diauxic induction of GAL promoters, which are used to control transgene expression.

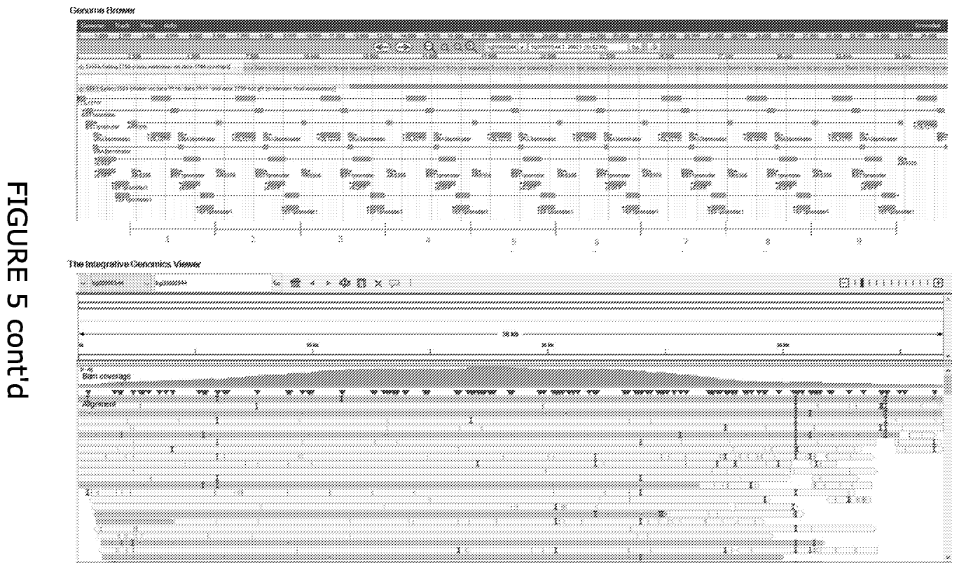

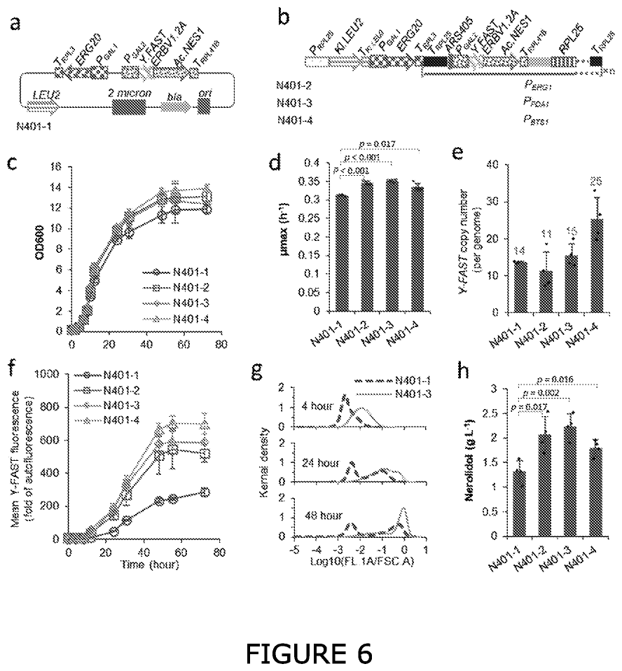

We constructed a reference strain N401-1 harboring a multi-copy 2μ plasmid pJT9RFR 38 (FIG. 6a) with overexpression cassettes for farnesyl pyrophosphate synthase (ERG20) and nerolidol synthase (Ac.NES1). The nerolidol synthase cassette includes a fluorescence-activating and absorption-shifting tag (Y-FAST) and a 2A peptide from Equine rhinitis B virus 1 fused to the N-terminus of nerolidol synthase. This allows Y-FAST fluorescence to be used as a proxy for nerolidol synthase expression.

The nerolidol synthase expression cassette (Y-FAST-2A-Ac.NES1) was cloned into the RPL25 insertion vector in the amplification region with three different promoters for replacement of the RPL25 promoter; the ERG20 expression cassette was cloned at the non-amplification region (FIG. 6b). Colonies with bright Y-FAST fluorescence were selected from the transformation plates. This delivered strains N401-2, N401-3, & N401-4 (promoters P_{ERG1}, P_{PDA1}, and P_{BTS1}, respectively).

Compared to the reference strain N401-1, these three strains exhibited faster growth (FIGS. 6c & 6d), higher Y-FAST fluorescence (FIG. 6f), and higher nerolidol production (FIG. 6h). The Y-FAST-2A-Ac.NES1 cassette was successfully amplified in vivo in the three test strains (FIG. 6e).

The reference 2μ plasmid strain harbored 14 copies of the Y-FAST-2A-AcNES1 construct-similar to strain N401-3, and higher than that in strain N401-2. However, N401-1 had the lowest Y-FAST fluorescence (FIG. 6f). The discrepancy between copy number and fluorescence was due to lack of induction of Y-FAST expression in a large proportion of N401-1 cells (FIG. 6g).

In contrast with the 2μ plasmid strain, the strains harboring the integrated in vivo amplification constructs showed better synchronicity for Y-FAST induction (FIG. 6g N401-3). This may contribute to the improved production.

### Example 6

### Improving Heterologous Production of the Monoterpene Limonene

The performance of the presently described genetic amplification strategy/method was tested with the production of C₁₀ monoterpenes. Monoterpene production requires introduction of a dedicated C₁₀ geranyl pyrophosphate (GPP) synthase (Ignea, C. et al. ACS synthetic biology (2013)). A previously used Erg20p^{N127W} mutant, which excludes the C15 chain from the active site to generate a GPP pool, in combination with targeted degradation of the endogenous C₁₅ synthase Erg20p via protein degron tags to decrease competition at the C₁₀ node by Erg20p and redirect GPP towards monoterpene production, was used. In mevalonate pathway-enhanced strains, this approach delivered less than 100 mg L⁻¹; an order of magnitude below the levels achieved for sesquiterpene engineering.

In these experiments, a mevalonate pathway-enhanced strain with the endogenous Erg20p under an auxin-inducible protein degradation mechanism (Lu, Z. et al. Nature communications 12, 1051 (2021)) was used as a background strain.

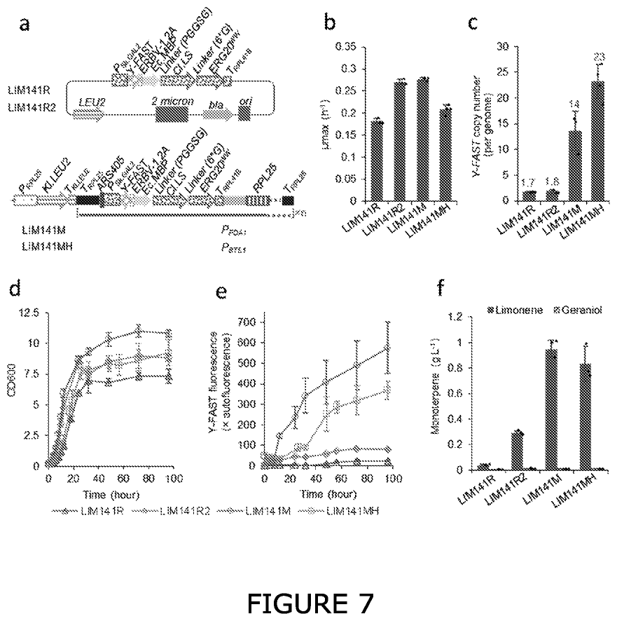

Two different promoter constructs were developed for amplification of the limonene synthetic module (FIG. 7a). The amplified region contained a fusion of multiple genes: Y-FAST-2A, the maltose-binding protein from E. coli for improved solubility, a short linker, limonene synthase from Citrus limon, a 6*glycerine linker, and a geranyl pyrophosphate synthase (the Erg20p N127W F96W mutant). This fusion construct was under the control of the GAL2 promoter from S. kudriavzevii. The two constructs were transformed into the RPL25 locus in the background strain, delivering strains LIM141M (P_{PDA1}) and LIM141MH (P_{BTS1}). The construct was introduced into the background strain via a 2μ plasmid. Four biological replicates were characterized (LIM141R representing three biological replicates and LIM141R2 representing one biological replicate; FIG. 7). In this case, 2μ plasmid delivered ˜2 copies per genome of the limonene synthase/Y-FAST module (shown by Y-FAST copy number; FIG. 7c). LIM141R, the three biological replicates produced ˜40 mg L⁻¹ limonene (FIG. 7f), similar to reports of a previous strain LIM141 expressing limonene synthase and Erg20p^{N127W} without gene fusion. LIM141R2 produced ˜300 mg L⁻¹ limonene.

Strain LIM141MH showed a slower exponential growth and the lower levels of Y-FAST fluorescence compared to strain LIM141M, despite having more copies of the limonene synthase module (FIG. 7).

Both strains produced an order of magnitude more limonene than over previous efforts using 2μ plasmids, producing ˜0.95 g L⁻¹ limonene at 96 hr, by strain LIM141M (FIG. 7e). This titer is 5.6-fold higher than the previous highest titer ever obtained in yeast, and ˜2-fold higher than the best titers achieved in batch cultivation in E. coli. Both strains also accumulated ˜12 mg L⁻¹ of the monoterpene alcohol geraniol, which is commonly produced by yeast with an increased GPP pool. This is about 45% less geraniol than when a 2μ plasmid is used. No farnesol (C₁₅ alcohol) or geranylgeraniol (C₂₀ alcohol) were accumulated by the strains, indicating that subcellular pools of FPP and the C₂₀ geranylgeranyl pyrophosphate (GGPP) were low, and that amplification of limonene synthetic module led to significant redirection of the carbon flux towards monoterpene production.

### Example 7

### Improving Heterologous Triterpenoid Lycopene Production in Yeast

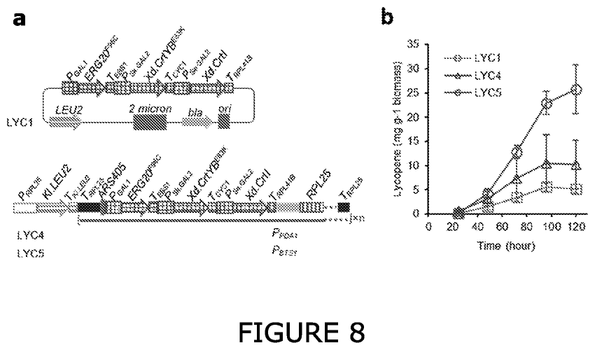

A three-gene lycopene synthetic module controlled by GAL promoters was previously constructed in a 2μ plasmid (FIG. 8a). This construct includes the farnesyl pyrophophase mutant gene ERG20^{F96C} which produces geranylgeranyl pyrophosphate, a phytoene synthase, and a lycopene-forming phytoene desaturase mutant. This plasmid was transformed into a mevalonate pathway-enhanced background strain, generating strain LYC1. This strain accumulated ˜5 mg lycopene per gram of biomass in 120-hour flask cultivation (FIG. 8b).

The lycopene synthetic module was sub-cloned into both the PDA1 and BTS1 promoter RPL25-driving HapAmp vectors (FIG. 8a). The resulting constructs were transformed into the same background strain, generating strains LYC4 and LYC5, respectively.

Strain LYC4 (P_{PDA1}-RPL25) accumulated slightly more lycopene than strain LYC1, although the increase was not significant (FIG. 7b). Strain LYC5 accumulated ˜25 mg lycopene per gram of biomass, 5-fold higher than strain LYC1 (FIG. 8b).

### Example 8

### High-Level Expression of Heterologous Proteins in Yeast

Yeast is commonly used as a platform organism for protein production, including production of pharmaceutical proteins, with the advantage of the lack of endotoxins. However, a notorious disadvantage is that heterologous proteins production is not as high as what is achievable with E. coli expression systems. The high-level expression in E. coli can be attributed to the usage of high-copy-number plasmids (such as the common pET vectors with copy number about ˜15˜20) and the use of a very strong inducible promoter.

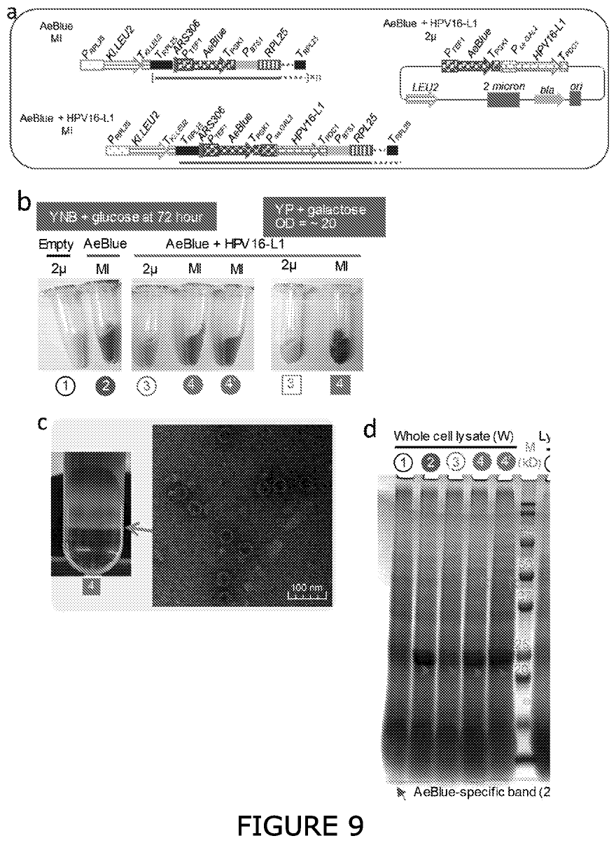

In the following experiments, the PBTS1-RPL25-driving genetic construct was used to introduce the AeBlue chromoprotein gene (FIG. 9a) or the EforRed chromoprotein gene. Blue or pink colonies were observed on the transformation plates, indicating high-level expression of the chromoproteins.

Having confirmed that the chromoproteins were effective markers, human papillomavirus (HPV) 16 major capsid protein L1 gene was inserted after the AeBlue expression cassette (FIG. 9a) to test the system for production of a pharmaceutical protein. For a reference, we cloned AeBlue-and-HPV16-L1 expression cassettes into a yeast 2μ plasmid (FIG. 9a). To compare the efficiency of protein production in different systems, an empty 2μ plasmid, the AeBlue-and-HPV16-L1 2μ plasmid, the RPL25-amplifiable AeBlue construct, and the RPL25-amplifiable AeBlue-and-HPV16-L1 construct were transformed individually into CEN.PK (gal80Δ). The four resulting strains were grown in MES-buffered YNB medium with 20 g L⁻¹ glucose aerobically for 72 hours.

Cells with multi-copy integration of the AeBlue expression cassette showed a strong Tibetan blue color, while cells with an empty cassette were milky white color (FIG. 9b). The cells with 2μ plasmid containing AeBlue+HPV-L1 expression cassettes were a faint blue color, whereas the cells with multi-copy integration of AeBlue+HPV-L1 expression cassettes displayed the strong Tibetan blue color (FIG. 9b). This indicated superior expression capacity from the in vivo amplification method for multi-copy genome integration, compared to conventional 2μ plasmid method.

SDS-PAGE analysis of whole cell and soluble protein extracts showed bands at ˜25 kD (AeBlue molecular weight) in all samples, with much stronger bands observed in the multi-copy integration strain samples than in the 2μ plasmid strain samples (FIG. 9d). In the multi-copy integration strains, these bands represented ˜3% of whole-cell protein, suggesting heterologous protein expression in yeast may reach the levels often obtained in E. coli.

A second strong band at ˜50 kD band (HPV16-L1 molecular weight) was observed in samples from cells expressing HPV-L1, although it was not as distinct at the putative AeBlue band (FIG. 9d). The expression of this transgene is under control of the the Se. GAL2 promoter, which is known to not be fully induced in the ethanol phase in these constructs, when compared to the constitutive ALD6 promoter used for the AeBlue expression cassette. Again, the bands in the multi-copy integration strain samples were stronger than the 2μ plasmid samples, and were clearly present in the VLP samples.

Disclosed herein is a novel genetic engineering method to integrate multiple copies of heterologous gene(s) into the yeast genome using in vivo gene amplification driven by a haploinsufficient gene. The functional strength per copy of a haploinsufficient gene is strongly associated with growth fitness, which can be exploited as an evolutionary force to drive gene amplification. Decreased expression level provides an evolutionary force that drives amplification of linked haploinsufficient and heterologous genes, so that cells are growth-competitive.

Provided here are examples of the application of this method to improve production of different types of terpene products, however the application of this method is not limited to the terpene products. Also shown is that the present method can be used to enable high-level expression of any other heterologous protein in yeast, at levels similar to that achieved in E. coli for protein production.

This method advantageous for the introduction of heterologous genes via genome integration. Firstly, integration copy number can be titrated by altering the expression dosage per copy of haploinsufficient gene. Expression level can be reduced by a variety of methods, including but not limited to (1) replacing the gene promoter with a weaker promoter, and (2) using non-preferred codons.

Amplification efficiency observed was 4 to 47 copies of the heterologous genes, with an inverse relationship between promoter strength and copy number. However, it can be easily recognized that suitable alteration of the expression dosage of the haploinsufficiency gene will drive less or more amplification.

A number of weak promoters are described herein (Table 1 and FIG. 2) and in previous work (Peng, B. et al. Microbial cell factories 14, 91 (2015)) that can be applied to decrease gene dosage. In addition to promoter strength and codon usage, other approaches could be used to decrease expression dosage, including engineering the Kozak sequence and/or the 5′-mRNA structure. These genetic tools add engineering flexibility to modify copy number for this HapAmp method in yeast.

Another advantage is that the maintenance of integration is auto-selectable: selection pressure is provided from the dosage sensitivity of the haploinsufficient gene, which is linked to the gene of interest and is maintained to support normal growth rates. This means that no antibiotics or modification of other environmental conditions in the culture are required to provide ongoing selection pressure for maintenance of the gene of interest. Compared to use of a 2μ plasmid, this method provides for improved stable expression of heterologous proteins in yeast (FIG. 9b). In addition, it does not require chemical induction for gene amplification.

The presence of multiple haploinsufficient genes within a host cell genome means that many different loci are available for engineering gene amplification. Characterization of the promoter strength of fifteen additional haploinsufficient genes provided here (Table 1) can also be used to drive gene amplification.

Initial integration of the genes of interest uses standard yeast transformation procedures by selection of an auxotrophic or antibiotic marker (e.g., LEU2 or hphMax). Use of visual markers (fluorescent proteins or chromoproteins) can facilitate the selection of correct clones with amplified constructs.

The present disclosure disclosed herein successfully improved production of heterologous terpenes including the C₁₅ sesquiterpene nerolidol (FIG. 4), the C₁₀ monoterpene limonene (FIG. 7), and the C₃₀ triterpene lycopene (FIG. 8).

Production of C₁₅ terpenes in yeast is typically relatively straightforward, with g L⁻¹ titres achievable. The C₁₅ precursor, FPP, is produced in yeast naturally to deliver sterol pathway products required for yeast growth. In addition, sesquiterpene synthases have reasonably good catalytic properties, making them more competitive to access FPP.

However production of C₁₀ monoterpenes, however, has historically been very challenging. This is due to both a dearth of C₁₀ precursors and the poor catalytic properties of many monoterpene synthases. These limitations have previously restricted published titers of monoterpenes to mg L⁻¹ in flask cultivation. Here, we have achieved g L⁻¹ titers (FIG. 7) in a single engineering step using a high mevalonate pathway flux strain with an introduced GPPS and targeted degradation of FPPS to decrease competition at the C10 pathway node. At present, this is the highest titre achieved in metabolically engineered microbes in a flask cultivation with 20 g L⁻¹ glucose as carbon source reported to date.

Variation in the different systems results in variable improvement ratios, for example, limonene production improvement was ˜20-fold, whereas nerolidol improvement was 1.7-fold, and lycopene improvement was 5-fold. However a higher titer is seen with in vivo gene amplification. In particular, for monoterpenes, insufficient catalytic efficiency of terpene synthase is a significant bottleneck for production of heterologous terpenoids in yeast. Increasing copy number via insertion of tandem repeats at the same locus combined with screening for improved production or introduction of additional expression cassettes at separate loci has been used to overcome this bottleneck previously. However, these approaches require complex cloning and extended experimental timelines to deliver the desired improvements. The presently disclosed disclosure advantageously provides means to overcome these challenges by providing a faster and simpler method to achieve superior results.

In addition to its application in metabolic engineering, the presently disclosure can be used for increasing heterologous protein production. Using chromoprotein AeBlue and the HPV16 L1 capsid protein as examples (FIG. 9), it was demonstrated that in S. cerevisiae, heterologous protein could be produced at levels commonly seen in E. coli.

The presently disclosed method is applicable to other industrially relevant chassis organisms that have haploinsufficient genes. A potential haploinsufficient gene may encode essential components of the machineries for protein synthesis and transportation or other essential cell structures. Putative haploinsufficient genes can be identified by comparative genomics and confirmed by testing growth fitness in association with expression dosage of a gene.

TABLE 2
Plasmids used

| Plasmid | Properties |
|---|---|
| pILGFP3 | Yeast integration plasmid; P_{URA3} > KI.URA3 > T_{KI.URA3}- yEGFP > T_{URA3} |
| pILGFP1D5 | Yeast integration plasmid; P_{URA3} > KI.URA3 > T_{KI.URA3}- yEGFP > T_{PGK1}-T_{URA3} |
| pILGFP5A3 | Yeast integration plasmid; P_{URA3} > KI.URA3 > T_{KI.URA3}-P_{YEF3} > yEGFP > T_{PGK1}-T_{URA3} |
| pILGFP1A6 | Yeast integration plasmid; P_{URA3} > KI.URA3 > T_{KI.URA3}-P_{RPL25} > yEGFP > T_{PGK1}-T_{URA3} |
| pILGFP1C6 | Yeast integration plasmid; P_{URA3} > KI.URA3 > T_{KI.URA3}-P_{SEC23} > yEGFP > T_{PGK1}-T_{URA3} |
| pILGFP1E6 | Yeast integration plasmid; P_{URA3} > KI.URA3 > T_{KI.URA3}-P_{PDA1} > yEGFP > T_{PGK1}-T_{URA3} |
| pILGFP1E7 | Yeast integration plasmid; P_{URA3} > KI.URA3 > T_{KI.URA3}-P_{ERG1} > yEGFP > T_{PGK1}-T_{URA3} |
| pILGFP1G7 | Yeast integration plasmid; P_{URA3} > KI.URA3 > T_{KI.URA3}-P_{BTS1} > yEGFP > T_{PGK1}-T_{URA3} |
| pILGFP4F5 | Yeast integration plasmid; P_{URA3} > KI.URA3 > T_{KI.URA3}-P_{GLO2} > yEGFP > T_{PGK1}-T_{URA3} |
| pILGFP4H5 | Yeast integration plasmid; P_{URA3} > KI.URA3 > T_{KI.URA3}-P_{COG7} > yEGFP > T_{PGK1}-T_{URA3} |
| pILGFP89 | Yeast integration plasmid; P_{URA3} > KI.URA3 > T_{KI.URA3}- P_{TEF1} > yEGFP > T_{URA3} |
| pILGFP1DFB | Yeast integration plasmid; P_{RPL25}(Arm 1) > KI.LEU2 > T_{KI.LEU2-}T_{RPL25}(Arm 3)- |
| | ARS305-P_{TEF1} > yEGFP > T_{URA3} |
| pILGFP3A5C | Yeast integration plasmid; P_{RPL25}(Arm 1) > KI.LEU2 > T_{KI.LEU2-}T_{RPL25}(Arm 2)- |
| | ARS305-P_{TEF1} > yEGFP > T_{URA3}- P_{YEF3} > RPL25(partial; Arm3) |
| pILGFP3AE4 | Yeast integration plasmid; P_{RPL25}(Arm 1) > KI.LEU2 > T_{KI.LEU2-}T_{RPL25}(Arm 3)- |
| | ARS305-P_{TEF1} > yEGFP > T_{URA3}- P_{ERG1} > RPL25(partial; Arm2) |
| pILGFP3AG4 | Yeast integration plasmid; P_{RPL25}(Arm 1) > KI.LEU2 > T_{KI.LEU2-}T_{RPL25}(Arm 3)- |
| | ARS305-P_{TEF1} > yEGFP > T_{URA3}- P_{PDA1} > RPL25(partial; Arm2) |
| pILGFP3AA5 | Yeast integration plasmid; P_{RPL25}(Arm 1) > KI.LEU2 > T_{KI.LEU2-}T_{RPL25}(Arm 3)- |
| | ARS305-P_{TEF1} > yEGFP > T_{URA3}- P_{BTS1} > RPL25(partial; Arm2) |
| pILGFP3AG4ARSd | Yeast integration plasmid; P_{RPL25}(Arm 1) > KI.LEU2 > T_{KI.LEU2-}T_{RPL25}(Arm 3)- |
| | P_{TEF1} > yEGFP > T_{URA3}- P_{PDA1} > RPL25(partial; Arm2) |
| pILGFP4BG6 | Yeast integration plasmid; P_{SEC23}(Arm 1) > P_{Ag.TEF1} > hphMX4 > T_{Ag.TEF1}- |
| | T_{SEC23}(Arm 3)-ARS1max-P_{TEF1} > yEGFP > T_{URA3} |
| pILGFP5EG3 | Yeast integration plasmid; P_{SEC23}(Arm 1) > P_{Ag.TEF1} > hphMX4 > T_{Ag.TEF1}- |
| | T_{SEC23}(Arm 3)-ARS1max-P_{TEF1} > yEGFP > T_{URA3}-P_{ERG1} > SEC23(partial; Arm2) |
| pILGFP5EA4 | Yeast integration plasmid; P_{SEC23}(Arm 1) > P_{Ag.TEF1} > hphMX4 > T_{Ag.TEF1}- |
| | T_{SEC23}(Arm 3)-ARS1max-P_{TEF1} > yEGFP > T_{URA3}-P_{GLO2} > SEC23(partial; Arm2) |
| pILGFP5EC4 | Yeast integration plasmid; P_{SEC23}(Arm 1) > P_{Ag.TEF1} > hphMX4 > T_{Ag.TEF1}- |
| | T_{SEC23}(Arm 3)-ARS1max-P_{TEF1} > yEGFP > T_{URA3}-P_{COG7} > SEC23(partial; Arm2) |
| pILGFP5EF3 | Yeast integration plasmid; P_{SEC23}(Arm 1) > P_{Ag.TEF1} > hphMX4 > T_{Ag.TEF1}- |
| | T_{SEC23}(Arm 3)-ARS1max-P_{TEF1} > yEGFP > T_{URA3}-P_{COG7} > ATGGGAGGAGGA- |
| | SEC23(partial; Arm2) |
| pILGFP6G3 | Yeast integration plasmid; P_{URA3} > KI.URA3 > T_{KI.URA3}-P_{RPL33A} > yEGFP > T_{PGK1}-T_{URA3} |
| pILGFP6A4 | Yeast integration plasmid; P_{URA3} > KI.URA3 > T_{KI.URA3}-P_{RPS15} > yEGFP > T_{PGK1}-T_{URA3} |
| pILGFP6C4 | Yeast integration plasmid; P_{URA3} > KI.URA3 > T_{KI.URA3}-P_{RPC10} > yEGFP > T_{PGK1}-T_{URA3} |
| pACT1-GFP | Yeast integration plasmid; P_{URA3} > KI.URA3 > T_{KI.URA3}-P_{ACT1} > yEGFP > T_{PGK1}-T_{URA3} |
| pILGFP6G4 | Yeast integration plasmid; P_{URA3} > KI.URA3 > T_{KI.URA3}-P_{NIP1} > yEGFP > T_{PGK1}-T_{URA3} |
| pILGFP6A5 | Yeast integration plasmid; P_{URA3} > KI.URA3 > T_{KI.URA3}-P_{RPS13} > yEGFP > T_{PGK1}-T_{URA3} |
| pILGFP6C5 | Yeast integration plasmid; P_{URA3} > KI.URA3 > T_{KI.URA3}-P_{NUS1} > yEGFP > T_{PGK1}-T_{URA3} |
| pILGFP6E5 | Yeast integration plasmid; P_{URA3} > KI.URA3 > T_{KI.URA3}-P_{SMC1} > yEGFP > T_{PGK1}-T_{URA3} |
| pILGFP6G5 | Yeast integration plasmid; P_{URA3} > KI.URA3 > T_{KI.URA3}-P_{RNA14} > yEGFP > T_{PGK1}-T_{URA3} |
| pILGFP6A6 | Yeast integration plasmid; P_{URA3} > KI.URA3 > T_{KI.URA3}-P_{RPB7} > yEGFP > T_{PGK1}-T_{URA3} |
| pILGFP6C6 | Yeast integration plasmid; P_{URA3} > KI.URA3 > T_{KI.URA3}-P_{SPC97} > yEGFP > T_{PGK1}-T_{URA3} |
| pILGFP6E6 | Yeast integration plasmid; P_{URA3} > KI.URA3 > T_{KI.URA3}-P_{STH1} > yEGFP > T_{PGK1}-T_{URA3} |
| pILGFP6G6 | Yeast integration plasmid; P_{URA3} > KI.URA3 > T_{KI.URA3}-P_{ARP7} > yEGFP > T_{PGK1}-T_{URA3} |
| pILGFP6A7 | Yeast integration plasmid; P_{URA3} > KI.URA3 > T_{KI.URA3}-P_{TAF61} > yEGFP > T_{PGK1}-T_{URA3} |
| pILGFP6C7 | Yeast integration plasmid; P_{URA3} > KI.URA3 > T_{KI.URA3}-P_{RPN11} > yEGFP > T_{PGK1}-T_{URA3} |
| pRS425 | E. coli/S. cerevisiae shuttle plasmid; 2μ, LEU2 |
| pIR3DH8 | Yeast integration plasmid; gal80Arm1-P_{AgTEF1}-KIURA3-T_{AgTEF1}-gal80Arm2 |
| pJT9RFR | pRS425 derivative; T_{RPL3} < ScERG20 < P_{GAL1}-P_{GAL2} > Y.FAST-EVBR1.2A- |
| | AcNES1 > T_{RPL41B} |
| pINER2R | pILGFP3AE4 derivative; P_{RPL25}(Arm 1) > KI.LEU2 > T_{KI.LEU2}-P_{GAL1} > ERG20 > T_{RPL3}- |
| | T_{RPL25}(Arm 3)- ARS305- P_{GAL2} > Y.FAST-EVBR1.2A-AcNES1 > T_{RPL41B} > |
| | RPL25(partial; Arm2) |
| pINER3R | pILGFP3AG4 derivative; P_{RPL25}(Arm 1) > KI.LEU2 > T_{KI.LEU2}-P_{GAL1} > ERG20 > T_{RPL3}- |
| | T_{RPL25}(Arm 3)- ARS305- P_{GAL2} > Y.FAST-EVBR1.2A-AcNES1 > T_{RPL41B} -P_{PDA1} > |
| | RPL25(partial; Arm2) |
| pINER4R | pILGFP3AA5 derivative; P_{RPL25}(Arm 1) > KI.LEU2 > T_{KI.LEU2}-P_{GAL1} > ERG20 > T_{RPL3}- |
| | T_{RPL25}(Arm 3)- ARS305- P_{GAL2} > Y.FAST-EVBR1.2A-AcNES1 > T_{RPL41B} - P_{BTS1} > |
| | RPL25(partial; Arm2) |
| pIT6EG7m | pILGFP3AG4 derivative; P_{RPL25}(Arm 1) > KI.LEU2 > T_{KI.LEU2}- T_{RPL25}(Arm 3)- |
| | ARS305- P_{Sk.GAL2} > Y.FAST-EVBR1.2A-Ec.MBP-Linker~SacI~6*G-ERG20^{F96W N127W} > |
| | T_{RPL3} -P_{PDA1} > RPL25(partial; Arm2) |
| pIT6EG7ml | pILGFP3AG4 derivative; P_{RPL25}(Arm 1) > KI.LEU2 > T_{KI.LEU2}- T_{RPL25}(Arm 3)- |
| | ARS305- P_{Sk.GAL2} > Y.FAST-EVBR1.2A-Ec.MBP-Linker-LI.LS-6*G-ERG20^{F96W N127W} > |
| | T_{RPL3}-P_{PDA1} > RPL25(partial; Arm2) |
| pIT6EG7mlh | pILGFP3AA5 derivative; P_{RPL25}(Arm 1) > KI.LEU2 > T_{KI.LEU2}- T_{RPL25}(Arm 3)- |
| | ARS305- P_{Sk.GAL2} > Y.FAST-EVBR1.2A-Ec.MBP-Linker-LI.LS-6*G-ERG20^{F96W N127W} > |
| | T_{RPL3} -P_{BTS1} > RPL25(partial; Arm2) |
| pPT6EG7ml | pRS425 derivative; P_{Sk.GAL2} > Y.FAST-EVBR1.2A-Ec.MBP-Linker~SacI~6*G- |
| | ERG20^{F96W N127W} > T_{RPL3} |
| pLAC1 | pRS425 derivative; P_{GAL1} > ERG20^{F96C} > T_{EBS1}-P_{Sk.GAL2} > Xd.CRtYB^{E83K} > T_{CYC1}- |
| | P_{Se.GAL2} > XdCrtI > T_{RPL41B} |
| pILAC2 | pILGFP3AG4 derivative; P_{RPL25}(Arm 1) > KI.LEU2 > T_{KI.LEU2}- T_{RPL25}(Arm 3)- |
| | ARS305- P_{GAL1} > ERG20^{F96C} > T_{EBS1}-P_{Sk.GAL2} > Xd.CRtYB^{E83K} > T_{CYC1}- |
| | P_{Se.GAL2} > XdCrtI > T_{RPL41B} -P_{PDA1} > RPL25(partial; Arm2) |
| pILAC3 | pILGFP3AA5 derivative; P_{RPL25}(Arm 1) > KI.LEU2 > T_{KI.LEU2}- T_{RPL25}(Arm 3)- |
| | ARS305- P_{GAL1} > ERG20^{F96C} > T_{EBS1}-P_{Sk.GAL2} > Xd.CRtYB^{E83K} > T_{CYC1}- |
| | P_{Se.GAL2} > XdCrtI > T_{RPL41B} -P_{BTS1} > RPL25(partial; Arm2) |
| pIAeBlue | pILGFP3AA5 derivative; P_{RPL25}(Arm 1) > KI.LEU2 > T_{KI.LEU2}- T_{RPL25}(Arm 3)- |
| | ARS305- P_{ALD6} > AeBlue > T_{PGK1}- P_{BTS1} > RPL25(partial; Arm2) |
| pIEforRed | pILGFP3AA5 derivative; P_{RPL25}(Arm 1) > KI.LEU2 > T_{KI.LEU2}- T_{RPL25}(Arm 3)- |
| | ARS305- P_{ALD6} > EforRed > T_{PGK1}- P_{BTS1} > RPL25(partial; Arm2) |
| pIR3DH8K | Yeast integration plasmid; gal80Arm1-P_{TPI1}-KanMX4-gal80Arm2 |
| pPAeBlueHPV16LR | pRS425 derivative; P_{ALD6} > AeBlue > T_{PGK1}- P_{Se.GAL2} > HPV16-L1ΔC-6*H > |
| | T_{RPL41B} |
| pIAeBlueHPV16LR | pILGFP3AA5 derivative; P_{RPL25}(Arm 1) > KI.LEU2 > T_{KI.LEU2}- T_{RPL25}(Arm 3)- |
| | ARS305- P_{ALD6} > EforRed > T_{PGK1}- P_{Se.GAL2} > HPV16-L1ΔC-6*H > T_{RPL41B}-P_{BTS1} > |
| | RPL25(partial; Arm2) |

TABLE 3
Saccharomyces cerevisiae strains used in this work

| Strain | Genotype |
|---|---|
| CEN.PK2-1C | MATa ura3-52 trp1-289 leu2-3, 112 his3Δ 1 |
| CEN.PK113- | MATa ura3-52 |
| 5D | |
| CEN.PK113- | MATa leu2-3 |
| 16B | |
| CEN.PK113- | MATa |
| 7D | |

ILHA series strains

| | |
|---|---|
| GH4 | CEN.PK113-5D derivative; ura3(1, 704)::KI.URA3 > T_{KI.URA3} |
| G5A3 | CEN.PK113-5D derivative; ura3(1, 704):: KI.URA3 > T_{KI.URA3}- P_{YEF3} > yEGFP > T_{PGK1} |
| | (FIG. 2d) |
| G1A6 | CEN.PK113-5D derivative; ura3(1, 704):: KI.URA3 > T_{KI.URA3}- P_{RPL25} > yEGFP > T_{PGK1} |
| | (FIG. 2d) |
| G1C6 | CEN.PK113-5D derivative; ura3(1, 704):: KI.URA3 > T_{KI.URA3}- P_{SEC23} > yEGFP > T_{PGK1} |
| | (FIG. 2d) |
| G1E6 | CEN.PK113-5D derivative; ura3(1, 704):: KI.URA3 > T_{KI.URA3}- P_{PDA1} > yEGFP > T_{PGK1} |
| | (FIG. 2d) |
| G1E7 | CEN.PK113-5D derivative; ura3(1, 704):: KI.URA3 > T_{KI.URA3}- P_{ERG1} > yEGFP > T_{PGK1} |
| | (FIG. 2d) |
| G1G7 | CEN.PK113-5D derivative; ura3(1, 704):: KI.URA3 > T_{KI.URA3}- P_{BTS1} > yEGFP > T_{PGK1} |
| | (FIG. 2d) |
| G4F5 | CEN.PK113-5D derivative; ura3(1, 704):: KI.URA3 > T_{KI.URA3}- P_{GLO2} > yEGFP > T_{PGK1} |
| | (FIG. 2d) |
| G4H5 | CEN. PK113-5D derivative; ura3(1, 704):: KI.URA3 > T_{KI.URA3}- P_{COG7} > yEGFP > T_{PGK1} |
| | (FIG. 2d) |
| G3A5C | CEN.PK113-16B derivative; RPL25:: KI.LEU2 > T_{KI.LEU2}-T_{RPL25}- ARS305-P_{TEF1} > |
| | yEGFP > T_{URA3}- P_{YEF3}-RPL25 |
| | (FIG. 2, Construct 1) |
| G3AE4 | CEN.PK113-16B derivative; RPL25:: KI.LEU2 > T_{KI.LEU2-{}T_{RPL25}- ARS305-P_{TEF1} > |
| | yEGFP > T_{URA3}- P_{ERG1}-RPL25}_{×n} |
| | (FIG. 2, Construct 2) |
| G3AG4 | CEN.PK113-16B derivative; RPL25:: KI.LEU2 > T_{KI.LEU2-{}T_{RPL25}- ARS305-P_{TEF1} > |
| | yEGFP > T_{URA3}- P_{PDA1}-RPL25}_{×n} |
| | (FIG. 2, Construct 3) |
| G3AA5 | CEN.PK113-16B derivative; RPL25:: KI.LEU2 > T_{KI.LEU2-{}T_{RPL25}- ARS305-P_{TEF1} > |
| | yEGFP > T_{URA3}- P_{BTS1}-RPL25}_{×n} |
| | (FIG. 2, Construct 4) |
| G5EG3 | CEN.PK113-7D derivative; SEC23:: P_{Ag.TEF1} > hphMX4 > T_{Ag.TEF1}- T_{SEC23}-ARS1max- |
| | P_{TEF1} > yEGFP > T_{URA3}-P_{ERG1} > SEC23 |
| | (FIG. 2, Construct 5) |
| G5EA4 | CEN.PK113-7D derivative; SEC23:: P_{Ag.TEF1} > hphMX4 > T_{Ag.TEF1}- {T_{SEC23}-ARS1max- |
| | P_{TEF1} > yEGFP > T_{URA3}-P_{GLO2} > SEC23}CT_{×n} |
| | (FIG. 2, Construct 6) |
| G5EC4 | CEN.PK113-7D derivative; SEC23:: P_{Ag.TEF1} > hphMX4 > T_{Ag.TEF1}- {T_{SEC23}-ARS1max- |
| | P_{TEF1} > yEGFP > T_{URA3}-P_{COG7} > SEC23}_{×n} |
| | (FIG. 2, Construct 7) |
| G5EF3 | CEN.PK113-7D derivative; SEC23:: P_{Ag.TEF1} > hphMX4 > T_{Ag.TEF1}- {T_{SEC23}-ARS1max- |
| | P_{TEF1} > yEGFP > T_{URA3}-P_{COG7} > ATGGGAGGAGGA-SEC23}_{×n} |
| | (FIG. 2, Construct 8) |
| G6G3 | CEN.PK113-5D derivative; ura3(1, 704):: KI.URA3 > T_{KI.URA3}- P_{RPL33A} > yEGFP > T_{PGK1} |
| | (FIG. S2) |
| G6A4 | CEN.PK113-5D derivative; ura3(1, 704):: KI.URA3 > T_{KI.URA3}- P_{RPS15} > yEGFP > T_{PGK1} |
| | (FIG. S2) |
| G6C4 | CEN.PK113-5D derivative; ura3(1, 704):: KI.URA3 > T_{KI.URA3}- P_{RPC10} > yEGFP > T_{PGK1} |
| | (FIG. S2) |
| GATC1-GFP | CEN.PK113-5D derivative; ura3(1, 704):: KI.URA3 > T_{KI.URA3}- P_{ACT1} > yEGFP > T_{PGK1} |
| | (FIG. S2) |
| G6G4 | CEN.PK113-5D derivative; ura3(1, 704):: KI.URA3 > T_{KI.URA3}- P_{NIP1} > yEGFP > T_{PGK1} |
| | (FIG. S2) |
| G6A5 | CEN.PK113-5D derivative; ura3(1, 704):: KI.URA3 > T_{KI.URA3}- P_{RPS13} > yEGFP > T_{PGK1} |
| | (FIG. S2) |
| G6C5 | CEN.PK113-5D derivative; ura3(1, 704):: KI.URA3 > T_{KI.URA3}- P_{NUS1} > yEGFP > T_{PGK1} |
| | (FIG. S2) |
| G6E5 | CEN.PK113-5D derivative; ura3(1, 704):: KI.URA3 > T_{KI.URA3}- P_{SMC1} > yEGFP > T_{PGK1} |
| | (FIG. S2) |
| G6G5 | CEN.PK113-5D derivative; ura3(1, 704):: KI.URA3 > T_{KI.URA3}- P_{RNA1} > yEGFP > T_{PGK1} |
| | (FIG. S2) |
| G6A6 | CEN.PK113-5D derivative; ura3(1, 704):: KI.URA3 > T_{KI.URA3}- P_{RPB7} > yEGFP > T_{PGK1} |
| | (FIG. S2) |
| G6C6 | CEN.PK113-5D derivative; ura3(1, 704):: KI.URA3 > T_{KI.URA3}- P_{SPC97} > yEGFP > T_{PGK1} |
| | (FIG. S2) |
| G6E6 | CEN.PK113-5D derivative; ura3(1, 704):: KI.URA3 > T_{KI.URA3}- P_{STH1} > yEGFP > T_{PGK1} |
| | (FIG. S2) |
| G6G6 | CEN.PK113-5D derivative; ura3(1, 704):: KI.URA3 > T_{KI.URA3}- P_{ARP7} > yEGFP > T_{PGK1} |
| | (FIG. S2) |
| G6A7 | CEN.PK113-5D derivative; ura3(1, 704):: KI.URA3 > T_{KI.URA3}- P_{TAF61} > yEGFP > T_{PGK1} |
| | (FIG. S2) |
| G6C7 | CEN.PK113-5D derivative; ura3(1, 704):: KI.URA3 > T_{KI.URA3}- P_{RPN11} > yEGFP > T_{PGK1} |
| | (FIG. S2) |
| o401R | CEN.PK2-1C derivative; |
| | HMG2^{K6R}(−152, −1)::HIS3-T_{EFM1} < EfmvaS < P_{GAL1}-P_{GAL10} > ACS2 > T_{ACS2}- |
| | P_{GAL2} > EfmvaE > T_{EBS1}-P_{GAL7} |
| | pdc5 (−31, 94)::P_{GAL2} > ERG12 > T_{NAT5}-P_{TEF2} > ERG8 > T_{IDP1}- |
| | T_{PRM9} < MVD1 < P_{ADH2}-T_{RPL15A} < IDI1 < P_{TEF1}-TRP1 |
| | ERG9(1333, 1335)::T_{URA3}- P_{GAL7} > MVD1 > T_{PRM9}-P_{GAL2} > ERG12 > T_{NAT5}- |
| | T_{IDP1} < ERG8 < P_{GAL10}-P_{GAL1} > IDI1 > T_{RPL15A}-loxP-ble-loxP |
| o401UR | o401R derivative; |
| | gal80::P_{AgTEF1} > KI.URA3 > T_{AgTEF1} |
| N401-1 | o401UR derivative; |
| | [pJT9RFR] |
| N401-2 | o401UR derivative; |
| | RPL25:: KI.LEU2 > T_{KI.LEU2}-P_{GAL1} > ERG20 > T_{RPL3}-{T_{RPL25}- ARS305- P_{GAL2} > Y.FAST- |
| | EVBR1.2A-AcNES1 > T_{RPL41B} - P_{ERG1}-RPL25}_{×n} |
| N401-3 | o401UR derivative; |
| | RPL25:: KI.LEU2 > T_{KI.LEU2}-P_{GAL1} > ERG20 > T_{RPL3}-{T_{RPL25}- ARS305- P_{GAL2} > Y.FAST- |
| | EVBR1.2A-AcNES1 > T_{RPL41B} - P_{PDA1}-RPL25}_{×n} |
| N401-4 | o401UR derivative; |
| | RPL25:: KI.LEU2 > T_{KI.LEU2}-P_{GAL1} > ERG20 > T_{RPL3}-{T_{RPL25}- ARS305- P_{GAL2} > Y.FAST- |
| | EVBR1.2A-AcNES1 > T_{RPL41B} - P_{BTS1}-RPL25}_{×n} |
| o141R | o401R derivative; |
| | ERG20(−32, 3)::CUP1-AID* |
| | ura3(1, 704)::KI.URA3-T_{PGK1}-P_{ACS2} > SKP1-OsTIR1 |
| | gal80::P_{AgTEF1} > KanMX4 > T_{AgTEF1} |
| | ura3(1, 704)::KIURA3-T_{PGK1}-P_{ACS2} > SKP1-OsTIR1 |
| LIM141M | o141R derivative; |
| | RPL25:: KI.LEU2 > T_{KI.LEU2} -{T_{RPL25}- ARS305- P_{Sk.GAL2} > Y.FAST-EVBR1.2A-Ec.MBP- |
| | Linker~SacI~6*G-ERG20^{F96W N127W} > T_{RP1418} - P_{PDA1}-RPL25}_{×n} |
| | gal80::P_{AgTEF1} > KanMX4 > T_{AgTEF1} |
| LIM141MH | o141R derivative; |
| | RPL25:: KI.LEU2 > T_{KI.LEU2} -{T_{RPL25}- ARS305- P_{Sk.GAL2} > Y.FAST-EVBR1.2A-Ec.MBP- |
| | Linker~SacI~6*G-ERG20^{F95W N127W} > T_{RP141B} - P_{BTS1}-RPL25}_{×n} |
| | gal80::P_{AgTEF1} > KanMX4 > T_{AgTEF1} |
| LAC1 | o401R derivative; |
| | [pLAC1] |
| | gal80::P_{AgTEF1} > KanMX4 > T_{AgTEF1} |
| LAC4 | o401UR derivative; |
| | RPL25:: KI.LEU2 > T_{KI.LEU2} -{T_{RPL25}- ARS305- P_{GAL1} > ERG20^{F96C} > T_{EBS1}- |
| | P_{SK.GAL2} > Xd.CRtYB^{E83K} > T_{CYC1}-P_{Se.GAL2} > XdCrtI > T_{RPL41B} - P_{PDA1}-RPL25}_{×n} |
| LAC5 | o401UR derivative; |
| | RPL25:: KI.LEU2 > T_{KI.LEU2} -{T_{RPL25}- ARS305- P_{GAL1} > ERG20^{F96C} > T_{EBS1}- |
| | P_{Sk.GAL2} > Xd.CRtYB^{E83K} > T_{CYC1}-P_{Se.GAL2} > XdCrtI > T_{RPL41B} - P_{BTS1}-RPL25}_{×n} |
| 16BJ3 | CEN.PK113-16B derivative; |
| | gal80::P_{AgTEF1} > KanMX4 > T_{AgTEF1} |
| 16BJ3C | 16BJ3 derivative; |
| | [pRS425] |
| | (FIG. 6; Empty, 2μ) |
| 16BJ3AeBlue | 16BJ3 derivative; |
| | RPL25:: KI.LEU2 > T_{KI.LEU2}-P_{GAL1} > ERG20 > T_{RPL3}-{T_{RPL25}- ARS305- |
| | P_{ALD6} > AeBlue > T_{PGK1}- P_{BTS1}-RPL25}_{×n} |
| | (FIG. 6; AeBlue, MI) |
| HPV16LPR | 16BJ3 derivative; |
| | [pPAeBlueHPV16LR] |
| | (FIG. 6; AeBlue + HPV16-L1, 2μ) |
| HPV16LMR | 16BJ3 derivative; |
| | RPL25:: KI.LEU2 > T_{KI.LEU2}-P_{GAL1} > ERG20 > T_{RPL3}-{T_{RPL25}- ARS305- |
| | P_{ALD6} > AeBlue > T_{PGK1}- P_{Se.GAL2} > HPV16-L1ΔC-6*H > T_{RPL41B}-P_{BTS1}-RPL25}_{×n} |
| | (FIG. 6; AeBlue + HPV16-L1, MI) |

TABLE 4
List of primers and DNA fragments used in this work. P_{XXX} and T_{XXX} indicate promoter and
terminator sequence of gene XXX, respectively; italicized and underlined indicate sequences
complementary to the DNA template.

| SEQ | Overlap | | | |
|---|---|---|---|---|
| ID | extension | PCR/gBlock | | |
| No: | PCR fragment | fragment | Primer name | Sequence (5′→3′) |
| 1 | | T_{PGK1} from | PPGPGK1ts | GGATGAATTGTACAAAAGATCTTAAATTGA |
| | | SGD | | ATTGAATTGAAATCGATAG |
| 2 | | | PPGPGK1ta | CCCTTTGCAAATAGTCCTACTAGT |
| | | | | AAATAATATCCTTCTCGAAAGC |
| 3 | | P_{YEF3} from | PPGYEF3ps | AAGGGTTGCTCGAGAAAGAGCTC |
| | | SGD | | ATACATAACATTTTAAGATAAGCAAGTG |
| 4 | | | PPGYEF3pa | TGAATAATTCTTCACCTTTAGACAT |
| | | | | CTTTTAATGTTATCGATGGATTC |
| 5 | | P_{RPL25} from | PPGRPL25ps | AAGGGTTGCTCGAGAAAGAGCTC |
| | | SGD | | TCTTATCTTGTATGCCCGATAT |
| 6 | | | PPGRPL25pa | TGAATAATTCTTCACCTTTAGACAT |
| | | | | TTTATCTTATTGATCTTCTTTGTTTA |
| 7 | | P_{SEC23} from | PPGSEC23ps | AAGGGTTGCTCGAGAAAGAGCTC |
| | | SGD | | TGTCTTGTTGTGTTGTGACG |
| 8 | | | PPGSEC23pa | TGAATAATTCTTCACCTTTAGACAT |
| | | | | GGCTAGAAAAGAGGAAGGG |
| 9 | | P_{PDA1} from | PPGPDA1ps | AAGGGTTGCTCGAGAAAGAGCTC |
| | | SGD | | GAAATTCAAAACTCTCCAGAC |
| 10 | | | PPGPDA1pa | TGAATAATTCTTCACCTTTAGACAT |
| | | | | TGGCACAAATGTGGTTTCC |
| 11 | | P_{ERG1} from | PPGERG1ps | AAGGGTTGCTCGAGAAAGAGCTC |
| | | SGD | | TGCGATACTGCCGTAGCG |
| 12 | | | PPGERG1pa | TGAATAATTCTTCACCTTTAGACAT |
| | | | | GACCCTTTTCTCGATATGTT |
| 13 | | P_{BTS1} from | PPGBTS1ps | AAGGGTTGCTCGAGAAAGAGCTC |
| | | SGD | | CCGCCATCTCTACTCACTC |
| 14 | | | PPGBTS1pa | TGAATAATTCTTCACCTTTAGACAT |
| | | | | TGATTTTCCAGACTCGTAAAC |
| 15 | | P_{COG7} from | PPGCOG7ps | AAGGGTTGCTCGAGAAAGAGCTC |
| | | SGD | | CCGGATATGAAAATGGAATGC |
| 16 | | | PPGCOG7pa | TGAATAATTCTTCACCTTTAGACAT |
| | | | | ATTCTGCTTAGTTTGGCCTTC |
| 17 | | P_{GLO2} from | PPGGLO2ps | AAGGGTTGCTCGAGAAAGAGCTC |
| | | SGD | | AGTTCATTGATGTTGAAGAAGTG |
| 18 | | | PPGGLO2pa | TGAATAATTCTTCACCTTTAGACAT |
| | | | | TTTTTGTCCTCCTTTTCTTGTG |
| 19 | P_{RPL25}- | P_{RPL25} (Arm | PGRNRPL25ps | AACGACGGCCAGTGAATTCAGTTTAAACA |
| | KI.LEU2- | 1) from SGD | | TGTACTAATCAGTCTAAC |
| 20 | T_{KI.LEU}-T_{RPL25} | | PGRNRPL25pa | TGGTATATGATTTTGTGGACATTITGCGGC |
| | | | | CGCTTTATCTTATTGATCTTCTTTGTTTAG |
| 21 | | KI.LEU2 from | PGRNKILEU2s | GCGGCCGCAAAATGTCCACAAAATCATAT |
| | | pUG73 | | ACCAG |
| 22 | | | PGRNKILEU2a | TCTAGATTTGGGCCCGATCCCAATACAAC |
| | | | | AGATCA |
| 23 | | T_{RPL25} (Arm | PGRNRPL25ts | CTGTTGTATTGGGATCGGGCCCAAATCTA |
| | | 3) from SGD | | GATCTAATTGGTTTAATTAATAAATTTAATA |
| 24 | | | PGRNRPL25ta | CCTCACGAAGAAGTTAAGCTTGAGCATCG |
| | | | | GACCGAAGCAT |
| 25 | | ARS306 | PGRNARS306s | ATGCTTCGGTCCGATGCTCAAGCTTAACTT |
| | | from SGD | | CTTCGTGAGG |
| 26 | | | PGRNARS306a | GTATGCTATACGAAGTTATTAGGCTCGAG |
| | | | | CTCGAGTTAATTTATCTCATG |
| 27 | P_{YEF3}-RPL25 | P_{YEF3} (2) | PPGRPL25- | GGAATCTCGGTCGTAATGATTT GCATGC |
| | (Arm 2) | from SGD | YEF3ps | ATACATAACATTTTAAGATAAGCAAGTG |
| 28 | | | PPGRPL25- | GCAGTTCACATACCAGATGGAGCCAT |
| | | | YEF3pa | CTTTTAATGTTATCGATGGATTC |
| 29 | | RPL25 | PPGRPL25s | ATGGCTCCATCTGGTATGTGAACTGC |
| | | partial (Arm | | |
| | | 2) from SGD | | |
| 30 | | | PPGRPL25a | GACCATGATTACGCCAAGCTT GTTT |
| | | | | AAACTATGTTCCTTGATACCTC |
| 31 | P_{ERG1}-RPL25 | P_{ERG1} (2) | PPGRPL25- | GGAATCTCGGTCGTAATGATTT GCATGC |
| | (Arm 2) | from SGD | ERG1ps | TGCGATACTGCCGTAGCG |
| 32 | | | PPGRPL25- | GCAGTTCACATACCAGATGGAGCCAT |
| | | | ERG1pa | GACCCTTTTCTCGATATGTT |
| | | RPL25 | PPGRPL25s | As above |
| | | partial (Arm | | |
| | | 2) from SGD | PPGRPL25a | As above |
| 33 | P_{PDA1}-RPL25 | P_{PDA1} (2) | PPGRPL25- | GGAATCTCGGTCGTAATGATTT GCATGC |
| | (Arm 2) | from SGD | PDA1ps | GAAATTCAAAACTCTCCAGAC |
| 34 | | | PPGRPL25- | GCAGTTCACATACCAGATGGAGCCAT |
| | | | PDA1pa | TGGCACAAATGTGGTTTCC |
| | | RPL25 | PPGRPL25s | As above |
| | | partial (Arm | | |
| | | 2) from SGD | PPGRPL25a | As above |
| 35 | P_{BTS1}-RPL25 | P_{BTS1} (2) | PPGRPL25- | GGAATCTCGGTCGTAATGATTT GCATGC |
| | (Arm 2) | from SGD | BTS1ps | CCGCCATCTCTACTCACTC |
| 36 | | | PPGRPL25- | GCAGTTCACATACCAGATGGAGCCAT |
| | | | BTS1pa | TGATTTTCCAGACTCGTAAAC |
| | | RPL25 | PPGRPL25s | As above |
| | | partial (Arm | | |
| | | 2) from SGD | PPGRPL25a | |
| | | | | As above |
| 37 | P_{SEC23}- | P_{SEC23} (2) | PPGSEC23p1s | AACGACGGCCAGTGAATTCAGTTT |
| | hphMX- | from SGD | | AAACTCTTCTGCTTCGTTCAGCTG |
| | T_{SEC23}- | | | |
| | ARSMax1 | | | |
| 38 | | | PPGSEC23p1a | GCACGTCAAGACTGTCAAGGAGGGTATTC |
| | | | | GGGCCCGTATCTTTTTTTCTTTTTTCAAAC |
| 39 | | | | G |
| | | hphMX | PPMLhphs | GACTTAGATTGGTATATATACGCATATG |
| | | pAG32 | | GAATACCCTCCTTGACAGTC |
| 40 | | | PPMLhpha | ATTGATAATGATAAACTCGAACTGACTAGT |
| | | | | CGTTAGTATCGAATCGACAG |
| 41 | | T_{SEC23} (Arm | PPGSEC23ts | GTCGCTATACTGCTGTCGATTCGATACTAA |
| | | 3) from SGD | | CGGCGGCCGCGAGCAACGGCTTTCTTTTG |
| 42 | | | | T |
| | | | | ACAAATGAAAAGAGATGCGGCCGTATGGT |
| | | | PPGSEC23ta | GTGAAAATCT |
| 43 | | ARS1Max | | AGATTTTCACACCATACGGCCGCATCTCTT |
| | | (gBlock) | | TTCATTTGTATTTAAATCCATTTCAAATTTT |
| | | | | ATGTTTAGTTCGAGATCCTCAGTTTTCGGC |
| | | | | GCATAGGAACCACGTACATAATAACTAAA |
| | | | | CATAAATCTATAATAAATAAAAAACAACGA |
| | | | | TGGGAGCTCGAGCCTAATAACTTCGTATA |
| | | | | GCATAC |
| 44 | | | PPGARS1maxa | GTATGCTATACGAAGTTATTAGGCTCGAG |
| | | | | CTCCCATCGTTGTTTTTTATTTATTATAGA |
| 45 | P_{ERG1}-SEC23 | P_{ERG1} (3) | PPGSEC23- | GGAATCTCGGTCGTAATGATTT |
| | (Arm 2) | from SGD | ERG1ps | GATATGAAG GCATGC |
| | | | | TGCGATACTGCCGTAGCG |
| 46 | | | PPGSEC23- | CGTTGATGTCTTCATTAGTCTCGAAGTCCA |
| | | | ERG1pa | T GACCCTTTTCTCGATATGTT |
| 47 | | SEC23 | PPGSEC23s | ATGGACTTCGAGACTAATGAAGACATCAA |
| | | partial (Arm | | CG |
| | | 2) from SGD | | |
| 48 | | | PPGSEC23a | GACCATGATTACGCCAAGCTT GTTTA |
| | | | | AACGTTTCCGTAAGTGATCAAC |
| 49 | P_{GLO2}-SEC23 | P_{GLO2} (2) | PPGSEC23- | GGAATCTCGGTCGTAATGATTT |
| | (Arm 2) | from SGD | GLO2ps | GATATGAAG GCATGC |
| | | | | AGTTCATTGATGTTGAAGAAGTG |
| 50 | | | PPGSEC23- | CGTTGATGTCTTCATTAGTCTCGAAGTCCA |
| | | | GLO2pa | T TTTTTGTCCTCCTTTTCTTGTG |
| | | SEC23 | PPGSEC23s | As above |
| | | partial (Arm | | |
| | | 2) from SGD | | |
| | | | PPGSEC23a | As above |
| 51 | P_{COG7}-SEC23 | P_{COG7} (2) | PPGSEC23- | GGAATCTCGGTCGTAATGATTT |
| | (Arm 2) | from SGD | COG7ps | GATATGAAG GCATGC |
| | | | | CCGGATATGAAAATGGAATGC |
| 52 | | | PPGSEC23- | CGTTGATGTCTTCATTAGTCTCGAAGTCCA |
| | | | COG7pa | T ATTCTGCTTAGTTTGGCCTTC |
| | | SEC23 | PPGSEC23s | As above |
| | | partial (Arm | | |
| | | 2) from SGD | | |
| | | | PPGSEC23a | As above |
| | P_{COG7}-3G- | P_{COG7}-3G (2) | PPGSEC23- | As above |
| | SEC23 (Arm | from SGD | COG7ps | |
| | 2) | | | |
| 53 | SEC23 | | PPGSEC23- | GTTGATGTCTTCATTAGTCTCGAAGTCTCC |
| | partial (Arm | | | TCCTCCCAT |
| | 2) from SGD | | COG7pa1 | ATTCTGCTTAGTTTGGCCTTC |
| | | PPGSEC23s | As above | |
| | | PPGSEC23a | As above | |
| 54 | | P_{RPL33A} from | PPGRPL33As | AAGGGTTGCTCGAGAAAGAGCTC |
| | | SGD | | GTAAAAAGAACAAGAAGAGAATAAAAC |
| 55 | | | PPGRPL33Aa | TGAATAATTCTTCACCTTTAGACAT |
| | | | | TTTTCAATTTATTTGATTGTTGGTTTC |
| 56 | | P_{RPS15} from | PPGRPS15s | AAGGGTTGCTCGAGAAAGAGCTC |
| | | SGD | | CTCGAATAATAACGGCTCTC |
| 57 | | | PPGRPS15a | TGAATAATTCTTCACCTTTAGACAT |
| | | | | GATCGGTCGTGATTATCTTG |
| 58 | | P_{RPC10} from | PPGRPC10s | AAGGGTTGCTCGAGAAAGAGCTC |
| | | SGD | | CCTCGTGTTGTTATAACGAC |
| 59 | | | PPGRPC10a | TGAATAATTCTTCACCTTTAGACAT |
| | | | | TGTTATACTTGTGGACTTTTATTC |
| 60 | | P_{ACT1} from | pACT1s | AAGGGTTGCTCGAGAAAGAGCTCAACCTG |
| | | SGD | | AAGGGACAGAGTTTAAC |
| 61 | | | pACT1a | GTGAATAATTCTTCACCTTTAGACATTGTT |
| | | | | AATTCAGTAAATTTTCGATCTTGGG |
| 62 | | P_{NIP1} from | PPGNIP1s | AAGGGTTGCTCGAGAAAGAGCTC |
| | | SGD | | CGTATCCAATTCGGACGTTG |
| 63 | | | PPGNIP1a | TGAATAATTCTTCACCTTTAGACAT |
| | | | | TTTCGTAGATCTCGGGCTTG |
| 64 | | P_{RPS13} from | PPGRPS13s | AAGGGTTGCTCGAGAAAGAGCTC |
| | | SGD | | ACGTTGAAGAATTGAGGGAG |
| 65 | | | PPGRPS13a | TGAATAATTCTTCACCTTTAGACAT |
| | | | | TTTGACTGATTGTTGTTGATTG |
| 66 | | P_{NUS1} from | PPGNUS1s | AAGGGTTGCTCGAGAAAGAGCTC |
| | | SGD | | AAACGCCACTAATCAACCTG |
| 67 | | | PPGNUS1a | TGAATAATTCTTCACCTTTAGACAT |
| | | | | CTAAGAAAAACAATGGGGAAAATAT |
| 68 | | P_{SMC1} from | PPGSMC1s | AAGGGTTGCTCGAGAAAGAGCTC |
| | | SGD | | AGCTGGAAAAATGCGTAATAAC |
| 69 | | | PPGSMC1a | TGAATAATTCTTCACCTTTAGACAT |
| | | | | TGCGTCTCCTTGTGCCTGCT |
| 70 | | P_{RNA14} from | PPGRNA14s | AAGGGTTGCTCGAGAAAGAGCTC |
| | | SGD | | CAACGTCAACATAATTCAATAG |
| 71 | | | PPGRNA14a | TGAATAATTCTTCACCTTTAGACAT |
| | | | | ATCTCTTGTTTGACTCTCCAG |
| 72 | | P_{RPB7} from | PPGRPB7s | AAGGGTTGCTCGAGAAAGAGCTC |
| | | SGD | | ACCACTGAGGCTAGTGATCT |
| 73 | | | PPGRPB7a | TGAATAATTCTTCACCTTTAGACAT |
| | | | | TCTCAGAAATTGAGTTATTTATAC |
| 74 | | P_{SPC97} from | PPGSPC97s | AAGGGTTGCTCGAGAAAGAGCTC |
| | | SGD | | TTGTGGTGCCACTTTCCGTA |
| 75 | | | PPGSPC97a | TGAATAATTCTTCACCTTTAGACAT |
| | | | | TTTTTCACGCAAGATGTGTAC |
| 76 | | P_{STH1} from | PPGSTH1s | AAGGGTTGCTCGAGAAAGAGCTC |
| | | SGD | | GTTTGATAGCAGTCCATTAAC |
| 77 | | | PPGSTH1a | TGAATAATTCTTCACCTTTAGACAT |
| | | | | TCGCGCTTGCTCTAAACTGTG |
| 78 | | P_{ARP7} from | PPGARP7s | AAGGGTTGCTCGAGAAAGAGCTC |
| | | SGD | | GTAGCGGATGACATCCTGAT |
| 79 | | | PPGARP7a | TGAATAATTCTTCACCTTTAGACAT |
| | | | | TCTTGACAGATCCTTTATAATG |
| 80 | | P_{TAF61} from | PPGTAF61s | AAGGGTTGCTCGAGAAAGAGCTC |
| | | SGD | | GCTTGTTCTCTCGTTGATAC |
| 81 | | | PPGTAF61a | TGAATAATTCTTCACCTTTAGACAT |
| | | | | TGTCGTATTTTATACACACACTG |
| 82 | | P_{RPN11} from | PPGRPN11s | AAGGGTTGCTCGAGAAAGAGCTC |
| | | SGD | | CTGCGGGAACCTCTTCCACA |
| 83 | | | PPGRPN11a | TGAATAATTCTTCACCTTTAGACAT |
| | | | | TATGTCTCGTCTTTCTTGTTAAG |
| 84 | | P_{GAL1}-ERG20- | PIJTERG20s | ACAGGTTCCGGTTAGCCTGC GCTAGC |
| | | P_{RPL3} from | | TTATATTGAATTTTCAAAAATTCTTAC |
| 85 | | pJT9RFR | PIJTERG20a | TTTATTAATTAAACCAATTAGATCTAG |
| | | | | GGGCCC |
| | | | | ATTGTAGCAAAGATTGTAAGGAAATAG |
| 86 | | P_{GAL2}- | PIJTNES1s | CATTACTTCATGAGATAAATTAA |
| | | Y.FAST- | | CTCGAG TGTACTAATCCAAGGAGGTT |
| 87 | | EVBR1.2A- | PIJTNES1a | CTTTGTCTGGAGAGTTTTGAATTTC |
| | | AcNES1- | | GAGCTC ACGCCACAGAAACCTCAGA |
| | | T_{RPL41B} from | | |
| | | pJT9RFR | | |
| 88 | P_{Sk.GAL2}- | P_{Sk.GAL2} from | PSYKSKGAL2ps | GTATCATTACTTCATGAGATAAATTAACTC |
| | Y.FAST- | pILGFP4Q | | GAG TAAACCAATTTTATTTGAACTTGC |
| 89 | EVBR1.2A- | | PSYKSKGAL2pa | CTTACCTTCTTCAATTTTCATTTTGGATCCA |
| | Ec.MBP- | | | CTGTAAAAAACTTTTTTTATTATAC |
| 90 | Linker~SacI~ | Y.FAST- | PTSYFASTs | GTATAATAAAAAAAGTTTTTTACAGTGGAT |
| | 6*G- | EVBR1.2A | | CCAAAATGGAACACGTTGCTTTCG |
| | ERG20^{F96W} | | | |
| | ^{N127W}-T_{RPL3} | from | | |
| | | pJT9RFR | | |
| 91 | | | PITYAFST2Aa | CCAACTTACCTTCTTCAATTTTTGGACCTG |
| | | | | GGTTAAGTTCAAC |
| 92 | | | PITYFAST-MBPS | GCTGGTGACGTTGAACTTAACCCAGGTCC |
| | | | | A AAAATTGAAGAAGGTAAGTTGG |
| 93 | | Ec.MPB | PTSMBPa | ACCACCACCACCACCACCGAGCTCACCAG |
| | | (codon- | | AACCTGGCTTAGTGATTCTAGTTTGGGCA |
| | | optimized) | | TC |
| 94 | | ERG20^{F96W} | PTSERG20s | CCAGGTTCTGGTGAGCTCGGTGGTGGTG |
| | | N^{127W} part 1 | | GTGGTGGTGCTTCAGAAAAAGAAATTAGG |
| | | from pJT11 | | AG |
| 95 | | | Erg20F96Wa | CATATCATCGGCGACCAACCAGTAAGCCT |
| | | | | GCAACAAC |
| 96 | | ERG20^{F96W} | Erg20F96Ws | GTTGTTGCAGGCTTACTGGTTGGTCGCCG |
| | | ^{N127W} part 2 | | ATGATATG |
| | | from pJT11 | | |
| 97 | | | GA_RPL3t_URA3a | AAATCATTACGACCGAGATTCCCGGGATT |
| | | | | GTAGCAAAGATTGTAAGG |
| 98 | | LI.LS from | GA_MBP_LMSS | ATCACTAAGCCAGGTTCTGGTTCTGGTAG |
| | | pJT11 | | AAGATCAGCTAACTATCAACCATCC |
| 99 | | | GA_LMS_6Ga | GAAGCACCACCACCACCACCACCACCCTT |
| | | | | TGTACCTGGTGATGCG |
| 100 | | P_{BTS1}-RPL25 | PMIRPL25BckBns | TTAGCTTATTCTGAGGTTTCTGTGGCGTG |
| | | (Arm2)- | | |
| 101 | | pUC19 from | PMIRPL25BckBna | TCCGGGGTGTTAGACTGATTAGTACATGT |
| | | pILGFP3AA5 | | TT |
| 102 | | P_{ALD6} from | PPGALD6ps | AAGGGTTGCTCGAGAAAGAGCTC |
| | | SGD | | CATATGGCGTATCCAAGCC |
| 103 | | | PPGALD6pa1 | CACAAACACATACTATCAGAATACAGGAT |
| | | | | CCAAAATGTCTAAAGGTGAAGAATTATTCA |
| 104 | | P_{ALD6}- | PILEforReds | CATTACTTCATGAGATAAATTAA CTCGAG |
| | | AeBlue-T_{PGK1} | | CATATGGCGTATCCAAGCC |
| 105 | | (_{PALD6}- | PILEforReda | AAATCATTACGACCGAGATTCCCGGG |
| | | EforRed- | | AAATAATATCCTTCTCGAAAGC |
| | | T_{PGK1}) | | |
| 106 | P_{Se.GAL2}- | P_{Se.GAL2} from | PHPVSeGAL2ps | GCTTTCGAGAAGGATATTATTTCCCGGGC |
| | HPV16L1AC1 | pILGFP4M | | CACAGAGAACAGGAGATTAC |
| | 4-6*H- | | | |
| | T_{RPL41B} | | | |
| 107 | | | PHPVSeGAL2pa | AGATGGCAACCACAAAGACATTTTGTCGA |
| | | | | CTGTAAATGTGTGTATATATTATATTATAG |
| 108 | | HPV16L1AC1 | PHPVHPV16Ls | CTATAATATAATATATACACACATTTACAG |
| | | 4-6*H | | TCGACAAAATGTCTTTGTGGTTGCCATCT |
| | | (codon | | |
| | | optimized) | | |
| | | from gBlock | | |
| 109 | | | PHPVHPV16La | TCCGCCCTGCAGGTCACTATTAATGATGG |
| | | | | TGATGGTGGTGAGCAGTTGTAGAGGTAGA |
| | | | | AG |
| 110 | | T_{RPL41B} from | PHPVRPL41Bts | ACTGCTCACCACCATCACCATCATTAATAG |
| | | SGD | | TGACCTGCAGGGCGGATTGAGAGCAAATC |
| | | | | G |
| 111 | | | PHPVRPL41Bta | GCATGCAAATCATTACGACCGAGATTGCC |
| | | | | GGCACGCCACAGAAACCTCAGAAT |
| 112 | | P_{ALD6}- | PHPVALD6ps | GGGCGAATTGGGTACCGGGCCC |
| | | AeBlue- | | CATATGGCGTATCCAAGCCG |
| | | T_{PGK1}- | | |
| | | PSe._{GAL2}- | | |
| | | HPV16L1ΔC1 | | |
| | | 4-6*H- | | |
| | | T_{RPL41B} | | |
| 113 | | | PHPVRPL41Bta | CACTAAAGGGAACAAAAGCTGGAGCTC |
| | | | | CGCCACAGAAACCTCAGAAT |
| | | HPV16L1ΔC2 | PHPVHPV16Ls | As above |
| | | 2-6*H | | |
| 114 | | | PHPVHPV16aad | GCCCTGCAGGTCACTATTAATGATGGTGA |
| | | | a | TGGTGGTGACCCAAAGTGAACTTTGGCTT |
| | | | | AG |
| 115 | | | PHPVHPV16a | GATTTGCTCTCAATCCGCCCTGCAGGTCA |
| | | | | CTATTA |
| 116 | Removing | | PMIRPL25ta | CCTCACGAAGAAGTTAAGCTTGAGCATCG |
| | ARS in | | | GACCGAAGCATAAG |
| | Construct 3 | | | |
| 117 | | | PMITEF1s | ATTACTTCATGAGATAAATTAACCTGCAGG |
| | | | | CGTATAAACAATGCATACTTTGTAC |

TABLE 5
Construction of the plasmids used in this work. Numbers refer to DNA fragments listed in Table 4.

| Plasmid | Construction process |
|---|---|
| pILGFP1D5 | Fragment T_{PGK1} (#1) was cloned into SpeI of pILGFP3 through Gibson |
| | Assembly to generate plasmid pILGFP1D5 |
| pILGFP5A3 | Fragment P_{YEF3} (#2) was cloned into BamHI site of plasmid |
| | pILGFP1D5 through Gibson Assembly to generate plasmid |
| | pILGFP5A3, and: |
| pILGFP1A6 | Fragment P_{RPL25} (#3) to generate plasmid pILGFP1A6 |
| pILGFP1C6 | Fragment P_{SEC23} (#4) to generate plasmid pILGFP1C6 |
| pILGFP1E6 | Fragment P_{PDA1} (#5) to generate plasmid pILGFP1E6 |
| pILGFP1E7 | Fragment P_{ERG1} (#6) to generate plasmid pILGFP1E7 |
| pILGFP1G7 | Fragment P_{BTS1} (#7) to generate plasmid pILGFP1G7 |
| pILGFP4F5 | Fragment P_{COG7} (#8) to generate plasmid pILGFP4F5 |
| pILGFP4H5 | Fragment P_{GLO2} (#9) to generate plasmid pILGFP4H5 |
| pILGFP6G3 | Fragment P_{RPL33A} (#20) to generate plasmid pILGFP6G3 |
| pILGFP6A4 | Fragment P_{RPS15} (#21) to generate plasmid pILGFP6A4 |
| pILGFP6C4 | Fragment P_{RPC10} (#22) to generate plasmid pILGFP6C4 |
| pACT1-GFP | Fragment P_{ACT1} (#23) to generate plasmid pACT1-GFP |
| pILGFP6G4 | Fragment P_{NIP1} (#24) to generate plasmid pILGFP6G4 |
| pILGFP6A5 | Fragment P_{RPS13} (#25) to generate plasmid pILGFP6A5 |
| pILGFP6C5 | Fragment P_{NUS1} (#26) to generate plasmid pILGFP6C5 |
| pILGFP6E5 | Fragment P_{SMC1} (#27) to generate plasmid pILGFP6E5 |
| pILGFP6G5 | Fragment P_{RNA14} (#28) to generate plasmid pILGFP6G5 |
| pILGFP6A6 | Fragment P_{RPB7} (#29) to generate plasmid pILGFP6A6 |
| pILGFP6C6 | Fragment P_{SPC97} (#30) to generate plasmid pILGFP6C6 |
| pILGFP6E6 | Fragment P_{STH1} (#31) to generate plasmid pILGFP6E6 |
| pILGFP6G6 | Fragment P_{ARP7} (#32) to generate plasmid pILGFP6G6 |
| pILGFP6A7 | Fragment P_{TAF61} (#33) to generate plasmid pILGFP6A7 |
| pILGFP6C7 | Fragment P_{RPN11} (#34) to generate plasmid pILGFP6C7 |
| pILGFP1DFB | Fragment P_{RPL25}-KI.LEU2-T_{KI.LEU}-T_{RPL25} (#10) was cloned into EcoRI/XbaI |
| | sites of pILGFP89 through Gibson assembly to generate plasmid |
| | pILGFP1DFB |
| pILGFP3A5C | Fragment P_{YEF3}-RPL25 (Arm 2) (#11) was cloned into SphI site of |
| | plasmid pILGFP1DFB through Gibson assembly to generate |
| | plasmid pILGFP3A5C, and: |
| pILGFP3AE4 | Fragment P_{ERG1}-RPL25 (Arm 2) (#12) to generate pILGFP3AE4 |
| pILGFP3AG4 | Fragment P_{PDA1}-RPL25 (Arm 2) (#13) to generate pILGFP3AG4 |
| pILGFP3AA5 | Fragment P_{PST1}-RPL25 (Arm 2) (#14) to generate pILGFP3AA5 |
| pILGFP3AG4ARSd | pILGFP3AG4 was used as the template to amplify fragment #46, which |
| | was self-ligated to generate plasmid pILGFP3AG4ARSd. |
| pILGFP4BG6 | Fragment P_{SEC23}-hphMX-T_{SEC23}-ARSMax1 (#15) was cloned into EcoRI/XbaI |
| | sites of pILGFP89 through Gibson assembly to generate plasmid |
| | pILGFP4BG6 |
| pILGFP5EG3 | Fragment P_{ERG1}-SEC23 (Arm 2) (#16) was cloned into SphI site of |
| | plasmid pILGFP4BG6 through Gibson assembly to generate |
| | plasmid pILGFP5EG3, and: |
| pILGFP5EA4 | Fragment P_{GLO2}-SEC23 (Arm 2) (#17) to generate plasmid pILGFP5EA4 |
| pILGFP5EC4 | Fragment P_{COG7}-SEC23 (Arm 2) (#18) to generate plasmid pILGFP5EC4 |
| pILGFP5EF3 | Fragment P_{COG7}-3G-SEC23 (Arm 2) (#19) to generate plasmid pILGFP5EC4 |
| pINER2R | Step 1: Fragment P_{GAL1}-ERG20-P_{RPL3} (#35) was cloned into ApaI site of |
| | plasmid pILGFP3AE4 through Gibson assembly to generate plasmid |
| | pITinter1. |
| | Step 3: Fragment P_{GAL2}-Y.FAST-EVBR1.2A-AcNES1-T_{RPL41B} (#36) was |
| | cloned into SacI/Xmal sites of plasmid pITinter1 through Gibson assembly |
| | to generate pINER2R |
| pINER3R | Step 1: Fragment P_{GAL1}-ERG20-P_{RPL3} (#35) was cloned into ApaI site of |
| | plasmid pILGFP3AG4 through Gibson assembly to generate plasmid |
| | pITinter2. |
| | Step 3: Fragment P_{GAL2}-Y.FAST-EVBR1.2A-AcNES1-T_{RPL41B} (#36) was |
| | cloned into SacI/XmaI sites of plasmid pITinter2 through Gibson assembly |
| | to generate pINER3R |
| pINER4R | Step 1: Fragment P_{GAL1}-ERG20-P_{RPL3} (#35) was cloned into ApaI site of |
| | plasmid pILGFP3AA5 through Gibson assembly to generate plasmid |
| | pITinter3. |
| | Step 3: Fragment P_{GAL2}-Y.FAST-EVBR1.2A-AcNES1-T_{RPL41B} (#36) was |
| | cloned into SacI/XmaI sites of plasmid pITinter3 through Gibson assembly |
| | to generate pINER3R |
| pIT6EG7m | Fragment P_{Sk.GAL2}-Y.FAST-EVBR1.2A-Ec.MBP-Linker~SacI~6*G-ERG20^{F96W N127W}- |
| | T_{RPL3} (#37) was cloned into XhoI/XmaI sites of pILGFP3AG4 to |
| | generate pIL6EG7m |
| pIT6EG7ml | Fragment LI.LS (#38) was cloned into XhoI/XmaI sites of pILGFP3AG4 |
| | through Gibson assembly to generate pIL6EG7ml |
| pIT6EG7mlh | Fragment P_{BTS1}-RPL25 (Arm2)-pUC19 (#39) was assembled with the larger |
| | fragment of PmeI/SmaI-digested plasmid pIT6EG7ml to generate plasmid |
| | pIT6EG7mlh |
| pPT6EG7ml | P_{Sk.GAL2} > Y.FAST-EVBR1.2A-Ec.MBP-Linker~SacI~6*G-ERG20^{F36W N127W} > |
| | T_{RPL3} was cut out from pIT6EG7ml with XhoI and XmaI and cloned |
| | into XhoI/XmaI sites in pRS425 to generate pPT6EG7ml. |
| pILAC2 (or pILAC3) | Step 1: plasmid pLAC1 was digested with NotI, and then mung bean |
| | nuclease; and further purified through a PCR clean-up kit. |
| | Step 2: Step 1 product was digested with EcoRI and XmaI, and the larger |
| | fragment was purified through a Gel-cutting purification kit. |
| | Step 3: plasmid pILGFP3AG4 (or pILGFP3AA5) was digested with XhoI, |
| | plasmid pLacI was digested with NotI, and then mung bean nuclease; and |
| | further purified through a PCR clean-up kit. |
| | Step 4: Step 3 product was digested with XmaI, and the larger fragment |
| | was purified through a Gel-cutting purification kit. |
| | Step 5: Step 2 product and Step 4 product were ligated to generate |
| | pILAC2 (or pILAC3). |
| pIAeBlue (or | Step 1: Fragment P_{ALD6} (#40) was cloned into BamHI site of plasmid |
| pIEforRed) | pILGFP1D5 through Gibson Assembly to generate plasmid pILGFP4D2. |
| | Step 2: gBlock fragment AeBlue (or EforRed) with codon usage optimized |
| | was cloned into BamHI/Bg/II sites of plasmid pILGFP4D2 through Gibson |
| | Assembly to generate plasmid pILAeBlue (or pILEforRed) |
| | Step 3: Fragment P_{ALD6}-AeBlue-T_{PGK1} (#41) (or P_{ALD6}-EforRed-T_{PGK1;} #42) |
| | was amplified from pILAeBlue (or pILEforRed) and cloned into XhoI/XmaI |
| | sites of pILGFP3AA5 through Gibson assembly to generate pIAeBlue (or |
| | pIEforRed). |
| pIAeBlueHPV16LR | Step 1: Fragment P_{Se.GAL2}-HPV16L1ΔC14-6*H-T_{RPL41B} (#43) was cloned into |
| | SmaI site of plasmid pIAeBlue to generate pIAeBlueHPV16L. |
| | Step 2: Fragment HPV16L1ΔC22-6*H (#45) was cloned Sa/I/SbfI sites of |
| | pIAeBlueHPV16L to generate pIAeBlueHPV16LR. |
| pPAeBlueHPV16LR | Step 1: Fragment P_{ALD6}-AeBlue-TPGK1-PSe_{.GAL2}-HPV16L1ΔC14-6*H-T_{RPL41B} |
| | (#44) amplified from pIAeBlueHPV16L was cloned into ApaI/SacI sites of |
| | plasmid pRS425 to generate pPAeBlueHPV16L. |
| | Step 2: Fragment HPV16L1ΔC22-6*H (#45) was cloned Sa/I/SbfI sites of |
| | pPAeBlueHPV16L to generate pPAeBlueHPV16LR. |

TABLE 6
Construction of the ILHA series strains used in this work. Plasmids
refer to Table S1. DNA fragments refer to Table S3.

| Strain | Construction process |
|---|---|
| G5A3 | Plasmid pILGFP5A3 digested with SwaI was transformed into |
| | CEN.PK113-5D to generate strain G5A3, and: |
| G1A6 | pILGFP1A6 to generate strain G1A6 |
| G1C6 | pILGFP1C6 to generate strain G1C6 |
| G1E6 | pILGFP1E6 to generate strain G1E6 |
| G1E7 | pILGFP1E7 to generate strain G1E7 |
| G1G7 | pILGFP1G7 to generate strain G1G7 |
| G4F5 | pILGFP4F5 to generate strain G4F5 |
| G4H5 | pILGFP4H5 to generate strain G4H5 |
| G6G3 | pILGFP6G3 to generate strain G6G3 |
| G6A4 | pILGFP6A4 to generate strain G6A4 |
| G6C4 | pILGFP6C4 to generate strain G6C4 |
| G6E4 | pILGFP6E4 to generate strain ACT1-GFP |
| G6G4 | pILGFP6G4 to generate strain G6G4 |
| G6A5 | pILGFP6A5 to generate strain G6A5 |
| G6C5 | pILGFP6C5 to generate strain G6C5 |
| G6E5 | pILGFP6E5 to generate strain G6E5 |
| G6G5 | pILGFP6G5 to generate strain G6G5 |
| G6A6 | pILGFP6A6 to generate strain G6A6 |
| G6C6 | pILGFP6C6 to generate strain G6C6 |
| G6E6 | pILGFP6E6 to generate strain G6E6 |
| G6G6 | pILGFP6G6 to generate strain G6G6 |
| G6A7 | pILGFP6A7 to generate strain G6A7 |
| G6C7 | pILGFP6C7 to generate strain G6C7 |
| G3A5C | pILGFP3A5C to generate strain G3A5C |
| G3AE4 | pILGFP3AE4 to generate strain G3AE4 |
| G3AG4 | pILGFP3AG4 to generate strain G3AG4 |
| G3AA5 | pILGFP3AA5 to generate strain G3AA5 |
| G5EG3 | pILGFP5EG3 to generate strain G5EG3 |
| G5EA4 | pILGFP5EA4 to generate strain G5EA4 |
| G5EC4 | pILGFP5EC4 to generate strain G5EC4 |
| G5EF3 | pILGFP5EF3 to generate strain G5EF3 |
| o401UR | Plasmid pIR3DH8 digested by PmeI was transformed into strain o401R to |
| | generate strain o401UR |
| N401-1 | Plasmid pJT9RFR was transformed into strain o401UR to generate strain |
| | N401-1 |
| N401-2 | Plasmid pINER2R digested by PmeI was transformed into strain o401UR to |
| | generate strain N401-2 |
| N401-3 | Plasmid pINER3R digested by PmeI was transformed into strain o401UR to |
| | generate strain N401-3 |
| N401-4 | Plasmid pINER4R digested by PmeI was transformed into strain o401UR to |
| | generate strain N401-4 |
| LIM141R/ | o141R derivative; |
| LIM141R2 | [pPT6EG7ml] |
| LIM141M | Plasmid pIT6EG7ml digested by PmeI was transformed intro strain o141R to |
| | generate strain N141M |
| LIM141MH | Plasmid pIT6EG7mlh digested by PmeI was transformed intro strain o141R to |
| | generate strain N141MH |
| LAC4 | Plasmid pILAC2 digested by PmeI was transformed into strain o401UR to |
| | generate strain LAC4 |
| LAC5 | Plasmid pILAC3 digested by PmeI was transformed into strain o401UR to |
| | generate strain LAC5 |
| 16BJ3 | Plasmid pIR3DH8 digested by PmeI was transformed into strain CEN.PK113- |
| | 16B to generate strain 16BJ3 |
| 16BJ3C | Plasmid pRS425 was transformed into strain 16BJ3 to generate strain 16BJ3C |
| 16BJ3AeBlue | Plasmid pIAeBlue digested by PmeI was transformed into strain 16BJ3 to |
| | generate strain 16BJ3AeBlue |
| HPV16LPR | Plasmid pPAeBlueHPV16L1R was transformed into strain 16BJ3 to generate |
| | strain HPV16LPR |
| HPV16LMR | Plasmid pIAeBlueHPV16L1R digested by PmeI was transformed into strain |
| | 16BJ3 to generate strain HPV16LPR |

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1-25. (canceled)

26. A method for increasing copy number of a nucleic acid construct in the genome of a yeast cell, wherein the nucleic acid construct comprises a heterologous nucleic acid sequence and a recombinant polynucleotide, the method comprising:
introducing the nucleic acid construct into the genome, wherein the heterologous nucleic acid sequence is introduced in operable connection with a endogenous haploinsufficient gene of the genome; and
reducing expression of the endogenous haploinsufficient gene, wherein the recombinant polynucleotide reduces expression of the endogenous haploinsufficient gene and the reduced expression of the endogenous haploinsufficient gene increases copy number in the genome of the nucleic acid construct and the endogenous haploinsufficient gene, thereby increasing the copy number of the heterologous nucleic acid sequence in the genome of the cell.

27. The method of claim 26, wherein the heterologous nucleic sequence comprises at least one coding sequence in operable connection with a promoter that is operable in the cell.

28. The method of claim 26, wherein the nucleic acid construct comprises an origin of replication.

29. The method of claim 26, wherein the recombinant polynucleotide of the nucleic acid construct is selected from the group consisting of:
(a) a polynucleotide that comprises a promoter that is weaker than the promoter of the endogenous haploinsufficient gene, which when introduced into the genome of the cell, is operably connected to the endogenous haploinsufficient gene;
(b) a modified haploinsufficient gene that is distinguished from the endogenous haploinsufficient gene by replacement of the endogenous promoter of the endogenous haploinsufficient gene with a weaker promoter;
(c) a modified haploinsufficient gene that is distinguished from the endogenous haploinsufficient gene by replacement of at least one codon of the haploinsufficient gene with a codon that has a lower translational efficiency in the cell than the codon it replaces:
(d) a modified haploinsufficient gene that is distinguished from the endogenous haploinsufficient gene by disruption of endogenous haploinsufficient gene;
(e) a modified haploinsufficient gene that is distinguished from the endogenous haploinsufficient gene by operably connecting a nucleotide sequence encoding an RNA destabilizing element to the endogenous haploinsufficient gene; and
(f) a polynucleotide that reduces the level of an expression product of the haploinsufficient gene.

30. The method of claim 29, wherein the recombinant polynucleotide of the nucleic acid construct is a polynucleotide that comprises a promoter that is weaker than the promoter of the endogenous haploinsufficient gene, which when introduced into the genome of the cell, is operably connected to the endogenous haploinsufficient gene, or a modified haploinsufficient gene that is distinguished from the endogenous haploinsufficient gene by replacement of the endogenous promoter of the endogenous haploinsufficient gene with a weaker promoter.

31. The method of claim 26, wherein the increased copy number of the endogenous haploinsufficient gene or the nucleic acid construct is from 2 to 200 copies, suitably 3 to 100 copies, suitably 3 to 70 copies, suitably 3 to 60 copies.

32. The method of claim 26, wherein the endogenous haploinsufficient gene is selected from the group consisting of RPL25, SEC23, RPL33A, RPS15, RPC10, RPS5, ACT1, NIP1, RPS13, NUS1, SMC1, RNA14, RPB7, SPC97, STH1, ARP7, TAF61 and RPN11.

33. The method of claim 30, wherein the weaker promoter is selected from the group consisting of ERG1 promoter, PDA1 promoter, BTS1 promoter, GLO2 promoter and COG7 promoter.

34. The method of claim 32, wherein the endogenous haploinsufficient gene is operably connected to an origin of replication, wherein the origin of replication is ARS306 or ARS1max.

35. A genetically modified yeast cell, comprising a nucleic acid construct in its genome, wherein the nucleic acid construct comprises: (1) a recombinant polynucleotide that reduces expression of a haploinsufficient gene that is endogenous to the cell of interest; and (2) a heterologous nucleic acid sequence in operable connection with the haploinsufficient gene, wherein the heterologous nucleic sequence comprises at least one coding sequence in operable connection with a promoter that is operable in the cell.

36. The genetically modified yeast cell of claim 35, wherein the nucleic acid construct further comprises an origin of replication.

37. The genetically modified yeast cell of claim 36, wherein the recombinant polynucleotide of the nucleic acid construct is selected from the group consisting of:
(a) a polynucleotide that comprises a promoter that is weaker than the promoter of the endogenous haploinsufficient gene, which when introduced into the genome of the cell, is operably connected to the endogenous haploinsufficient gene;
(b) a modified haploinsufficient gene that is distinguished from the endogenous haploinsufficient gene by replacement of the endogenous promoter of the endogenous haploinsufficient gene with a weaker promoter;
(c) a modified haploinsufficient gene that is distinguished from the endogenous haploinsufficient gene by replacement of at least one codon of the haploinsufficient gene with a codon that has a lower translational efficiency in the cell than the codon it replaces:
(d) a modified haploinsufficient gene that is distinguished from the endogenous haploinsufficient gene by disruption of endogenous haploinsufficient gene;
(e) a modified haploinsufficient gene that is distinguished from the endogenous haploinsufficient gene by operably connecting a nucleotide sequence encoding an RNA destabilizing element to the endogenous haploinsufficient gene; and
(f) a polynucleotide that reduces the level of an expression product of the haploinsufficient gene.

38. The genetically modified yeast cell of claim 37, wherein:
the haploinsufficient gene is ribosomal 60S subunit protein L25 or GTPase-activating protein SEC23;
the weaker promoter is selected from the group consisting of ERG1 promoter, PDA1 promoter, BTS1 promoter, GLO2 promoter and COG7 promoter; and
the origin of replication is the autonomous replicating sequence ARS306 or ARS1max.

39. A nucleic acid construct comprising a recombinant polynucleotide that reduces expression of a haploinsufficient gene that is endogenous to a yeast cell of interest.
